(19) United States
(12) Patent Application Publication
(10) Pub. No.: US 2025/0189496 A1
(43) Pub. Date: Jun. 12, 2025

(54) PREDICTING CHEMICAL STRUCTURE AND PROPERTIES BASED ON MASS SPECTRA

(71) Applicant: Enveda Therapeutics, Inc., Boulder, CO (US)

(72) Inventors: David Wendell HEALEY, Boulder, CO (US); Thomas Charles BUTLER, Boulder, CO (US); Joseph Douglas DAVISON, Boulder, CO (US); Nicholas Rex BOYCE, Boulder, CO (US); Brian Hamilton BARGH, Boulder, CO (US); Gennady VORONOV, Boulder, CO (US)

(21) Appl. No.: 18/840,882

(22) PCT Filed: Feb. 22, 2023
(86) PCT No.: PCT/US2023/063082

§ 371 Date: Aug. 22, 2024

Related U.S. Application Data
(60) Provisional application No. 63/410,529, filed on Sep. 27, 2022.
(60) Provisional application No. 63/351,688, filed on Jun. 13, 2022.
(60) Provisional application No. 63/313,223, filed on Feb. 23, 2022.

(51) Int. Cl. G01N 30/72; G01N 30/02; G01N 30/86; H01J 49/00

(52) U.S. Cl. CPC G01N 30/7266; G01N 30/8693; H01J 49/004; G01N 2030/027

(57) ABSTRACT

Methods for identifying a chemical structure of a compound based on mass spectrometry (MS) data using one or more computing devices are disclosed. The methods include receiving mass spectrometry (MS) data that includes a plurality of mass-to-charge values associated with fragments obtained from mass spectrometry performed on the compound, inputting the plurality of mass-to-charge values into a tokenizer trained to generate a plurality of tokens based on the plurality of mass-to-charge values, and determining one or more chemical structures of the compound based at least in part on the plurality of tokens.

### CROSS-REFERENCE TO RELATED APPLICATIONS

This application claims priority benefit of U.S. Provisional Patent Application No. 63/313,223, filed 23 Feb. 2022; U.S. Provisional Patent Application No. 63/351,688 filed 13 Jun. 2022; and U.S. Provisional Patent Application No. 63/410,529 filed 27 Sep. 2022. The entire contents of each of those patent applications are hereby incorporated by reference herein.

### TECHNICAL FIELD

This application relates generally to mass spectra, and, more particularly, to predicting chemical structures and chemical properties based on mass spectra including precursor mass.

### BACKGROUND

Mass spectrometry (MS) is a well-used technique for studying molecules present in a sample, including samples originating from individuals or plants. Certain MS techniques include fragmenting a parent molecule (such as a small chemical compound) in the sample, for example, using collision-induced dissociation (CID) or electron-transfer dissociation (ETD), followed by measuring mass-to-charge (m/z) value of the resulting ionized fragments of the parent molecule to generate a tandem mass spectrum. Determining the chemical structure of the molecule from the sample using only a mass spectrum is often impracticable due, at least in part, to the high degree of chemical structure diversity that exists in nature. This impracticality only increases when trying to broadly characterize complex samples that may contain hundreds, if not thousands, of different molecules. Moreover, mass spectrometry data is inherently noisy, for example, due to the presence of volatile compounds or electric noise, and this noise may confound confident identification of a molecule. It may thus be useful to provide techniques for identifying molecules from acquired mass spectra.

### SUMMARY

Embodiments of the present embodiments are directed toward a computational metabolomics platform that may be utilized to predict the chemical structure of a molecule, compound, or small molecule (e.g., metabolite) based on the known mass spectra and precursor mass to identify a molecule, a compound, or a small molecule (e.g., metabolite) that may have been previously scientifically unidentified. For example, in certain embodiments, the computational metabolomics platform, utilizing one or more trained bidirectional transformer-based machine-learning models (e.g., a bidirectional and auto-regressive transformer (BART) model, a bidirectional encoder representations for transformer (BERT) model, a generative pre-trained transformer (GPT) model, or some combination of a BERT model and a GPT model), may predict and generate the chemical structure and/or chemical properties of a molecule, compound, or small molecule (e.g., metabolites) based on only the known mass spectrometry (MS) data, which may include mass-to-charge (m/z) values and precursor mass (e.g., precursor m/z) value.

Indeed, the computational metabolomics platform, utilizing one or more trained bidirectional transformer-based machine-learning models (e.g., a BART model, a BERT model, a GPT model), may predict, generate, and store the chemical structure (e.g., 2D chemical structure, 3D chemical conformation, and so forth) and chemical properties for various naturally-occurring and/or non-naturally-occurring molecules, compounds, or small molecules (e.g., metabolites) that-without the presently disclosed embodiments-would otherwise remain scientifically unidentified. In this way, the present embodiments may allow for increased inferences that may be drawn from such molecules, compounds, or small molecules (e.g., metabolites) at scale without having necessarily to isolate each molecule or compound included within a given naturally-occurring chemical or biochemical sample. Such techniques may further facilitate and expedite the drug discovery process with respect to various small molecule medicines, small molecule therapeutics, small molecule vaccines, small molecule antibodies, small molecule antivirals, and so forth.

### BRIEF DESCRIPTION OF THE DRAWINGS

FIG. 1A illustrates an example embodiment of a workflow diagram of an inference phase of a bidirectional transformer-based machine-learning model trained to generate predictions of the chemical structure of a compound based on MS data.

FIG. 1B illustrates a flow diagram of a method for generating predictions of the chemical structure of a compound based on tokenizations of MS data.

FIG. 1C illustrates a flow diagram of a method for generating predictions of the chemical structure of a compound based on tokenizations of MS data, including precursor mass.

FIG. 2A illustrates an example embodiment of a workflow diagram of a pre-training phase for a bidirectional transformer-based machine-learning model to generate predictions of the chemical structure of a compound utilizing SMILES strings.

FIG. 2B illustrates a flow diagram of a method for pre-training and fine-tuning a bidirectional transformer-based machine-learning model to generate predictions of the chemical structure of a compound utilizing SMILES strings.

FIG. 2C illustrates an example embodiment of a workflow diagram of a training phase for pre-training and fine-tuning a bidirectional transformer-based machine-learning model for to generate predictions of the chemical structure of a compound utilizing MS data.

FIG. 2D illustrates a flow diagram of a method for pre-training and fine-tuning a bidirectional transformer-based machine-learning model to generate predictions of the chemical structure of a compound utilizing MS data.

FIGS. 2E-2G illustrate one or more running examples for pre-training, fine-tuning, and inference for a bidirectional transformer-based machine-learning model to generate predictions of the chemical structure of a compound.

FIG. 2H illustrates a flow diagram of a method for utilizing a bidirectional transformer-based machine-learning model pre-trained and fine-tuned to generate predictions of the chemical structure of a compound based on sinusoidal embeddings of MS data.

FIG. 2I illustrates a running example of the inference phase of a bidirectional transformer-based machine-learning model pre-trained and fine-tuned to generate predictions of the chemical structure of a compound based on sinusoidal embeddings of MS data.

FIG. 2J illustrates a flow diagram of a method for pre-training and fine-tuning a bidirectional transformer-based machine-learning model to generate predictions of the chemical structure of a compound utilizing MS data, including precursor mass.

FIG. 2K illustrates one or more running examples for pre-training, fine-tuning, and inference for a bidirectional transformer-based machine-learning model to generate predictions of the chemical structure of a compound utilizing MS data, including precursor mass.

FIG. 3A illustrates a flow diagram of a method for providing a subword tokenizer to be utilized with a bidirectional transformer-based machine-learning model to generate predictions of the chemical structure of a compound.

FIG. 3B illustrates a flow diagram of a method for training a subword tokenizer to be utilized with a bidirectional transformer-based machine-learning model to generate predictions of the chemical structure of a compound.

FIG. 3C illustrates an example embodiment of a workflow diagram for training a subword tokenizer to be utilized with a bidirectional transformer-based machine-learning model to generate predictions of the chemical structure of a compound.

FIG. 4A illustrates a flow diagram of a method for generating predictions of one or more chemical properties of a compound based on MS data.

FIG. 4B illustrates a running examples for generating predictions of one or more chemical properties of a compound based on MS data.

FIG. 4C illustrates a flow diagram of a method for generating predictions of one or more chemical properties of a compound based on MS data including precursor mass.

FIG. 5A illustrates a flow diagram of a method for generating training data for a bidirectional transformer-based machine-learning model trained to generate predictions of the chemical structure of a compound based on MS data.

FIG. 5B illustrates a running examples for generating training data for a bidirectional transformer-based machine-learning model trained to generate predictions of the chemical structure of a compound based on MS data.

FIG. 6 illustrates an example computing system included as part of an exemplary computational metabolomics platform.

FIG. 7 illustrates a diagram of an example artificial intelligence (AI) architecture included as part of an exemplary computational metabolomics platform.

### DETAILED DESCRIPTION

Described are systems and methods for predicting the chemical structure of a molecule, compound, or small molecule (e.g., metabolite) based on only the known mass spectra and precursor mass to identify a molecule, a compound, or a small molecule (e.g., metabolite) that may have been previously scientifically unidentified. For example, in certain embodiments, the computational metabolomics platform, utilizing one or more trained bidirectional transformer-based machine-learning models (e.g., a BART model, a BERT model, a GPT model), may predict and generate the chemical structure and/or chemical properties of a molecule, compound, or small molecule (e.g., metabolites) based on only the known mass spectrometry (MS) data, which may include mass-to-charge (m/z) values and precursor mass (e.g., precursor m/z) value.

The computational metabolomics platform, utilizing one or more trained bidirectional transformer-based machine-learning models (e.g., a BART model, a BERT model, a GPT model), may predict, generate, and store the chemical structure (e.g., 2D chemical structure, 3D chemical conformation, and so forth) and chemical properties for various naturally-occurring and/or non-naturally-occurring molecules, compounds, or small molecules (e.g., metabolites) that-without the presently disclosed embodiments-would otherwise remain scientifically unidentified. In this way, the present embodiments may allow for increased inferences that may be drawn from such molecules, compounds, or small molecules (e.g., metabolites) at scale without having necessarily to isolate each molecule or compound included within a given naturally-occurring chemical or biochemical sample. Such techniques may further facilitate and expedite the drug discovery process with respect to various small molecule medicines, small molecule therapeutics, small molecule vaccines, small molecule antibodies, small molecule antivirals, and so forth.

### 1. Mass Spectrometry (MS) and MS Data Overview

Analysis of a compound or compounds using mass spectrometry is known in the art. Numerous types of information and data points can be obtained from such mass spectrometry analyses, one or more of which may be included as MS data described herein.

In some embodiments, the MS data comprises a parent molecule (parent ion) mass-to-charge (m/z) value. In some embodiments of the description provided herein, the parent molecule is referred to as the precursor molecule, and includes extensions of the term such as precursor m/z and precursor mass. In some embodiments, the parent molecule m/z value is converted to a mass, such as determined based on a parent molecule m/z value and the charge of the parent ion. In some embodiments, the MS data comprise a parent molecule abundance (relative intensity). In some embodiments, the MS data comprises a parent molecule attribute based on the LC-MS or MS techniques used to acquire data on the parent molecule, such as LC retention time, positive or negative charge (positive or negative mode), and m/z value window used during data acquisition.

In some embodiments, the MS data comprises a plurality of mass-to-charge (m/z) values associated with fragments of a parent molecule obtained from mass spectrometry performed on a compound, such as tandem mass spectrometry. In some embodiments, the fragment molecule m/z value is converted to a mass, such as determined based on a fragment molecule m/z value and the charge of the fragment ion. In some embodiments, the plurality of m/z values are derived from a mass spectrum. In some embodiments, the plurality of m/z values are derived from mass spectra, such as acquired in one or more mass spectrometry analyses. In some embodiments, the plurality of m/z values represent a sub-population of m/z values obtained from one or more mass spectra, such as based on an attribute of the mass spectrometry technique or acquired data, e.g., such as intensity or relative abundance of m/z values (e.g., highest intensity m/z values or those above a certain intensity or relative abundance threshold). In some embodiments, MS data comprises a plurality of mass values based on m/z values obtained from a mass spectrometry. In some embodiments, mass values may assume or predict a charge value associate with a compound and/or fragment thereof (e.g., a single m/z value converted to a number of mass values within a range of possible charges of the compound and/or fragment thereof).

In some embodiments, the MS data comprises a plurality of mass-to-charge (m/z) values associated with fragments of a parent molecule, and the associated parent molecule m/z value and/or mass. In some embodiments, the MS data comprises a plurality of mass-to-charge (m/z) values associated with fragments of a parent molecule, and does not include the associated parent molecule m/z value and/or mass.

In some embodiments, the MS data comprises intensity or relative abundance information associated with an m/z value. In some embodiments, the intensity or relative abundance information is an averaged and/or normalized intensity or relative abundance value, e.g., averaged according to mass spectra and/or normalized relative to a reference or standard.

In some embodiments, the MS data comprises ion mobility data derived from an ion mobility mass spectrometry technique. In some embodiments, the MS data comprises a collisional cross section of a compound or a fragment thereof. In some embodiments, the MS data comprises an attribute associated with the data acquisition method and/or an attribute of the mass spectrometer. In some embodiments, the MS data comprises the instrument type or a feature thereof. In some embodiments, the MS data comprises the degree of accuracy of the mass spectrometer on which the data was obtained, for example, high resolution data accuracy of an orbitrap mass spectrometer. In some embodiments, the MS data comprises the ion mode, such as positive ion mode or negative ion mode. In some embodiments, the MS data comprises the fragmentation technique, such as collision-induced dissociation (CID), surface-induced dissociation (SID), electron-capture dissociation (ECD), electron-transfer dissociation (ETD), negative electron-transfer dissociation (NETD), electron-detachment dissociation (EDD), photodissociation, infrared multiphoton dissociation (IRMPD), blackbody infrared radiative dissociation (BIRD), or higher-energy C-trap dissociation (HCD). In some embodiments, the MS data comprises a front-end mass spectrometry attribute, such as ion mobility.

In some embodiments, the mass spectrometry technique comprises an online or offline separation technique, such as liquid chromatography-mass spectrometry. In some embodiments, the MS data comprises an attribute associated with the separation technique, such as retention time and/or chromatography conditions.

The present invention contemplates a diverse array mass spectrometry techniques for generating MS data, such as fragmentation information from a tandem mass spectrum. In some embodiments, the mass spectrometry technique is a liquid chromatography-mass spectrometry technique. Liquid chromatography techniques contemplated by the present application include methods for separating compounds and liquid chromatography techniques compatible with mass spectrometry techniques. In some embodiments, the liquid chromatography technique comprises a high performance liquid chromatography technique. In some embodiments, the liquid chromatography technique comprises an ultra-high performance liquid chromatography technique. In some embodiments, the liquid chromatography technique comprises a high-flow liquid chromatography technique. In some embodiments, the liquid chromatography technique comprises a low-flow liquid chromatography technique, such as a micro-flow liquid chromatography technique or a nano-flow liquid chromatography technique. In some embodiments, the liquid chromatography technique comprises an online liquid chromatography technique coupled to a mass spectrometer. In some embodiments, the online liquid chromatography technique is a high performance liquid chromatography technique. In some embodiments, the online liquid chromatography technique is an ultra-high performance liquid chromatography technique. In some embodiments, capillary electrophoresis (CE) techniques, or electrospray or MALDI techniques may be used to introduce a compound to a mass spectrometer.

Mass spectrometry techniques comprise an ionization technique. Ionization techniques contemplated by the present application include techniques capable of charging compounds. Thus, in some embodiments, the ionization technique is electrospray ionization. In some embodiments, the ionization technique is nano-electrospray ionization. In some embodiments, the ionization technique is atmospheric pressure chemical ionization. In some embodiments, the ionization technique is atmospheric pressure photoionization. In some embodiments, the ionization technique is matrix-assisted laser desorption ionization (MALDI). In some embodiment, the mass spectrometry technique comprises electrospray ionization, nanoelectrospray ionization, or a matrix-assisted laser desorption ionization (MALDI) technique.

A plethora of mass spectrometers and techniques contemplated by the present invention include high-resolution mass spectrometers and low-resolution mass spectrometers.

In some embodiments, the mass spectrometer is a time-of-flight (TOF) mass spectrometer. In some embodiments, the mass spectrometer is a quadrupole time-of-flight (Q-TOF) mass spectrometer. In some embodiments, the mass spectrometer is a quadrupole ion trap time-of-flight (QIT-TOF) mass spectrometer. In some embodiments, the mass spectrometer is an ion trap. In some embodiments, the mass spectrometer is a single quadrupole. In some embodiments, the mass spectrometer is a triple quadrupole (QQQ). In some embodiments, the mass spectrometer is an orbitrap. In some embodiments, the mass spectrometer is a quadrupole orbitrap. In some embodiments, the mass spectrometer is a Fourier transform ion cyclotron resonance (FT) mass spectrometer. In some embodiments, the mass spectrometer is a quadrupole Fourier transform ion cyclotron resonance (Q-FT) mass spectrometer. In some embodiments, the mass spectrometry technique comprises positive ion mode. In some embodiments, the mass spectrometry technique comprises negative ion mode. In some embodiments, the mass spectrometry technique comprises a time-of-flight (TOF) mass spectrometry technique. In some embodiments, the mass spectrometry technique comprises a quadrupole time-of-flight (Q-TOF) mass spectrometry technique. In some embodiments, the mass spectrometry technique comprises an ion mobility mass spectrometry technique. In some embodiments a low-resolution mass spectrometry technique, such as an ion trap, or single or triple-quadrupole approach is appropriate.

In some embodiments, the compound is a small molecule, such as a natural or synthetic small molecule compound. In some embodiments, the small molecule is obtained or derived from a plant extract. In some embodiments, the small molecule is a therapeutic candidate, such as a candidate for use in treating a human disease or in the development of a therapeutic. In some embodiments, the compound has a molecular weight of less than 2,500 Da, such as 500 Da or less. In some embodiments, the compound satisfies one or more of Lipinski's rule of five. In some embodiments, the compound is a small molecule (such as a therapeutic small molecule that is 1,000 Da or less and/or satisfies one or more of Lipinski's rule of five). In some embodiments, the compound, or a portion thereof, is charged. In some embodiments, the compound, or a portion thereof, is hydrophobic. In some embodiments, the compound, or a portion thereof, is hydrophilic.

As used herein, “mass spectrometry data”, “MS data”, or “mass spectra data” may refer to, for example, one or more values or textual characters corresponding to a number of mass spectra charged fragments, a number of mass spectral intensities (e.g., a measure of abundance of the m/z peaks within MS fragmentation spectrum), a number of parent ion mass (e.g., the m/z value of the compound prior to fragmentation), or a retention time (e.g., compounds are eluted from LC to the MS and the time of elution is going to be correlated to some property of the compound).

### 2. Inference Phase of a Trained Bidirectional Transformer-Based Machine-Learning Model for Predicting Chemical Structures Utilizing Tokenizations of MS Data

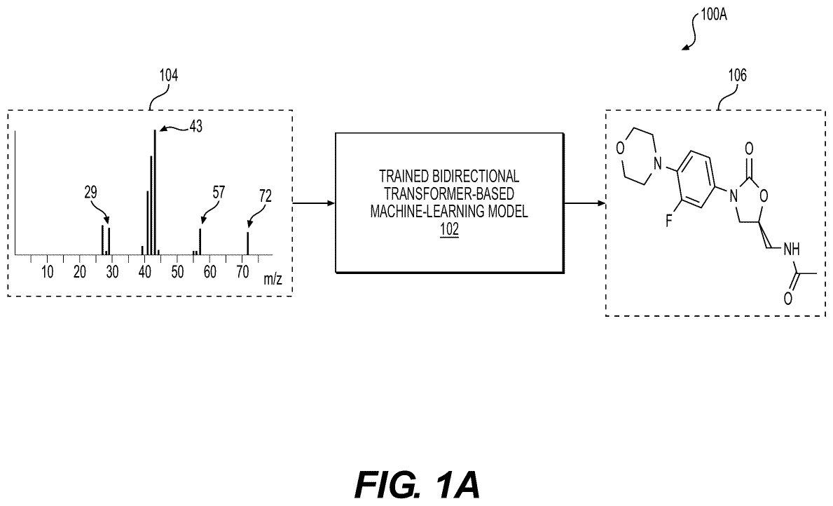

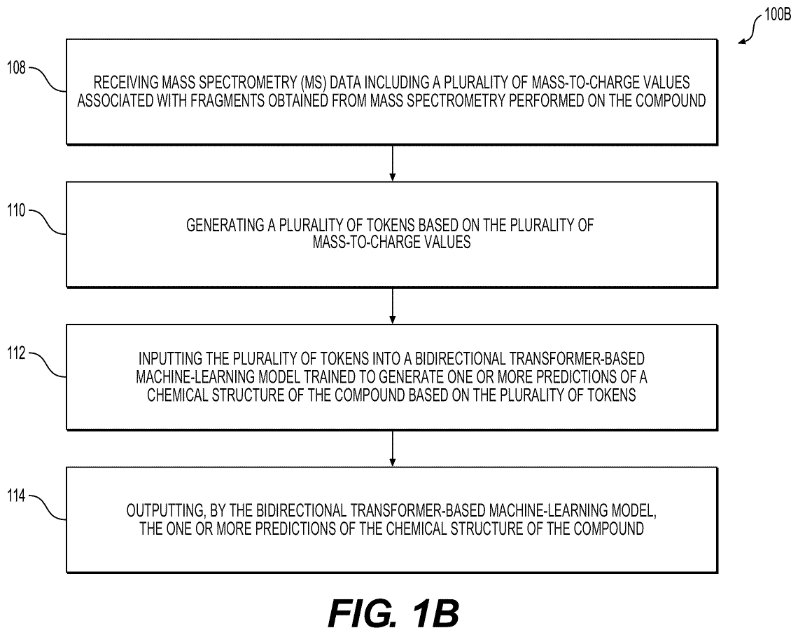

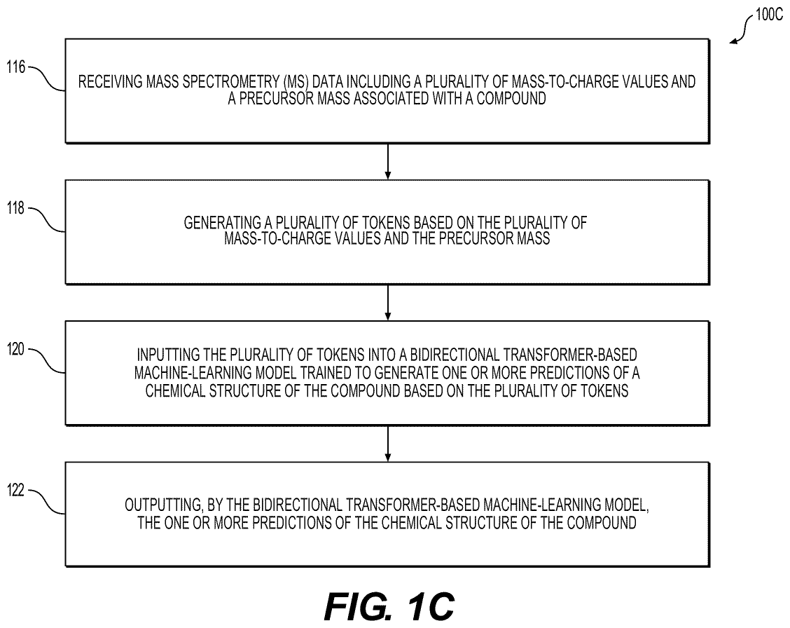

FIG. 1A illustrates an example embodiment of a workflow diagram 100A of an inference phase of a trained bidirectional transformer-based machine-learning model 102 for generating predictions of the chemical structure or chemical properties of molecules, compounds, and small molecules (e.g., metabolites) based on mass spectrometry MS data, in accordance with the presently disclosed embodiments. In certain embodiments, the workflow diagram 100A may begin with receiving or accessing MS data 104. In certain embodiments, the MS data 104 may include, for example, a data set of mass-to-charge (m/z) values associated with fragments obtained from mass spectrometry (e.g., MS, MS2, IM) performed on one or more naturally-occurring and/or non-naturally-occurring molecules, compounds, or small molecules (e.g., metabolites). In certain embodiments, the MS data 104 may be then inputted into the trained bidirectional transformer-based machine-learning model 102. In some embodiments, prior to inputting the MS data 104 into the trained bidirectional transformer-based machine-learning model 102, the MS data 104 may be encoded into one or more textual representations or vector representations and then the trained bidirectional transformer-based machine-learning model 102.

In one embodiment, the trained bidirectional transformer-based machine-learning model 102 may include, for example, a trained bidirectional and auto-regressive transformer (BART) model or one or more other natural language processing (NLP) models that may be suitable for translating the MS data 104 into one or more SMILEs strings representative of a predicted chemical structure of one or more naturally-occurring and/or non-naturally-occurring molecules, compounds, or small molecules (e.g., metabolites) corresponding to the MS data 104. In another embodiment, the trained bidirectional transformer-based machine-learning model 102 may include a bidirectional encoder representations for transformer (BERT) model, a generative pre-trained transformer (GPT) model, or some combination of a BERT model and a GPT model. In certain embodiments, as further depicted by FIG. 1A, based on the inputted MS data 104, the trained bidirectional transformer-based machine-learning model 102 may then output one or more SMILEs strings, DeepSMILES stings, or SELFIES strings representative of a predicted chemical structure 106 of one or more naturally-occurring and/or non-naturally-occurring molecules, compounds, or small molecules (e.g., metabolites) corresponding to the MS data 104.

FIG. 1B illustrates a flow diagram 100B of a method for generating predictions of the chemical structure or chemical properties of molecules, compounds, and small molecules (e.g., metabolites) based on mass spectrometry MS data, in accordance with the presently disclosed embodiments. The flow diagram 100B may be performed utilizing one or more processing devices (e.g., computational metabolomics computing system 500) that may include hardware (e.g., a general purpose processor, a graphic processing unit (GPU), an application-specific integrated circuit (ASIC), a system-on-chip (SoC), a microcontroller, a field-programmable gate array (FPGA), a central processing unit (CPU), an application processor (AP), a visual processing unit (VPU), a neural processing unit (NPU), a neural decision processor (NDP), a deep learning processor (DLP), or any other processing device(s) that may be suitable for processing genomics data, metabolomics data, proteomics data, metagenomics data, transcriptomics data, and/or various other omics data), software (e.g., instructions running/executing on one or more processors), firmware (e.g., microcode), or some combination thereof.

The flow diagram 100B may begin at block 108 with the one or more processing devices receiving MS data including a plurality of mass-to-charge values associated with fragments obtained from mass spectrometry performed on the compound. The flow diagram 100B may then continue at block 110 with the one or more processing devices generating a plurality of tokens based on the plurality of mass-to-charge values. The flow diagram 100B may then continue at block 112 with the one or more processing devices inputting the plurality of tokens into a bidirectional transformer-based machine-learning model trained to generate one or more predictions of a chemical structure of the compound based on the plurality of tokens. The flow diagram 100B may then conclude at block 114 with the one or more processing devices outputting, by the bidirectional transformer-based machine-learning model, the one or more predictions of the chemical structure of the compound.

FIG. 1C illustrates a flow diagram 100C of a method for generating predictions of the chemical structure or chemical properties of molecules, compounds, and small molecules (e.g., metabolites) based on mass spectrometry MS data including precursor mass, in accordance with the presently disclosed embodiments. The flow diagram 100C may be performed utilizing one or more processing devices (e.g., computational metabolomics computing system 500) that may include hardware (e.g., a general purpose processor, a graphic processing unit (GPU), an application-specific integrated circuit (ASIC), a system-on-chip (SoC), a microcontroller, a field-programmable gate array (FPGA), a central processing unit (CPU), an application processor (AP), a visual processing unit (VPU), a neural processing unit (NPU), a neural decision processor (NDP), a deep learning processor (DLP), or any other processing device(s) that may be suitable for processing genomics data, metabolomics data, proteomics data, metagenomics data, transcriptomics data, and/or various other omics data), software (e.g., instructions running/executing on one or more processors), firmware (e.g., microcode), or some combination thereof.

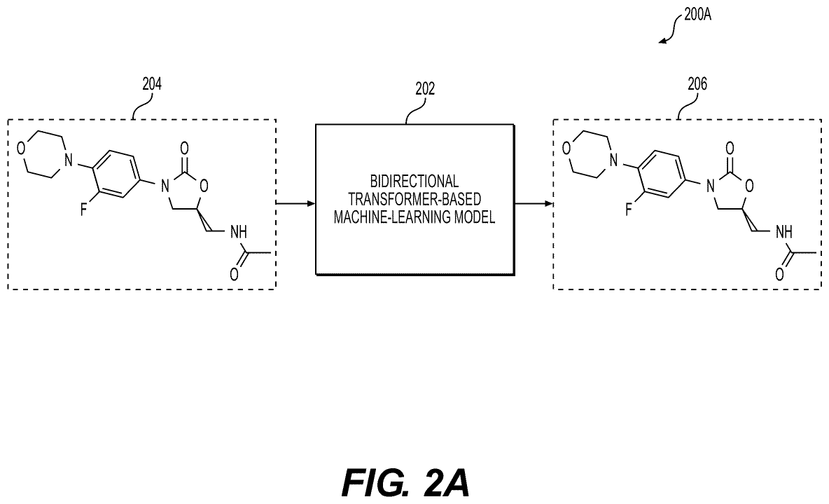

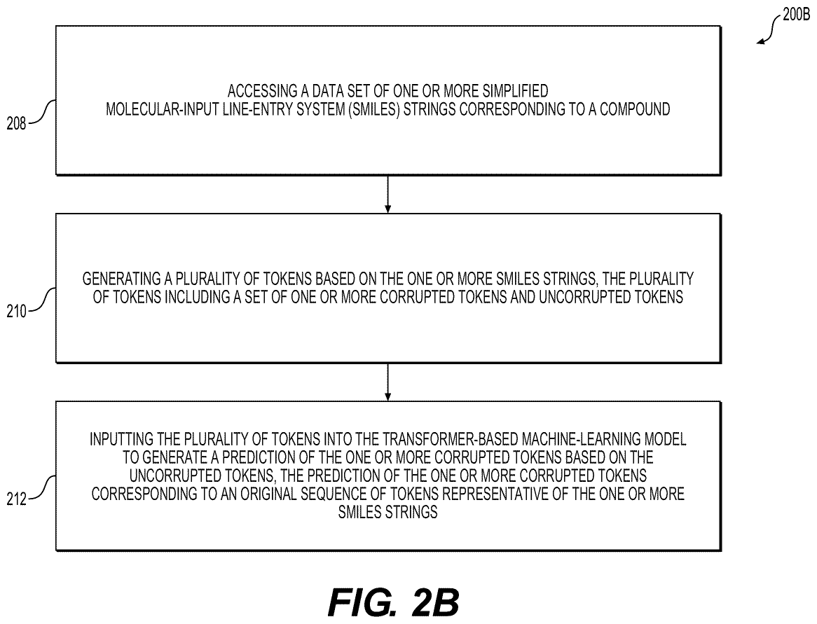

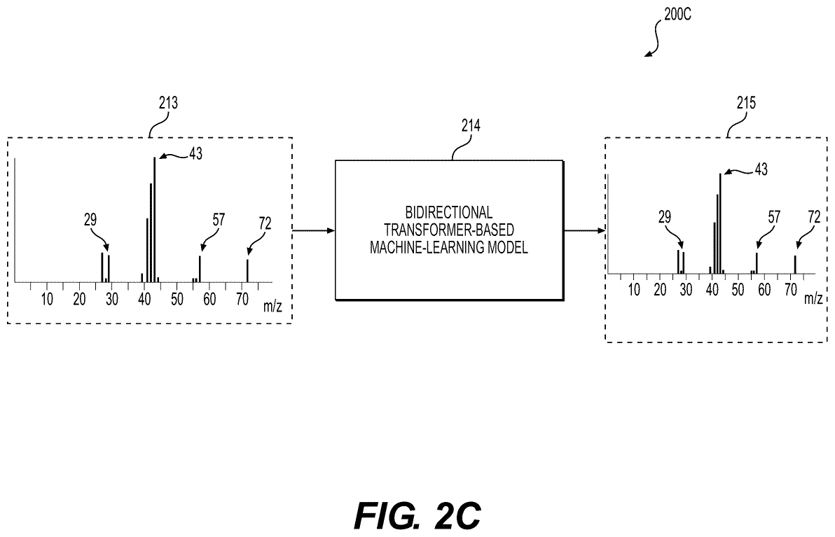

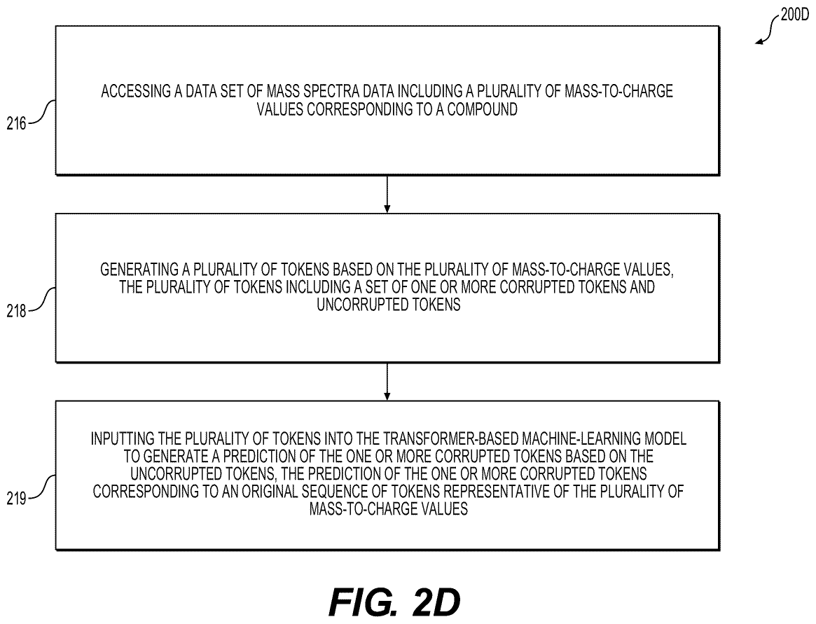

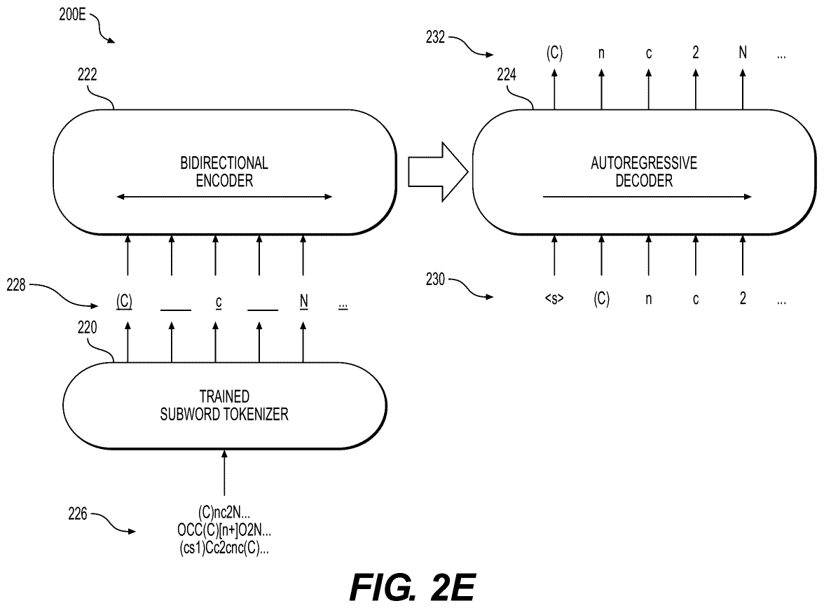

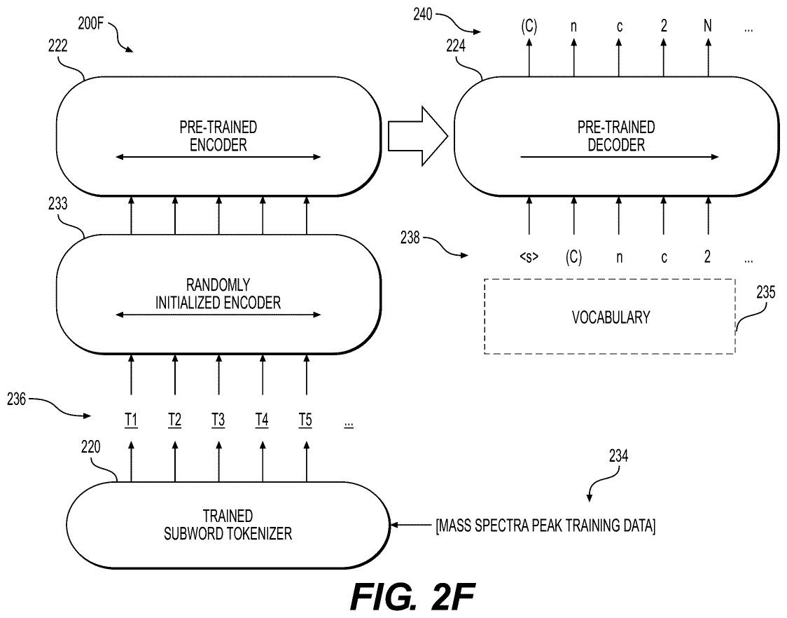

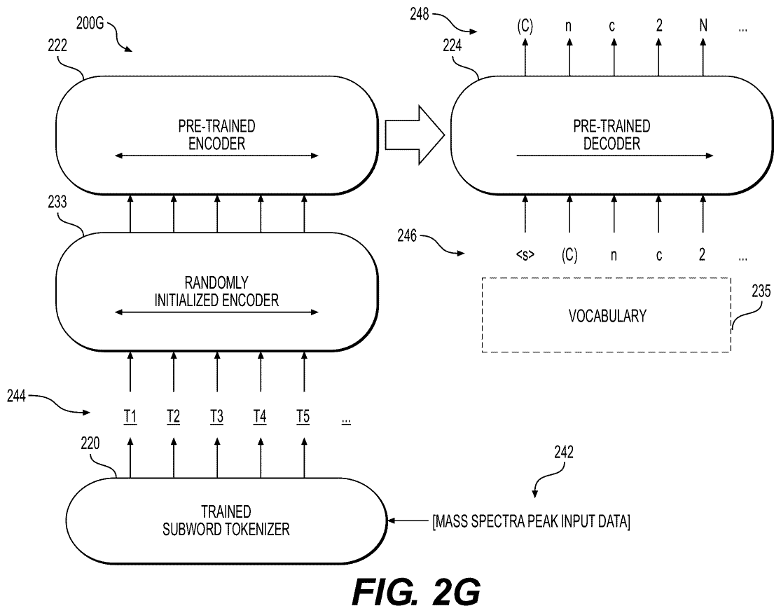

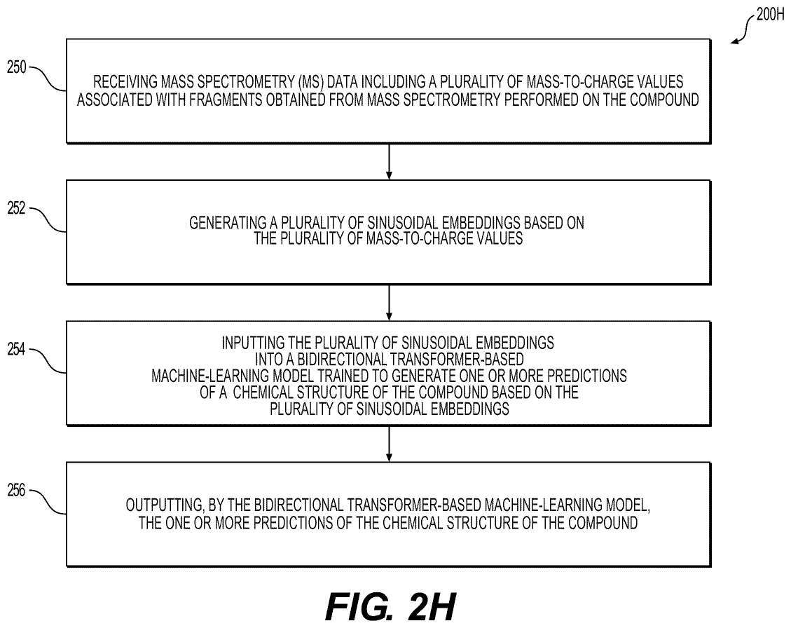

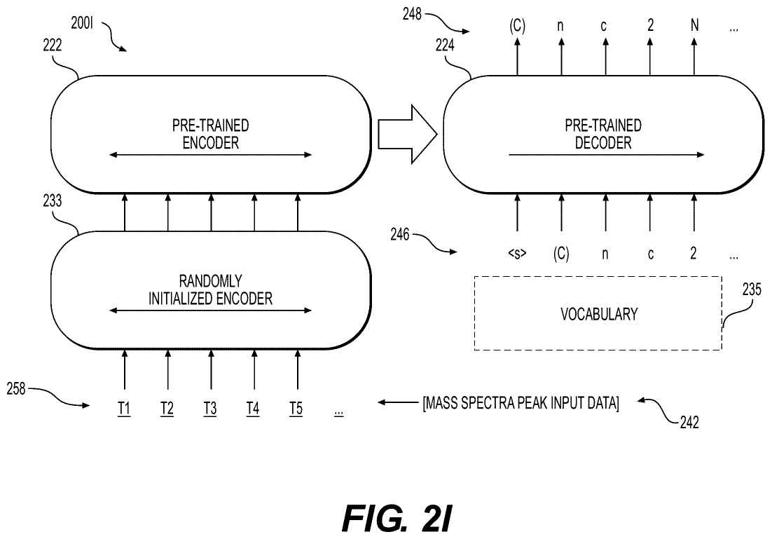

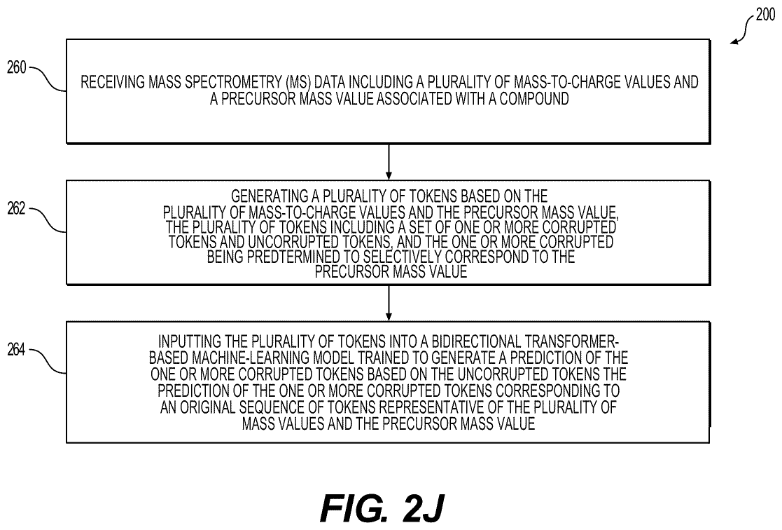

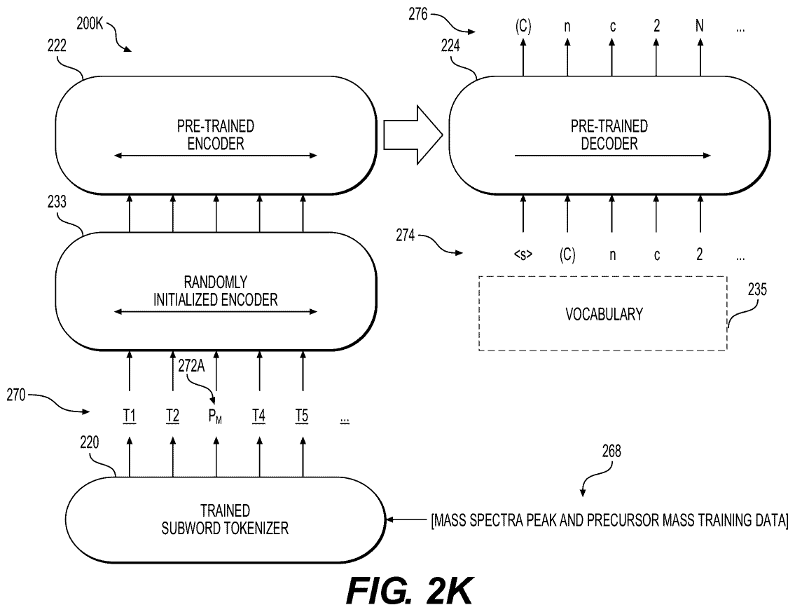

Specifically, in certain embodiments, in addition to inputting the MS data 104 (e.g., representing mass spectra peaks) into the trained bidirectional transformer-based machine-learning model 102, the trained bidirectional transformer-based machine-learning model 102 may also receive a precursor mass (e.g., precursor m/z). For example, in some embodiments, the precursor mass (e.g., precursor m/z) may represent the mass of, for example, an un-fragmented one or more naturally-occurring and/or non-naturally-occurring molecules, compounds, or small molecules (e.g., metabolites) corresponding to the MS data 104. In one embodiment, as will be further appreciated with respect to FIGS. 2J-2L, including the input of the precursor mass (e.g., precursor m/z) to the trained bidirectional transformer-based machine-learning model 102 may improve the ability of the bidirectional transformer-based machine-learning model to accurately predict the chemical structure of a compound (e.g., as compared to the mass spectra peak data of the MS data 104 alone).

Accordingly, the flow diagram 100C may begin at block 116 with the one or more processing devices receiving MS data including a plurality of mass-to-charge values and a precursor mass associated with a compound. The flow diagram 100C may then continue at block 118 with the one or more processing devices generating a plurality of tokens based on the plurality of mass-to-charge values and the precursor mass. The flow diagram 100C may then continue at block 120 with the one or more processing devices inputting the plurality of tokens into a bidirectional transformer-based machine-learning model trained to generate one or more predictions of a chemical structure of the compound based on the plurality of tokens. The flow diagram 100C may then conclude at block 122 with the one or more processing devices outputting, by the bidirectional transformer-based machine-learning model, the one or more predictions of the chemical structure of the compound.

### 3. Pre-Training and Fine-Tuning a Bidirectional Transformer-Based Machine-Learning Model for Predicting Chemical Structures Utilizing SMILES Strings

FIG. 2A illustrates an example embodiment of a workflow diagram 200A of a training phase for pre-training and fine-tuning a bidirectional transformer-based machine-learning model 202 for generating predictions of the chemical structure or chemical properties of molecules, compounds, and small molecules (e.g., metabolites) utilizing SMILES strings, in accordance with the presently disclosed embodiments. In certain embodiments, the workflow diagram 200A may begin with receiving or accessing a data set of one or more SMILES strings representative of an original chemical structure 204 corresponding to one or more molecules, compounds, and small molecules (e.g., metabolites). In one embodiment, the data set of one or more SMILES strings representative of an original chemical structure 204 may include, for example, unlabeled data corresponding to one or more naturally-occurring molecules, compounds, and small molecules (e.g., metabolites). In some embodiments the input structure may include masking of parts of the chemical structure.

In certain embodiments, the data set of one or more SMILES strings representative of an original chemical structure 204 may be then inputted into the bidirectional transformer-based machine-learning model 202. In one embodiment, the bidirectional transformer-based machine-learning model 202 may include, for example, a BART model or one or more other NLP models that may be pre-trained and fine-tuned for translating MS data into one or more SMILEs strings representative of a predicted chemical structure of one or more naturally-occurring and/or non-naturally-occurring molecules, compounds, or small molecules (e.g., metabolites). In another embodiment, the bidirectional transformer-based machine-learning model 202 may include a BERT model, a GPT model, or some combination of a BERT model and a GPT model.

In certain embodiments, during the pre-training phase, the bidirectional transformer-based machine-learning model 202 may be pre-trained to learn broad and granular patterns in the data set of one or more SMILES strings representative of an original chemical structure 204 before being fine-tuned to translate (e.g., machine translation) MS data into SMILES strings representative of one or more predicted chemical structures 206 (e.g., equivalent to pre-training the bidirectional transformer-based machine-learning model 202 to be proficient at the English language before fine-tuning the bidirectional transformer-based machine-learning model 202 to translate English language to the Spanish language). In certain embodiments, during the pre-training phase, one or more tokens of each SMILES string of the data set of one or more SMILES strings representative of an original chemical structure 204 may be corrupted and fed to the bidirectional transformer-based machine-learning model 202. The bidirectional transformer-based machine-learning model 202 may then attempt to predict the full sequence of tokens of the respective SMILES string based on the one or more uncorrupted tokens of the sequence of tokens of the respective SMILES string.

In certain embodiments, the one or more tokens of each SMILES string may be corrupted, for example, utilizing a token deletion process, a token masking process, a text infilling process, a text string permutation process, or a sequence rotation process. In certain embodiments, a sequence of tokens of each SMILES string including the one or more corrupted tokens and the uncorrupted tokens may be then inputted into the transformer-based machine-learning model 202 to generate a prediction of the one or more corrupted tokens based on the uncorrupted tokens. In certain embodiments, the bidirectional transformer-based machine-learning model 202 may then output the prediction of the one or more corrupted tokens based on the uncorrupted tokens, in which the prediction may include one or more SMILES strings representative of one or more predicted chemical structures 206.

In certain embodiments, transformer-based machine-learning model 202 may be then further pre-trained by computing a cross-entropy loss value based on a comparison of the prediction of the SMILES strings representative of one or more predicted chemical structures 206 and the one or more SMILES strings representative of the original chemical structure 204, and updating the transformer-based machine-learning model 202 based on the cross-entropy loss value. In certain embodiments, the pre-trained transformer-based machine-learning model 202 may be fine-tuned by accessing a data set of MS data 104, for example, inputting the data set of MS data 104 into the pre-trained transformer-based machine-learning model 202, and generating one or more SMILES strings representative of the one or more predicted chemical structures 206. In certain embodiments, the fine-tuned transformer-based machine-learning model 202 may be then further fine-tuned by computing a second cross-entropy loss value based on a comparison of the one or more SMILES strings representative of the one or more predicted chemical structures 206 and an original sequence of tokens representative of the MS data 104, for example, and updating the fine-tuned transformer-based machine-learning model 202 based on the second cross-entropy loss value.

FIG. 2B illustrates a flow diagram 200B of a method for pre-training and fine-tuning a bidirectional transformer-based machine-learning model to generate predictions of the chemical structure of a compound utilizing SMILES strings, in accordance with the presently disclosed embodiments. The flow diagram 200B may be performed utilizing one or more processing devices (e.g., computational metabolomics computing system 500) that may include hardware (e.g., a general purpose processor, a graphic processing unit (GPU), an application-specific integrated circuit (ASIC), a system-on-chip (SoC), a microcontroller, a field-programmable gate array (FPGA), a central processing unit (CPU), an application processor (AP), a visual processing unit (VPU), a neural processing unit (NPU), a neural decision processor (NDP), a deep learning processor (DLP), a tensor processing unit (TPU), or any other processing device(s) that may be suitable for processing genomics data, metabolomics data, proteomics data, metagenomics data, transcriptomics data, and/or various other omics data), software (e.g., instructions running/executing on one or more processors), firmware (e.g., microcode), or some combination thereof.

The flow diagram 200B may begin at block 208 with the one or more processing devices accessing a data set of one or more SMILES strings corresponding to a compound. The flow diagram 200B may then continue at block 210 with the one or more processing devices generating a plurality of tokens based on the one or more SMILES strings, the plurality of tokens including a set of one or more corrupted tokens and uncorrupted tokens. The flow diagram 200B may then conclude at block 212 with the one or more processing devices inputting the plurality of tokens into the transformer-based machine-learning model to generate a prediction of the one or more corrupted tokens based on the uncorrupted tokens, in which the prediction of the one or more corrupted tokens corresponds to an original sequence of tokens representative of the one or more SMILES strings.

### 4. Pre-Training and Fine-Tuning a Bidirectional Transformer-Based Machine-Learning Model for Predicting Chemical Structures Utilizing Tokenizations of MS Data

FIG. 2C illustrates an example embodiment of a workflow diagram 200C of a training phase for pre-training and fine-tuning a bidirectional transformer-based machine-learning model 202 for generating predictions of the chemical structure of molecules, compounds, and small molecules (e.g., metabolites) utilizing MS data, in accordance with the presently disclosed embodiments. In certain embodiments, the workflow diagram 200C may begin with receiving or accessing a data set of MS data 213 corresponding to one or more molecules, compounds, and small molecules (e.g., metabolites). In one embodiment, the data set of MS data 213 may include, for example, unlabeled data corresponding to one or more naturally-occurring molecules, compounds, and small molecules (e.g., metabolites). In certain embodiments, the data set of MS data 213 may be then inputted into the bidirectional transformer-based machine-learning model 202.

In certain embodiments, prior to the data set of MS data 213 may being inputted into the bidirectional transformer-based machine-learning model 202, the MS data 213 may be encoded into one or more text strings or vector representations of mass-to-charge values and then tokenized. In one embodiment, the MS data 213 may be tokenized by clustering (e.g., hierarchical clustering, k-means clustering, and so forth), for example, in 2 dimensions, in which the 2 dimensions represent the integer value of a mass-to-charge (m/z) fragment and the fractional value of the mass-to-charge (m/z) fragment, respectively. In another embodiment, the MS data 213 may be tokenize by binning the mass-to-charge (m/z) fragments in accordance with one or more precision values.

In certain embodiments, during the pre-training phase, the bidirectional transformer-based machine-learning model 202 may be pre-trained to learn broad and granular patterns in the data set of MS data 213 before being fine-tuned to translate (e.g., machine translation) the MS data 213 into SMILES strings representative of one or more predicted chemical structures (e.g., equivalent to pre-training the bidirectional transformer-based machine-learning model 202 to be proficient at the English language before fine-tuning the bidirectional transformer-based machine-learning model 202 to translate English language to the Spanish language as previously discussed above with respect to FIG. 2A). In certain embodiments, during the pre-training phase, one or more tokens of a text string (e.g., a vector representation of mass-to-charge values) representative of the data set of MS data 213 may be corrupted and fed to the bidirectional transformer-based machine-learning model 202. The bidirectional transformer-based machine-learning model 202 may then attempt to predict the full sequence of tokens of the one or more text strings (e.g., one or more vector representations of mass-to-charge values) representative of the data set of MS data 213 based on the one or more uncorrupted tokens of the sequence of tokens of the text string (e.g., a vector representation of mass-to-charge values) representative of the data set of MS data 213.

In certain embodiments, the one or more tokens of one or more text strings (e.g., one or more vector representations) representative of the data set of MS data 213 may be corrupted, for example, utilizing a token deletion process, a token masking process, a text infilling process, a text string permutation process, or a sequence rotation process. In certain embodiments, a sequence of tokens of the text string including the one or more corrupted tokens and the uncorrupted tokens may be then inputted into the transformer-based machine-learning model 202 to generate a prediction of the one or more corrupted tokens based on the uncorrupted tokens. In certain embodiments, the bidirectional transformer-based machine-learning model 202 may then output the prediction of the one or more corrupted tokens based on the uncorrupted tokens, in which the prediction may include a text string (e.g., a vector representation) corresponding to the one or more text strings (e.g., one or more vector representations) representative of the data set of MS data 213.

In certain embodiments, transformer-based machine-learning model 202 may be then further pre-trained by computing a cross-entropy loss value based on a comparison of the predicted text string (e.g., a vector representation of mass-to-charge values) and the one or more text strings (e.g., one or more vector representations of mass-to-charge values) representative of the data set of MS data 213, and updating the transformer-based machine-learning model 202 based on the cross-entropy loss value. In certain embodiments, the pre-trained transformer-based machine-learning model 202 may be fine-tuned by accessing a the data set of MS data 213, for example, and inputting the data set of MS data 213 into the pre-trained transformer-based machine-learning model 202 to generate one or more SMILES strings representative of a predicted chemical structure of one or more molecules, compounds, or small molecules (e.g., metabolites) corresponding to the data set of MS data 213. In certain embodiments, the fine-tuned transformer-based machine-learning model 202 may be then further fine-tuned by computing a second cross-entropy loss value based on a comparison of the one or more SMILES strings representative of the one or more predicted chemical structures and an original sequence of tokens representative of data set of MS data 213, for example, and updating the fine-tuned transformer-based machine-learning model 202 based on the second cross-entropy loss value.

In some embodiments, for predicting chemical structure and/or chemical properties of the MS data 104, each training iteration or instance may include one MS/MS2 fragmentation spectrum. In some embodiments, each training iteration or instance may be given equal weight (e.g., unweighted) with respect to the total loss value of the transformer-based machine-learning model 202. However, in some embodiments, multiple MS/MS2 spectra may be gathered together for a single molecule, compound, or small molecule (e.g., metabolites), and the number of MS/MS2 spectra per molecule, compound, or small molecule may regularly vary. Thus, equally weighting of the loss value (e.g., unweighted loss) may lead to the transformer-based machine-learning model 202 prioritizing learning well only those molecules, compounds, and small molecules (e.g., metabolites) for which there are a large number of MS/MS2 spectra as compared to other molecules, compounds, and small molecules (e.g., metabolites) for which there are only a small number of MS/MS2 spectra, for example.

Accordingly, in certain embodiments, it may be useful to assign a weighting to each training iteration or instance with respect to the total loss of the transformer-based machine-learning model 202. For example, in some embodiments, the weighting assigned to each training iteration or instance loss may be the inverse of the number of MS/MS2 spectra. In this way, each molecule, compound, or small molecule may be assigned equal weighting with respect to the transformer-based machine-learning model 202 as opposed to assigning equal weighting to each MS2 fragmentation spectrum, for example. In one embodiment, the weighted loss function may include a weighted cross-entropy loss function. In one embodiment, the weighted cross-entropy loss function may be expressed as:

$$ℒ = {\sum }_{S ∈ Structures} \frac{1}{\vert MS ( S ) + κ \vert} {\sum }_{t ∈ MS ( S )} log ( p ( S \vert t ) ) )$$

In certain embodiments, referring to the weighted loss function above, K is a regularization parameter that may be preselected, in which K=0 may be complete weighting. In certain embodiments, the limit as K increases may be equivalent to an equally weighted loss (e.g., unweighted loss). Thus, in one embodiment, K may be preselected to be a value 1. Referring again to the weighted loss function above, MS(S) may be the set of MS/MS2 spectra associated with structure S. It should be appreciated that the foregoing examples described with respect to the weighted loss function may represent only one embodiment of the presently disclosed techniques of assigning a weighting to each training iteration or instance with respect to the total loss of the transformer-based machine-learning model 202. In other embodiments, various elaborations may be performed based on the weighted loss function, such as exponentiating the MS(S)+K term with different exponents, for example.

FIG. 2D illustrates a flow diagram 200D of a method for pre-training and fine-tuning a bidirectional transformer-based machine-learning model to generate predictions of the chemical structure of a compound utilizing MS data, in accordance with the presently disclosed embodiments. The flow diagram 200D may be performed utilizing one or more processing devices (e.g., computational metabolomics computing system 500) that may include hardware (e.g., a general purpose processor, a graphic processing unit (GPU), an application-specific integrated circuit (ASIC), a system-on-chip (SoC), a microcontroller, a field-programmable gate array (FPGA), a central processing unit (CPU), an application processor (AP), a visual processing unit (VPU), a neural processing unit (NPU), a neural decision processor (NDP), a deep learning processor (DLP), a tensor processing unit (TPU), or any other processing device(s) that may be suitable for processing genomics data, metabolomics data, proteomics data, metagenomics data, transcriptomics data, and/or various other omics data), software (e.g., instructions running/executing on one or more processors), firmware (e.g., microcode), or some combination thereof.

The flow diagram 200D may begin at block 216 with the one or more processing devices accessing a data set of mass spectra data including a plurality of mass-to-charge values corresponding to a compound. The flow diagram 200B may then continue at block 218 with the one or more processing devices generating a plurality of tokens based on the plurality of mass-to-charge values, the plurality of tokens including a set of one or more corrupted tokens and uncorrupted tokens. The flow diagram 200B may then conclude at block 219 with the one or more processing devices inputting the plurality of tokens into the transformer-based machine-learning model to generate a prediction of the one or more corrupted tokens based on the uncorrupted tokens, in which the prediction of the one or more corrupted tokens corresponding to an original sequence of tokens representative of the plurality of mass-to-charge values.

### 5. Running Examples of Pre-Training and Fine-Tuning a Bidirectional Transformer-Based Machine-Learning Model for Predicting Chemical Structures Utilizing Tokenizations of MS Data

FIGS. 2E and 2F illustrate one or more running examples 200E and 200F for pre-training and fine-tuning a bidirectional transformer-based machine-learning model to generate predictions of the chemical structure of a compound, in accordance with the presently disclosed embodiments. For example, in some embodiments, the one or more running examples 200E and 200F may be illustrated with respect to a bidirectional transformer-based machine-learning model, which may include a bidirectional encoder 222 and an autoregressive decoder 224. For example, in one embodiment, the bidirectional encoder 222 may include a BERT model and the autoregressive decoder 224 may include a GPT model that may operate, for example, in conjunction. In certain embodiments, the bidirectional encoder 222 and the autoregressive decoder 224 may be each associated with a trained subword tokenizer 220 (e.g., BPE tokenizer, WordPiece tokenizer, Unigram tokenizer, BPE dropout tokenizer, and so forth).

In certain embodiments, as depicted by FIG. 2E, during the pre-training phase of the bidirectional encoder 222 (e.g., BERT model) and the autoregressive decoder 224 (e.g., GPT model), the trained subword tokenizer 220 (e.g., BPE tokenizer, WordPiece tokenizer, Unigram tokenizer, BPE dropout tokenizer, and so forth) may receive one or more textual strings 226. In certain embodiments, the one or more textual strings 226 may include, for example, one or more SMILES strings, DeepSMILES strings, SELFIES strings, or other similar textual representations of compounds, molecules, or small molecule (e.g., metabolites). The trained subword tokenizer 220 may then tokenize one or more textual strings 226 (e.g., SMILES string “(C)nc2N . . . . . . . . . ”) into a sequence of tokens 228 (e.g., “(C)”, “n”, “c”, “2” “N”, “ . . . ” (e.g., equivalent to deconstructing a sentence into individual phrases or individual words)).

In certain embodiments, as further depicted, a token corrupting process may be then performed to mask or corrupt one or more of the sequence of tokens 228 (e.g., “(C)”, “n”, “c”, “2” “N”, “ . . . ”) to generate a sequence of corrupted and uncorrupted tokens 228 (e.g., “(C)”, “_”, “c”, “_” “N”, “ . . . ”). In certain embodiments, the sequence of corrupted and uncorrupted tokens 228 (e.g., “(C)”, “_”, “c”, “_” “N”, “ . . . ”) may be then inputted into the bidirectional encoder 222 (e.g., BERT model) to train the bidirectional encoder 222 (e.g., BERT model) and the autoregressive decoder 224 (e.g., GPT model) to generate an output sequence of tokens 232 (e.g., “(C)”, “n”, “c”, “2” “N”, “ . . . ”) corresponding to the original uncorrupted sequence of tokens 228 (e.g., “(C)”, “n”, “c”, “2” “N”, “ . . . ”). In one embodiment, the output sequence of tokens 232 (e.g., “(C)”, “n”, “c”, “2” “N”, “ . . . ”) prediction may include one or more SMILES strings representative of one or more predicted chemical structures.

For example, in certain embodiment, the bidirectional encoder 222 (e.g., BERT model) may receive the sequence of corrupted and uncorrupted tokens 228 (e.g., “(C)”, “_”, “c”, “_” “N”, “ . . . ”) and generate an output to be provided to the autoregressive decoder 224 (e.g., GPT model). For example, in one embodiment, the bidirectional encoder 222 (e.g., BERT model) may generate the output by performing, for example, a masked language modeling (MLM) “fill-in-the-blank” process to attempt to predict the one or more corrupted tokens (e.g., “_”, “_”) based on the one or more uncorrupted tokens (e.g., “(C)”, “c”, “N”, “ . . . ”). The autoregressive decoder 224 (e.g., GPT model) may then receive a sequence of tokens 230 (e.g., “<S>”, “(C)”, “n”, “c”, “2”) including a start-of-sequence token, and utilize the sequence of tokens 230 (e.g., “<S>”, “(C)”, “n”, “c”, “2”) and the output from the bidirectional encoder 222 (e.g., BERT model) to generate an output sequence of tokens 232 (e.g., “(C)”, “n”. “c”, “2” “N”, “ . . . ”) corresponding to the original uncorrupted sequence of tokens 228 (e.g., “(C)”, “n”, “c”, “2” “N”, “ . . . ”). For example, in one embodiment, the autoregressive decoder 224 (e.g., GPT model) may generate the output by performing, for example, one or more autoregressive processes to attempt to predict and generate the next token (e.g., “N”) based on the sequence of tokens 230 (e.g., “<S>”, “(C)”, “n”, “c”, “2”) and the output from the bidirectional encoder 222 (e.g., BERT model).

In certain embodiments, as depicted by FIG. 2F, during the fine-tuning phase of the bidirectional encoder 222 (e.g., BERT model) and the autoregressive decoder 224 (e.g., GPT model), the trained subword tokenizer 220 (e.g., BPE tokenizer, WordPiece tokenizer, Unigram tokenizer, BPE dropout tokenizer, and so forth) may receive MS training data 234 and generate a sequence of tokens 236 (e.g., “T1”, “T2”, “T3”, “T4” “T5”, “ . . . ”). In one embodiment, the sequence of tokens 236 (e.g., “T1”, “T2”, “T3”, “T4” “T5”, “ . . . ”) may represent one or more text strings or vector representations corresponding to, for example, a data set of mass spectral peaks derived from the MS training data 234. In certain embodiments, as further depicted by FIG. 2F, the trained subword tokenizer 220 may output the sequence of tokens 236 (e.g., “T1”, “T2”, “T3”, “T4” “T5”, “ . . . ”) into a randomly initialized encoder 233 (e.g., NLP model) that may be suitable for learning contextual data (e.g., positional encodings and embeddings) based on the sequence of tokens 236 (e.g., “T1”, “T2”, “T3”, “T4” “T5”, “ . . . ”). It should be appreciated that the running example 200E may represent only one embodiment of the bidirectional transformer-based machine-learning model. For example, in other embodiments, the randomly initialized encoder 233 (e.g., NLP model) may not be included as part of bidirectional transformer-based machine-learning model architecture. Thus, in such embodiments, the trained subword tokenizer 220 may output the sequence of tokens 236 (e.g., “T1”, “T2”, “T3”, “T4” “T5”, “ . . . ”) directly to the bidirectional encoder 222 (e.g., BERT model).

Particularly, as used herein, the “embeddings layer” may refer to one of an input embedding layer to, for example, the randomly initialized encoder 233 and/or bidirectional encoder 222 (e.g., BERT model) or an output embedding layer to, for example, the autoregressive decoder 224 (e.g., GPT model). For example, the “embedding layer” may be utilized to encode the meaning of each token of the input sequence of tokens 236 (e.g., “T1”, “T2”, “T3”, “T4” “T5”, “ . . . ”) in accordance with the context of the MS training data 234 and/or the MS input data 242. Similarly, as used herein, the “position encoding layer” may refer to one of an input positional encoding layer to, for example, the randomly initialized encoder 233 and/or bidirectional encoder 222 (e.g., BERT model) or an output positional encoding layer to, for example, the autoregressive decoder 224 (e.g., GPT model). For example, the “positional encoding layer” may be utilized to encode the position of each token of the input sequence of tokens 236 (e.g., “T1”, “T2”, “T3”, “T4” “T5”, “ . . . ”) in accordance with the context of the MS training data 234 and/or the MS input data 242. Indeed, in accordance with the presently disclosed embodiments, any of the bidirectional transformer-based machine-learning models may include one or more of an input embedding layer, an output embedding layer, an input position encoding layer, and an output embedding layer that may be utilized to encode the meaning and position of each token of the input sequence of tokens 236 (e.g., “T1”, “T2”, “T3”, “T4” “T5”, “ . . . ”) and/or the meaning and position of each token of the output sequence of tokens 232 (e.g., “(C)”, “n”, “c”, “2” “N”, “ . . . ”) in accordance with the context of the MS data 234 and/or the MS input data 242. In some embodiments, as discussed in greater detail below, the position encoding layer may be utilized to encode the MS training data 234 and/or the MS input data 242 as a sequence of mass-to-charge values ordered from least intensity to greatest intensity, or vice-versa.

In certain embodiments, the bidirectional encoder 222 (e.g., BERT model), the autoregressive decoder 224 (e.g., GPT model), and the randomly initialized encoder 233 may each be associated with a vocabulary 235. In certain embodiments, the vocabulary 235 may include any library including various individual characters, words, subwords, sequences of numerical values, sequences of sequential characters, sequences of sequential numerical values, and so forth that may be augmented and updated over time. In some embodiments, the vocabulary 235 may be accessed by the bidirectional encoder 222 (e.g., BERT model), the autoregressive decoder 224 (e.g., GPT model), and the randomly initialized encoder 233 during the pre-training phase and/or fine-tuning phase. In another embodiment, each of the bidirectional encoder 222 (e.g., BERT model), the autoregressive decoder 224 (e.g., GPT model), and the randomly initialized encoder 233 may be associated with its own vocabulary 235.

In certain embodiments, the randomly initialized encoder 233 (e.g., NLP model) may then generate an output that may be received by the bidirectional encoder 222 (e.g., BERT model). The bidirectional encoder 222 (e.g., BERT model) and the autoregressive decoder 224 (e.g., GPT model) may then proceed as discussed above with respect to FIG. 2E to translate (e.g., machine translation) the sequence of tokens 236 (e.g., “T1”, “T2”, “T3”, “T4” “T5”, “ . . . ”) into a prediction of an output sequence of tokens 240 (e.g., “(C)”, “n”, “c”, “2” “N”, “ . . . ”) corresponding to a machine translation of the sequence of tokens 236 (e.g., “T1”, “T2”, “T3”, “T4” “T5”, “ . . . ”) representing a data set of mass spectral peaks derived from the MS training data 234 into a prediction of an output sequence of tokens 240 (e.g., “(C)”, “n”, “c”, “2” “N”, “ . . . ”) corresponding to one or more SMILES strings or other similar textual representations of compounds, molecules, or small molecules (e.g., metabolites).

In certain embodiments, in addition to, or alternative to the forgoing techniques, the bidirectional encoder 222 (e.g., BERT model) and the autoregressive decoder 224 (e.g., GPT model) may be further trained based on predetermined chemical data (e.g., a chemical formula, a representation of a chemical structural property), and may be utilized to infer additional data with respect to predicting one or more chemical structures based on MS data. For example, in one embodiment, the predetermined chemical data (e.g., a chemical formula, a representation of a chemical structural property) may include a start-of-sequence token for contextualizing one or more tokens to be generated based on a number of mass-to-charge values. In certain embodiments, the bidirectional encoder 222 (e.g., BERT model) may be further trained based on the sequence of tokens 228 (e.g., “(C)”, “n”, “c”, “2” “N”, “ . . . ”) and the associated predetermined chemical data (e.g., a chemical formula, a representation of a chemical structural property). For example, in one embodiment, a chemical formula or molecular weight may be encoded as a start-of-sequence token (e.g., “<S>”) and included in the input sequence of tokens 228 (e.g., “<S>”, “(C)”, “n”, “c”, “2” “N”, “ . . . ”).

In another embodiment, the chemical formula or molecular weight may be encoded as part of the positional layer encoding and/or embeddings layer encoding of the bidirectional encoder 222 (e.g., BERT model). In certain embodiments, the input sequence of tokens 228 (e.g., “<S>”, “(C)”, “n”, “c”, “2” “N”, “ . . . ”) including the start-of-sequence token (e.g., “<S>”) may be inputted to the bidirectional encoder 222 (e.g., BERT model) to generate a prediction based on the input sequence of tokens 228 (e.g., “<S>”, “(C)”, “n”, “c”, “2” “N”, “ . . . ”) and the predetermined chemical data (e.g., a chemical formula, a representation of a chemical structural property). In this way, the bidirectional encoder 222 (e.g., BERT model) may allow further inferences to be drawn from the MS training data 234. For example, for precise compound mass measurements, certain compounds may be inferred based on the bidirectional encoder 222 (e.g., BERT model) having learned chemical formula or other chemical data in addition to the MS data (e.g. C2H4 will always way be exactly 28.05 g, so 28.05 g is likely to indicate a C2H4 compound).

In other embodiments, the MS training data 234 may include a sequence of mass-to-charge values ordered from least intensity to greatest intensity. In certain embodiments, the bidirectional encoder 222 (e.g., BERT model) and the autoregressive decoder 224 (e.g., GPT model) may be further trained based on the sequence of tokens 228 (e.g., “(C)”, “n”, “c”, “2” “N”, “ . . . ”) ordered from least intensity to greatest intensity. For example, in one embodiment, a positional encoding of each token of the sequence of tokens 228 (e.g., “(C)”, “n”, “c”, “2” “N”, “ . . . ”) may be representative of an intensity of a mass-to-charge value (e.g., charged fragment) corresponding to a respective token. That is, in one embodiment, the positional layer of the of the bidirectional encoder 222 (e.g., BERT model) may be utilize to associate a respective intensity value or other contextual information with the sequence of tokens 228 (e.g., “(C)”, “n”, “c”, “2” “N”, “ . . . ”).

In another embodiment, the intensity values for each of the sequence of tokens 228 (e.g., “(C)”, “n”, “c”, “2” “N”, “ . . . ”) may be encoded utilizing the embedding layer of the bidirectional encoder 222 (e.g., BERT model). For example, the sequence of tokens 228 (e.g., “(C)”, “n”, “c”, “2” “N”, “ . . . ”) may be inputted into an embedding layer of the bidirectional encoder 222 (e.g., BERT model) to encode the sequence of tokens 228 (e.g., “(C)”, “n”, “c”, “2” “N”, “ . . . ” with contextual data) into a vector representation, and a subset of the vector representation may be modified to include an intensity value for each charged fragment corresponding to the sequence of tokens 228 (e.g., “(C)”, “n”, “c”, “2” “N”, “ . . . ”). In this way, the bidirectional encoder 222 (e.g., BERT model) may encode, for example, a proxy value for intensity, which may be utilized downstream as part of the prediction output generated by the autoregressive decoder 224 (e.g., GPT model).

### 6. Running Example of Inference Phase of a Pre-Trained and Fine-Tuned Bidirectional Transformer-Based Machine-Learning Model for Predicting Chemical Structures Utilizing Tokenizations of MS Data

FIG. 2G illustrates a running example 200G of the inference phase of a bidirectional transformer-based machine-learning model pre-trained and fine-tuned as discussed above with respect to FIGS. 2E and 2F, respectively. In certain embodiments, as depicted by FIG. 2G, during the inference phase of the trained bidirectional encoder 222 (e.g., BERT model) and the trained autoregressive decoder 224 (e.g., GPT model), the trained subword tokenizer 220 (e.g., BPE tokenizer, WordPiece tokenizer, Unigram tokenizer, BPE dropout tokenizer, and so forth) may receive MS input data 242 and generate a sequence of tokens 244 (e.g., “T1”, “T2”, “T3”, “T4” “T5”, “ . . . ”). In one embodiment, the sequence of tokens 244 (e.g., “T1”, “T2”, “T3”, “T4” “T5”, “ . . . ”) may represent one or more text strings or vector representations corresponding to, for example, mass spectral peaks derived from one or more unidentified molecules, compounds, or small molecules (e.g., metabolites). In certain embodiments, as further depicted by FIG. 2G, the trained subword tokenizer 220 may output the sequence of tokens 244 (e.g., “T1”, “T2”, “T3”, “T4” “T5”, “ . . . ”) into the trained randomly initialized encoder 233 (e.g., NLP model). It should be appreciated that the running example 200G may represent only one embodiment of the bidirectional transformer-based machine-learning model. For example, in other embodiments, the randomly initialized encoder 233 (e.g., NLP model) may not be included as part of bidirectional transformer-based machine-learning model architecture. Thus, in such embodiments, the trained subword tokenizer 220 may output the sequence of tokens 244 (e.g., “T1”, “T2”, “T3”, “T4” “T5”, “ . . . ”) directly to the bidirectional encoder 222 (e.g., BERT model).

In certain embodiments, the randomly initialized encoder 233 (e.g., NLP model) may then generate an output that may be received by the trained bidirectional encoder 222 (e.g., BERT model). The trained bidirectional encoder 222 (e.g., BERT model) and the trained autoregressive decoder 224 (e.g., GPT model) may then proceed as discussed above with respect to FIGS. 2E and 2F, respectively, to translate (e.g., machine translation) the sequence of tokens 244 (e.g., “T1”, “T2”, “T3”, “T4” “T5”, “ . . . ”) into a prediction of an output sequence of tokens 248 (e.g., “(C)”, “n”, “c”, “2” “N”, “ . . . ”) corresponding to a machine translation of the sequence of tokens 244 (e.g., “T1”, “T2”, “T3”, “T4” “T5”, “ . . . ”) representing a data set of mass spectral peaks derived from the MS input data 242 into a prediction of an output sequence of tokens 248 (e.g., “(C)”, “n”, “c”, “2” “N”, “ . . . ”) corresponding to one or more SMILES strings or other similar textual representations of compounds, molecules, or small molecules (e.g., metabolites).

### 7. Inference Phase of a Pre-Trained and Fine-Tuned Bidirectional Transformer-Based Machine-Learning Model for Predicting Chemical Structures Utilizing Sinusoidal Embeddings of MS Data

In certain embodiments, instead of predicting chemical structures or chemical properties based on tokenizations of the MS input data 242 (e.g., mass spectral peak m/z values), for example, it may be useful to provide techniques to predict chemical structures or chemical properties based on sinusoidal embeddings of the MS input data 242. Specifically, in some embodiments, the MS input data 242 may be measured at very high precision (e.g., 5 parts-per-million (ppm), 10 ppm, or greater). Thus, in some embodiments, relying on tokenizations of the MS input data 242 (e.g., mass spectral peak m/z values) alone may result in the MS input data 242 being represented less precisely than its measured values. Accordingly, as discussed in greater detail below with respect to FIGS. 2H and 2I, it may be useful to encode the MS input data 242, for example, as a sequence of sinusoidal embeddings (e.g., one or more vectors representing the m/z values of the MS input data 242 at a very high precision) before being inputted to the bidirectional transformer-based machine-learning model for predicting chemical structures and/or chemical properties of one or more compounds based thereon.

FIG. 2H illustrates a flow diagram 200H of a method for generating predictions of the chemical structure or chemical properties of molecules, compounds, and small molecules (e.g., metabolites) based on sinusoidal embeddings of MS data, in accordance with the presently disclosed embodiments. The flow diagram 200H may be performed utilizing one or more processing devices (e.g., computational metabolomics computing system 500) that may include hardware (e.g., a general purpose processor, a graphic processing unit (GPU), an application-specific integrated circuit (ASIC), a system-on-chip (SoC), a microcontroller, a field-programmable gate array (FPGA), a central processing unit (CPU), an application processor (AP), a visual processing unit (VPU), a neural processing unit (NPU), a neural decision processor (NDP), a deep learning processor (DLP), a tensor processing unit (TPU), or any other processing device(s) that may be suitable for processing genomics data, metabolomics data, proteomics data, metagenomics data, transcriptomics data, and/or various other omics data), software (e.g., instructions running/executing on one or more processors), firmware (e.g., microcode), or some combination thereof.

The flow diagram 200H may begin at block 250 with the one or more processing devices receiving MS data including a plurality of mass-to-charge values associated with fragments obtained from mass spectrometry performed on the compound. The flow diagram 200H may then continue at block 254 with the one or more processing devices generating a plurality of sinusoidal embeddings based on the plurality of mass-to-charge values. The flow diagram 200H may then continue at block 256 with the one or more processing devices inputting the plurality of sinusoidal embeddings into a bidirectional transformer-based machine-learning model trained to generate one or more predictions of a chemical structure of the compound based on the plurality of sinusoidal embeddings. The flow diagram 200H may then conclude at block 258 with the one or more processing devices outputting, by the bidirectional transformer-based machine-learning model, the one or more predictions of the chemical structure of the compound.

### 8. Running Example of Inference Phase of a Pre-Trained and Fine-Tuned Bidirectional Transformer-Based Machine-Learning Model for Predicting Chemical Structures Utilizing Sinusoidal Embeddings of MS Data

FIG. 2I illustrates a running example 200I of the inference phase of a bidirectional transformer-based machine-learning model pre-trained and fine-tuned to generate predictions of the chemical structure of a compound utilizing sinusoidal embeddings of MS data, in accordance with the presently disclosed embodiments. For example, in certain embodiments, as noted above, to better capture the very high precision of the MS input data 242 (e.g., mass spectral peak m/z values), the embedding layer may encode a sequence of fixed values or vectors 250 (e.g., “m/z₁”, “m/z₂”, “m/z₃”, “m/z₄” “m/z₅”, “ . . . ”), in which each m/z value may be represented by a d-dimensional vector corresponding to fixed values or vectors 258 (e.g., “m/z₁”, “m/z₂”, “m/z₃”, “m/z₄” “m/z₅”, “ . . . ”). For example, in one embodiment, the sinusoidal embeddings of the MS input data 242 (e.g., mass spectral peak m/z values) may be computed based on a sinusoidal function, which may be expressed as:

$$SE ( m / z , 2 i ; d ) = sin ( 2 {π [ {{λ}_{min} ( \frac{{λ}_{max}}{{λ}_{min}} )}^{2 i / ( d - 2 )} ]}^{- 1} m / z ) \\ SE ( m / z , 2 i + 1 ; d ) = cos ( 2 {π [ {{λ}_{min} ( \frac{{λ}_{max}}{{λ}_{min}} )}^{2 i / ( d - 2 )} ]}^{- 1} m / z ) . \qquad ( Equation 1 )$$

As may be appreciated by Equation 1, in some embodiments, the embeddings layer may include sinusoidal embeddings, which may interleave a sine curve and a cosine curve with sine values for even indexes and cosine values for odd indexes, or vice-versa. For example, referring again to Equation 1, m/z may represent the m/z values of the MS input data 242 (e.g., mass spectral peak m/z values), d may represent the length of the embedding vector, i may represent the index value into the embedding vector, and

$$2 π * {⌊ {λmin ( \frac{λmin}{λmax} )}^{2 i / ( d - 2 )} ⌋}^{- 1}$$

may represent the mass for element i of embedding vector length d. Similarly,

$${λmin ( \frac{λmin}{λmax} )}^{2 i / ( d - 2 )}$$

may represent a sequence of frequencies selected, such that the corresponding wavelengths across the embedding vector length d may be logarithmically distributed between λmin and λmax. For example, in one embodiment, λmin may include a value less than or equal to approximately 0.01. Likewise, in one embodiment, λmax may include a value greater than or equal to approximately 1,000.

Accordingly, the sinusoidal embeddings of the MS input data 242 (e.g., mass spectral peak m/z values) may enable learning representations of ultra-high resolution mass spectrometry data. Indeed, the sinusoidal embeddings, as set forth by Equation 1, may include sine and cosine values with wavelengths that are log-spaced across the range of sequences to be predicted by the bidirectional transformer-based machine-learning model as illustrated by the running example 200HI. In this way, the bidirectional transformer-based machine-learning model may better predict the chemical structure of a compound utilizing MS data and/or better predict the chemical properties of a compound utilizing MS data by reducing the number of predicted candidates due to including higher resolution sinusoidal embeddings.

For example, as further depicted by the running example 200I, in certain embodiments, the randomly initialized encoder 233 (e.g., NLP model) may receive the sequence of fixed values or vectors 258 (e.g., “m/z₁”, “m/z₂”, “m/z₃”, “m/z₄” “m/z₅”, “ . . . ”), and then generate an output that may be received by the trained bidirectional encoder 222 (e.g., BERT model). The trained bidirectional encoder 222 (e.g., BERT model) and the trained autoregressive decoder 224 (e.g., GPT model) may then proceed to translate (e.g., machine translation) the sequence of fixed values or vectors 258 (e.g., “m/z₁”, “m/z₂”, “m/z₃”, “m/z₄” “m/z₅”, “ . . . ”) into a prediction of an output sequence of tokens 248 (e.g., “(C)”, “n”, “c”, “2” “N”, “ . . . ”) corresponding to a machine translation of the sequence of fixed values or vectors 258 (e.g., “m/z₁”, “m/z₂”, “m/z₃”, “m/z₄” “m/z₅”, “ . . . ”) into a prediction of an output sequence of tokens 248 (e.g., “(C)”, “n”, “c”, “2” “N”, “ . . . ”) corresponding to one or more SMILES strings or other similar textual representations of compounds, molecules, or small molecules (e.g., metabolites). It should be appreciated that the running example 200I may represent only one embodiment of the bidirectional transformer-based machine-learning model. For example, in other embodiments, the randomly initialized encoder 233 (e.g., NLP model) may not be included as part of bidirectional transformer-based machine-learning model architecture. Thus, in such embodiments, the trained subword tokenizer 220 may output the sequence of tokens 258 (e.g., “T1”, “T2”, “T3”, “T4” “T5”, “ . . . ”) directly to the bidirectional encoder 222 (e.g., BERT model).

### 9. Pre-Training and/or Fine-Tuning a Bidirectional Transformer-Based Machine-Learning Model for Predicting Chemical Structures Utilizing Tokenizations of MS Data Including Precursor Mass

In certain embodiments, in addition to inputting the MS data 104 (e.g., representing mass spectra peaks) into the trained bidirectional transformer-based machine-learning model 102, the trained bidirectional transformer-based machine-learning model may also receive a precursor mass (e.g., precursor m/z). For example, in some embodiments, the precursor mass (e.g., precursor m/z) may represent the mass of, for example, an un-fragmented one or more naturally-occurring and/or non-naturally-occurring molecules, compounds, or small molecules (e.g., metabolites) corresponding to the MS data 104. In one embodiment, as described below with respect to FIGS. 2J-2L, including the input of the precursor mass (e.g., precursor m/z) to the trained bidirectional transformer-based machine-learning model may improve the ability of the bidirectional transformer-based machine-learning model to accurately predict the chemical structure of a compound (e.g., as compared to the mass spectra peak data of the MS data 104 alone).

FIG. 2J illustrates a flow diagram 200J of a method for pre-training and/or fine-tuning a bidirectional transformer-based machine-learning model to generate predictions of the chemical structure of a compound utilizing MS data including precursor mass, in accordance with the presently disclosed embodiments. The flow diagram 200J may be performed utilizing one or more processing devices (e.g., computational metabolomics computing system 500) that may include hardware (e.g., a general purpose processor, a graphic processing unit (GPU), an application-specific integrated circuit (ASIC), a system-on-chip (SoC), a microcontroller, a field-programmable gate array (FPGA), a central processing unit (CPU), an application processor (AP), a visual processing unit (VPU), a neural processing unit (NPU), a neural decision processor (NDP), a deep learning processor (DLP), a tensor processing unit (TPU), or any other processing device(s) that may be suitable for processing genomics data, metabolomics data, proteomics data, metagenomics data, transcriptomics data, and/or various other omics data), software (e.g., instructions running/executing on one or more processors), firmware (e.g., microcode), or some combination thereof.

The flow diagram 200J may begin at block 260 with the one or more processing devices receiving mass spectrometry (MS) data including a plurality of mass-to-charge values and precursor mass value associated with a compound. The flow diagram 200J may then continue at block 262 with the one or more processing devices generating a plurality of tokens based on the plurality of mass-to-charge values and the precursor mass value, the plurality of tokens including a set of one or more corrupted tokens and uncorrupted tokens, and the one or more corrupted tokens being predetermined to selectively correspond to the precursor mass value. The flow diagram 200J may then conclude at block 264 with the one or more processing devices inputting the plurality of tokens into the transformer-based machine-learning model to generate a prediction of the one or more corrupted tokens based on the uncorrupted tokens, in which the prediction of the one or more corrupted tokens corresponding to an original sequence of tokens representative of the plurality of mass-to-charge values and the precursor mass value.

### 10. Running Examples of Pre-Training and/or Fine-Tuning a Bidirectional Transformer-Based Machine-Learning Model for Predicting Chemical Structures Utilizing Tokenizations of MS Data Including Precursor Mass

FIG. 2K illustrates one or more running example 200K for pre-training and/or fine-tuning a bidirectional transformer-based machine-learning model to generate predictions of the chemical structure of a compound, in accordance with the presently disclosed embodiments. In certain embodiments, as depicted by FIG. 2K, during the pre-training and/or fine-tuning phase of the bidirectional encoder 222 (e.g., BERT model) and the autoregressive decoder 224 (e.g., GPT model), the trained subword tokenizer 220 (e.g., BPE tokenizer, WordPiece tokenizer, Unigram tokenizer, BPE dropout tokenizer, and so forth) may receive MS training data 268. In certain embodiments, MS training data 268 may include a data set of mass spectra peak values and one or more precursor mass values, which may represent the mass of, for example, an un-fragmented one or more naturally-occurring and/or non-naturally-occurring molecules, compounds, or small molecules (e.g., metabolites).

In certain embodiments, the trained subword tokenizer 220 (e.g., BPE tokenizer. WordPiece tokenizer. Unigram tokenizer. BPE dropout tokenizer, and so forth) may then generate a sequence of tokens 236 (e.g., “T1”, “T2”, “P_{M}”, “T4” “T5”, “ . . . ”) based on the received MS training data 268. In one embodiment, the sequence of tokens 270 (e.g., “T1”, “T2”, “P_{M}”, “T4” “T5”, “ . . . ”) may represent one or more text strings or vector representations corresponding to, for example, a data set of mass spectral peaks and precursor mass derived from the MS training data 268. In certain embodiments, as will be further illustrated below by FIG. 2L, the token 272A (e.g., “P_{M}”) corresponding to the precursor mass (e.g., precursor m/z) may be selectively corrupted or masked by the trained subword tokenizer 220, such that the bidirectional transformer-based machine-learning model (e.g., the bidirectional encoder 222 and the autoregressive decoder 224) may be trained on the token 272A (e.g., “P_{M}”) corresponding to the precursor mass (e.g., precursor m/z) without potentially overfitting the bidirectional transformer-based machine-learning model (e.g., the bidirectional encoder 222 and the autoregressive decoder 224) to learn only, or be overly biased, to the precursor mass (e.g., precursor m/z).

For example, in one embodiment, the trained subword tokenizer 220 may selectively corrupt or mask the token 272A (e.g., “P_{M}”) corresponding to the precursor mass (e.g., precursor m/z), for example, 10% of the time, 15% of the time, 20% of the time, 25% of the time, 30% of the time, 35% of the time, 40% of the time, 45% of the time, 50% of the time, or may otherwise be determined heuristically through iterative tuning of the bidirectional transformer-based machine-learning model (e.g., the bidirectional encoder 222 and the autoregressive decoder 224). In one embodiment, the token 272A (e.g., “P_{M}”) may be corrupted, for example, utilizing any of various token corrupting processes, such as a token deletion process, a token masking process, a text infilling process, a text string permutation process, or a sequence rotation process. FIG. 2K illustrates an iteration of tuning of the bidirectional transformer-based machine-learning model (e.g., the bidirectional encoder 222 and the autoregressive decoder 224) in which the token 272A (e.g., “P_{M}”) corresponding to the precursor mass (e.g., precursor m/z) is inputted to the bidirectional transformer-based machine-learning model uncorrupted and/or unmasked. In contrast, as will be further illustrated below, FIG. 2L illustrates an iteration of tuning of the bidirectional transformer-based machine-learning model (e.g., the bidirectional encoder 222 and the autoregressive decoder 224) in which the token 272B (e.g., “_”) corresponding to the precursor mass (e.g., precursor m/z) is inputted to the bidirectional transformer-based machine-learning model corrupted and/or masked.

In certain embodiments, as further depicted by FIG. 2K, the trained subword tokenizer 220 may output the sequence of tokens 270 (e.g., “T1”, “T2”, “P_{M}”, “T4” “T5”, “ . . . ”) into a randomly initialized encoder 233 (e.g., NLP model) that may be suitable for learning contextual data (e.g., positional encodings and embeddings) based on the sequence of tokens 270 (e.g., “T1”, “T2”, “P_{M}”, “T4” “T5”, “ . . . ”). It should be appreciated that the running example 200K may represent only one embodiment of the bidirectional transformer-based machine-learning model. For example, in other embodiments, the randomly initialized encoder 233 (e.g., NLP model) may not be included as part of bidirectional transformer-based machine-learning model architecture. Thus, in such embodiments, the trained subword tokenizer 220 may output the sequence of tokens 270 (e.g., “T1”, “T2”, “P_{M}”, “T4” “T5”, “ . . . ”) directly to the bidirectional encoder 222 (e.g., BERT model).

In certain embodiments, the bidirectional encoder 222 (e.g., BERT model), the autoregressive decoder 224 (e.g., GPT model), and the randomly initialized encoder 233 may each be associated with a vocabulary 235. In certain embodiments, the vocabulary 235 may include any library including various individual characters, words, subwords, sequences of numerical values, sequences of sequential characters, sequences of sequential numerical values, and so forth that may be augmented and updated over time. In some embodiments, the vocabulary 235 may be accessed by the bidirectional encoder 222 (e.g., BERT model), the autoregressive decoder 224 (e.g., GPT model), and the randomly initialized encoder 233 during the pre-training phase and/or fine-tuning phase. In another embodiment, each of the bidirectional encoder 222 (e.g., BERT model), the autoregressive decoder 224 (e.g., GPT model), and the randomly initialized encoder 233 may be associated with its own vocabulary 235.

In certain embodiments, the randomly initialized encoder 233 (e.g., NLP model) may then generate an output that may be received by the bidirectional encoder 222 (e.g., BERT model). The bidirectional encoder 222 (e.g., BERT model) and the autoregressive decoder 224 (e.g., GPT model), utilizing the sequence of tokens 274 (e.g., “<S>”, “(C)”, “n”, “c”, “2”), may then translate (e.g., machine translation) the sequence of tokens 270 (e.g., “T1”, “T2”, “P_{M}”, “T4” “T5”, “ . . . ”) into a prediction of an output sequence of tokens 272 (e.g., “(C)”, “n”, “c”, “2” “N”, “ . . . ”) corresponding to a machine translation of the sequence of tokens 270 (e.g., “T1”, “T2”, “T3”, “T4” “T5”, “ . . . ”) representing a data set of mass spectral peaks and precursor mass derived from the MS training data 268 into a prediction of an output sequence of tokens 272 (e.g., “(C)”, “n”, “c”, “2” “N”, “ . . . ”) corresponding to one or more SMILES strings or other similar textual representations of compounds, molecules, or small molecules (e.g., metabolites).

### 11. Inference Phase of a Subword Tokenizer to be Utilized with a Bidirectional Transformer-Based Machine-Learning Model for Predicting Chemical Structures

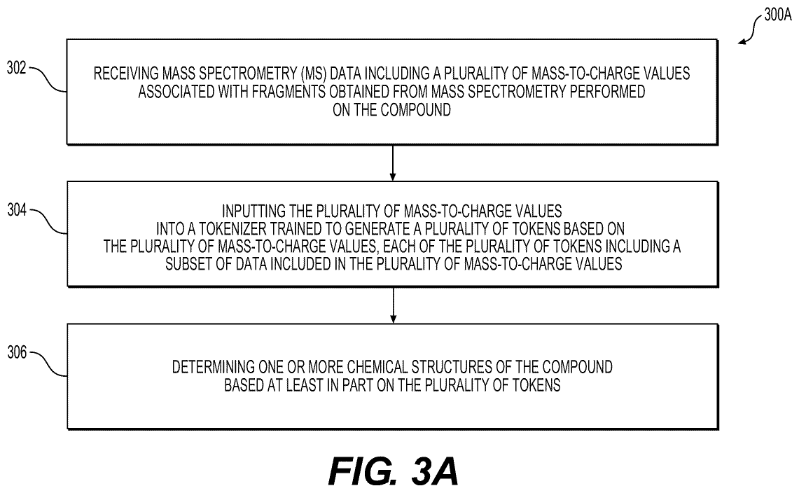

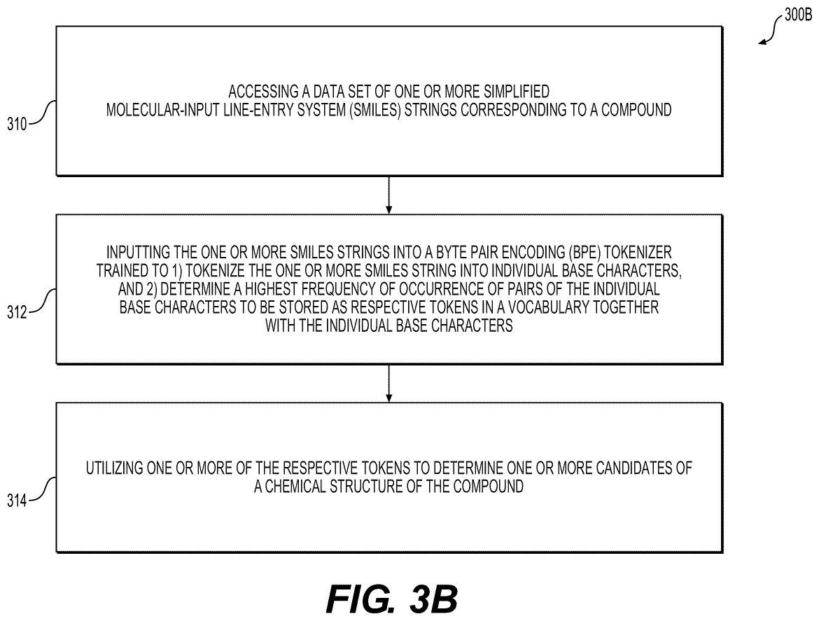

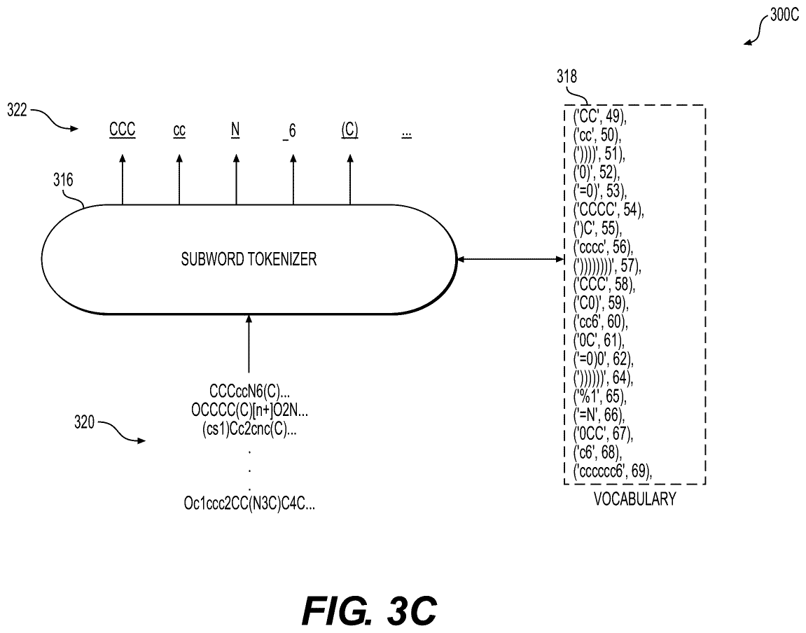

FIG. 3A illustrates a flow diagram 300A of a method for providing a subword tokenizer to be utilized with a bidirectional transformer-based machine-learning model to generate predictions of the chemical structure of a compound, in accordance with the presently disclosed embodiments. The flow diagram 300A may be performed utilizing one or more processing devices (e.g., computational metabolomics computing system 500) that may include hardware (e.g., a general purpose processor, a graphic processing unit (GPU), an application-specific integrated circuit (ASIC), a system-on-chip (SoC), a microcontroller, a field-programmable gate array (FPGA), a central processing unit (CPU), an application processor (AP), a visual processing unit (VPU), a neural processing unit (NPU), a neural decision processor (NDP), a deep learning processor (DLP), a tensor processing unit (TPU), or any other processing device(s) that may be suitable for processing genomics data, metabolomics data, proteomics data, metagenomics data, transcriptomics data, and/or various other omics data), software (e.g., instructions running/executing on one or more processors), firmware (e.g., microcode), or some combination thereof.

The flow diagram 300A may begin at block 302 with the one or more processing devices receiving MS data including a plurality of mass-to-charge values associated with fragments obtained from mass spectrometry performed on the compound. The flow diagram 300A may then continue at block 304 with the one or more processing devices inputting the plurality of mass-to-charge values into a tokenizer trained to generate a plurality of tokens based on the plurality of mass-to-charge values, each of the plurality of tokens including a subset of data included in the plurality of mass-to-charge values. The flow diagram 300A may then conclude at block 308 with the one or more processing devices determining one or more chemical structures of the compound based at least in part on the plurality of tokens.

### 12. Training Phase of a Subword Tokenizer to be Utilized with a Bidirectional Transformer-Based Machine-Learning Model for Predicting Chemical Structures

FIG. 3B illustrates a flow diagram 300B of a method for training a subword tokenizer to be utilized with a bidirectional transformer-based machine-learning model to generate predictions of the chemical structure of a compound, in accordance with the presently disclosed embodiments. The flow diagram 300B may be performed utilizing one or more processing devices (e.g., computational metabolomics computing system 500) that may include hardware (e.g., a general purpose processor, a graphic processing unit (GPU), an application-specific integrated circuit (ASIC), a system-on-chip (SoC), a microcontroller, a field-programmable gate array (FPGA), a central processing unit (CPU), an application processor (AP), a visual processing unit (VPU), a neural processing unit (NPU), a neural decision processor (NDP), a deep learning processor (DLP), a tensor processing unit (TPU), or any other processing device(s) that may be suitable for processing genomics data, metabolomics data, proteomics data, metagenomics data, transcriptomics data, and/or various other omics data), software (e.g., instructions running/executing on one or more processors), firmware (e.g., microcode), or some combination thereof.

The flow diagram 300B may begin at block 310 with the one or more processing devices accessing a data set of one or more SMILES strings corresponding to a compound. The flow diagram 300B may then continue at block 312 with the one or more processing devices inputting the one or more SMILES strings into a byte pair encoding (BPE) tokenizer trained to 1) tokenize the one or more SMILES string into individual base characters, and 2) determine a highest frequency of occurrence of pairs of the individual base characters to be stored as respective tokens in a vocabulary together with the individual base characters. The flow diagram 300B may then conclude at block 314 with the one or more processing devices utilizing one or more of the respective tokens to determine one or more candidates of a chemical structure of the compound. It should appreciated that while FIG. 3B is illustrated with respect to training a BPE subword tokenizer, in some embodiments, one or more steps of the flow diagram 300B may be suitable for training, for example, one or more WordPiece subword tokenizers, Unigram subword tokenizers, BPE dropout subword tokenizers, and so forth.

FIG. 3C illustrates an example embodiment of a workflow diagram 300C for training a subword tokenizer 316 (and associated vocabulary 318) to be utilized with a bidirectional transformer-based machine-learning model to generate predictions of the chemical structure of a compound, in accordance with the presently disclosed embodiments. In certain embodiments, as depicted by FIG. 3C, during the training phase, the subword tokenizer 316 (e.g., BPE tokenizer, WordPiece tokenizer, Unigram tokenizer, BPE dropout tokenizer, and so forth) may receive one or more textual strings 320. In certain embodiments, the one or more textual strings 320 may include, for example, one or more SMILES strings, DeepSMILES strings, SELFIES strings, or other similar textual representations of compounds, molecules, or small molecule (e.g., metabolites). For example, in some embodiments, the subword tokenizer 320 may be trained by iteratively providing large data sets of textual strings 320 (e.g., SMILES strings “CCCcc0N6(C) . . . ”, “OCCCC(C)[n+]O2N . . . ”, “(cs1)Cc2cnc(C) . . . ”, “ . . . ”, and “Oc1ccc2CC(N3C)C4C . . . ”) to the subword tokenizer 316 to learn individual base characters (e.g., “(C)” “C”, “O”, “2”, “4”, “c”, “n”, “0”, and so forth) and frequently occurring sequential characters (e.g., “CCC”, “nc”, “CC”, and so forth).

In certain embodiments, the subword tokenizer 316 may then tokenize the one or more textual strings 320 (e.g., SMILES strings “CCCcc0N6(C) . . . ”, “OCCCC(C)[n+]O2N . . . ”, “(cs1)Cc2cnc(C) . . . ”, “ . . . ”, and “Oc1ccc2CC(N3C)C4C . . . ”) into one or more sequences of tokens 322 (e.g., “CCC”, “cc”, “0”, “N”, “(C)”, “ . . . ”, and so forth) and store the one or more sequences of tokens 322 (e.g., “CCC”, “cc”, “0”, “N”, “(C)”, “ . . . ”, and so forth) into the vocabulary 318 associated with the subword tokenizer 316 to be utilized in future tokenizations performed by the trained subword tokenizer 316. Specifically, the subword tokenizer 316 may learn the individual base characters (e.g., “(C)” “C”, “O”, “2”, “4”, “c”, “n”, “0”, and so forth) and the frequently occurring sequential characters (e.g., “CCC”, “nc”, “CC”, and so forth), and then store the individual base characters (e.g., “(C)” “C”, “O”, “2”, “4”, “c”, “n”, “0”, and so forth) together with the frequently occurring sequential characters (e.g., “CCC”, “nc”, “CC”, and so forth) in the vocabulary 318 as characters and subwords, respectively.

For example, in certain embodiments, the vocabulary 318 may include any library including various individual characters, words, subwords, sequences of numerical values, sequences of sequential characters, sequences of sequential numerical values, and so forth that may be augmented and updated over time based on patterns learned by the subword tokenizer 316. This may thus allow the subword tokenizer 316 to become adept at tokenizing SMILES strings, which may be utilized to train one or more bidirectional transformer-based machine-learning models to infer SMILES strings from inputted mass spectra, in accordance with the presently disclosed embodiments.

### 13. Inference and Training Phases of a Trained Bidirectional Transformer-Based Machine-Learning Model for Predicting Chemical Structures

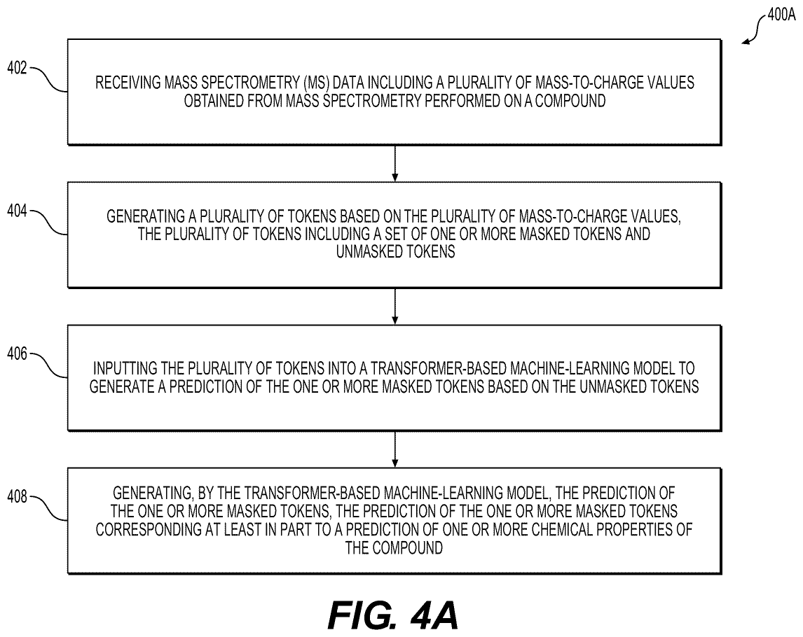

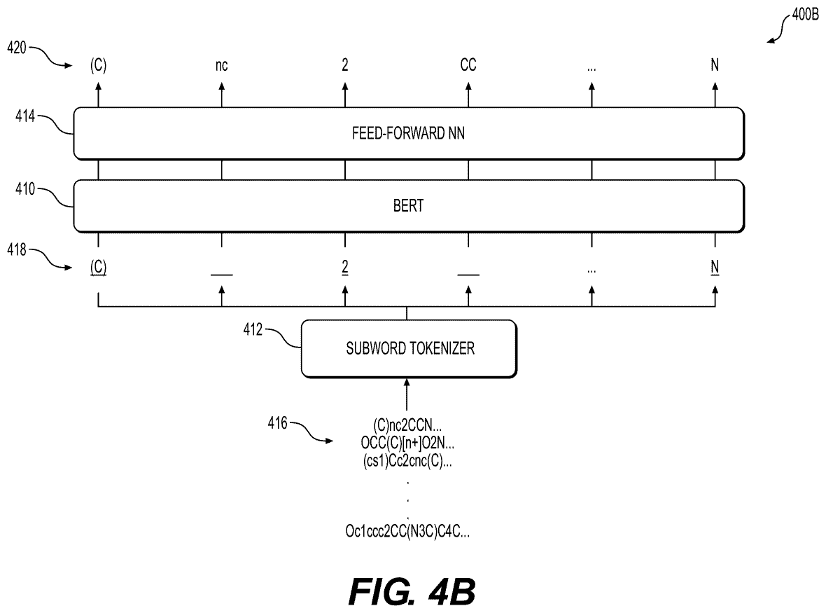

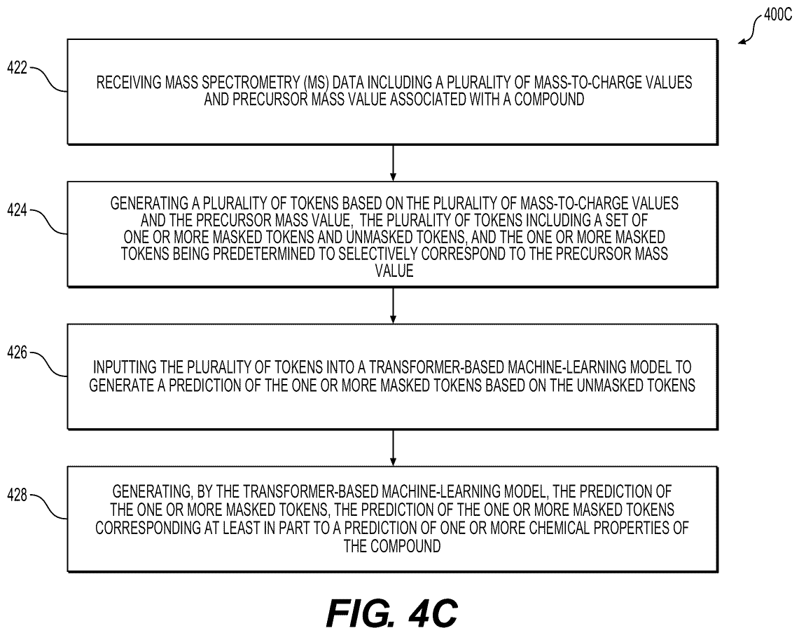

FIG. 4A illustrates a flow diagram 400A of a method for generating predictions of one or more chemical properties of a compound based on MS data, in accordance with the presently disclosed embodiments. The flow diagram 400A may be performed utilizing one or more processing devices (e.g., computational metabolomics computing system 500) that may include hardware (e.g., a general purpose processor, a graphic processing unit (GPU), an application-specific integrated circuit (ASIC), a system-on-chip (SoC), a microcontroller, a field-programmable gate array (FPGA), a central processing unit (CPU), an application processor (AP), a visual processing unit (VPU), a neural processing unit (NPU), a neural decision processor (NDP), a deep learning processor (DLP), a tensor processing unit (TPU), or any other processing device(s) that may be suitable for processing genomics data, metabolomics data, proteomics data, metagenomics data, transcriptomics data, and/or various other omics data), software (e.g., instructions running/executing on one or more processors), firmware (e.g., microcode), or some combination thereof.

The flow diagram 400A may begin at block 402 with the one or more processing devices receiving MS data including a plurality of mass-to-charge values obtained from mass spectrometry performed on a compound. The flow diagram 400A may then continue at block 404 with the one or more processing devices generating a plurality of tokens based on the plurality of mass-to-charge values, the plurality of tokens including a set of one or more masked tokens and unmasked tokens. The flow diagram 400A may then continue at block 406 with the one or more processing devices inputting the plurality of tokens into a transformer-based machine-learning model to generate a prediction of the one or more masked tokens based on the unmasked tokens. The flow diagram 400A may then conclude at block 408 with the one or more processing devices generating, by the transformer-based machine-learning model, the prediction of the one or more masked tokens, the prediction of the one or more masked tokens corresponding at least in part to a prediction of one or more chemical properties of the compound.

FIG. 4B illustrates a running example 400B for generating predictions of one or more chemical properties of a compound based on MS data utilizing a BERT model 410, in accordance with the presently disclosed embodiments. In certain embodiments, as depicted by FIG. 4B, the trained subword tokenizer 412 (e.g., BPE tokenizer, WordPiece tokenizer, Unigram tokenizer, BPE dropout tokenizer, and so forth) may receive one or more textual strings 416 (e.g., SMILES strings “(C)nc2CCN . . . ”, “OCC(C)[n+]O2N . . . ”, “(cs1)Cc2cnc(C) . . . ”, “ . . . ”, and “Oc1ccc2CC(N3C)C4C . . . ”) and generate a sequence of tokens 418 (e.g., “C”, “_”, “2”, “ . . . ”, and “N”) by tokenizing (e.g., based on the vocabulary 318) the one or more textual strings 416. In certain embodiments, trained subword tokenizer 412 (e.g., BPE tokenizer, WordPiece tokenizer, Unigram tokenizer, BPE dropout tokenizer, and so forth) may include a subword tokenizer trained in accordance the techniques discussed above with respect to FIGS. 3B and 3C. In certain embodiments, as depicted, one or more tokens of the sequence of tokens 418 (e.g., “C”, “_”, “2”, “_”, “ . . . ” and “N”) may be masked, and the BERT model 410 may be trained to predict the one or more masked tokens (e.g., “_”, “_”,) of the sequence of tokens 418 (e.g., “C”, “_”, “2”, “_”, “ . . . ”, and “N”) based on the one or more unmasked tokens (e.g., “C”, “2”, “ . . . ”, and “N”) of the sequence of tokens 418 (e.g., “C”, “_”, “2”, “_”, “ . . . ”, and “N”). Indeed, in certain embodiments, the BERT model 410 may be iteratively trained utilizing, for example, one or more mask language modeling (MLM) processes and/or one or more next-sentence prediction (NSP) processes to learn the grammar, context, and syntax of SMILES stings, DeepSMILES strings, or SELFIES strings to be learned to predict chemical properties of one or more scientifically unidentified molecules, compounds, or small molecules (e.g., metabolites).

In certain embodiments, the BERT model 410 may generate an output to a feedforward neural network (NN) 414 that may be utilize to generate an output sequence of tokens 420 (e.g., “(C)”, “nc”, “2”, “CC”, “ . . . ”, “N”) corresponding to the original unmasked sequence of tokens (e.g., “C”, “nc”, “2”, “CC”, “ . . . ”, and “N”). In some embodiments, after the BERT model 410 is sufficiently trained, the BERT model 410 may be then utilized to generate predictions of chemical properties of molecules, compounds, and small molecules (e.g., metabolites) based on MS data in accordance with the presently disclosed embodiments. For example, in one embodiment, the output sequence of tokens 420 (e.g., “(C)”, “nc”, “2”, “CC”, “ . . . ”, “N”) prediction may include one or more SMILES strings representative of one or more predicted chemical properties.

FIG. 4C illustrates a flow diagram 400C of a method for generating predictions of one or more chemical properties of a compound based on MS data including precursor mass, in accordance with the presently disclosed embodiments. The flow diagram 400C may be performed utilizing one or more processing devices (e.g., computational metabolomics computing system 500) that may include hardware (e.g., a general purpose processor, a graphic processing unit (GPU), an application-specific integrated circuit (ASIC), a system-on-chip (SoC), a microcontroller, a field-programmable gate array (FPGA), a central processing unit (CPU), an application processor (AP), a visual processing unit (VPU), a neural processing unit (NPU), a neural decision processor (NDP), a deep learning processor (DLP), a tensor processing unit (TPU), or any other processing device(s) that may be suitable for processing genomics data, metabolomics data, proteomics data, metagenomics data, transcriptomics data, and/or various other omics data), software (e.g., instructions running/executing on one or more processors), firmware (e.g., microcode), or some combination thereof.

In certain embodiments, the flow diagram 400C may proceed similarly as discussed above with respect to the flow diagram 400A and with respect to the running example 400B, with the exception that the flow diagram 400C may include generating predictions of one or more chemical properties of a compound based on MS data including both mass spectra peaks and precursor mass. For example, the flow diagram 400C may begin at block 422 with the one or more processing devices receiving MS data including a plurality of mass-to-charge values and a precursor mass value associated with a compound. The flow diagram 400C may then continue at block 424 with the one or more processing devices generating a plurality of tokens based on the plurality of mass-to-charge values and the precursor mass value, the plurality of tokens including a set of one or more masked tokens and unmasked tokens, and the one or more masked tokens being predetermined to selectively correspond to the precursor mass value. The flow diagram 400C may then continue at block 426 with the one or more processing devices inputting the plurality of tokens into a transformer-based machine-learning model to generate a prediction of the one or more masked tokens based on the unmasked tokens. The flow diagram 400C may then conclude at block 428 with the one or more processing devices generating, by the transformer-based machine-learning model, the prediction of the one or more masked tokens, the prediction of the one or more masked tokens corresponding at least in part to a prediction of one or more chemical properties of the compound.

### 14. Generating Training Data for a Bidirectional Transformer-Based Machine-Learning Model for Predicting Chemical Structures

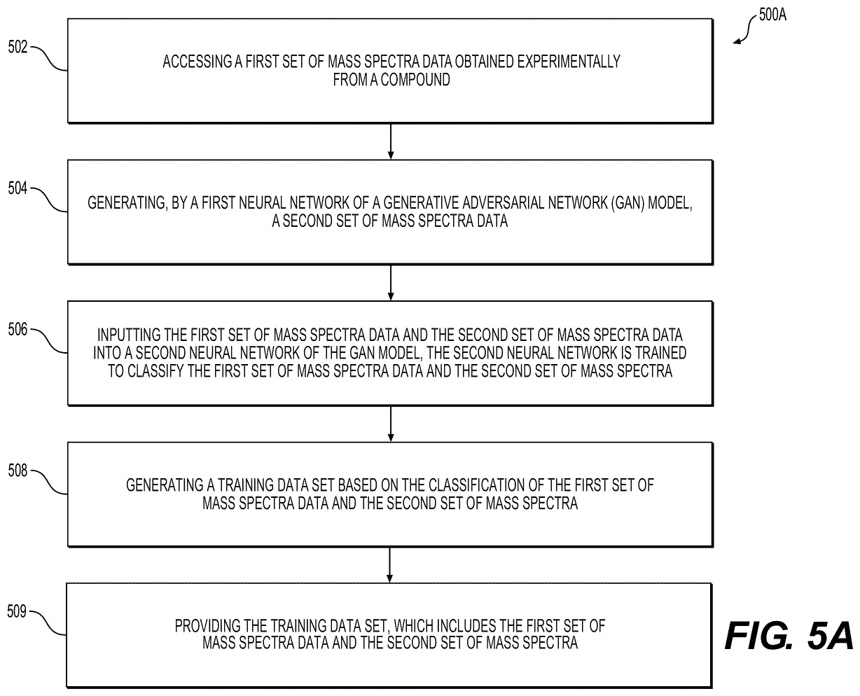

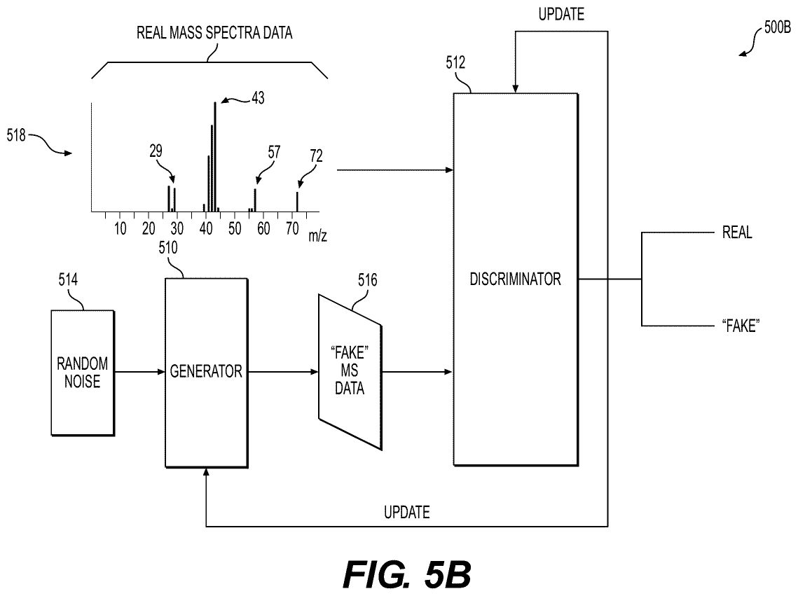

FIG. 5A illustrates a flow diagram 500A of a method for generating training data for a bidirectional transformer-based machine-learning model trained to generate predictions of the chemical structure of a compound based on MS data, in accordance with the presently disclosed embodiments. The flow diagram 500A may be performed utilizing one or more processing devices (e.g., computational metabolomics computing system 600) that may include hardware (e.g., a general purpose processor, a graphic processing unit (GPU), an application-specific integrated circuit (ASIC), a system-on-chip (SoC), a microcontroller, a field-programmable gate array (FPGA), a central processing unit (CPU), an application processor (AP), a visual processing unit (VPU), a neural processing unit (NPU), a neural decision processor (NDP), a deep learning processor (DLP), a tensor processing unit (TPU), or any other processing device(s) that may be suitable for processing genomics data, metabolomics data, proteomics data, metagenomics data, transcriptomics data, and/or various other omics data), software (e.g., instructions running/executing on one or more processors), firmware (e.g., microcode), or some combination thereof.

The flow diagram 500A may begin at block 502 with the one or more processing devices accessing a first set of mass spectra data obtained experimentally from a compound. The flow diagram 500A may then continue at block 504 with the one or more processing devices generating, by a first neural network of a generative adversarial network (GAN) model, a second set of mass spectra data. The flow diagram 500A may then continue at block 506 with the one or more processing devices inputting the first set of mass spectra data and the second set of mass spectra data into a second neural network of the GAN model, the second neural network is trained to classify the first set of mass spectra data and the second set of mass spectra. The flow diagram 500A may then continue at block 508 with the one or more processing devices generating a training data set based on the classification of the first set of mass spectra data and the second set of mass spectra. The flow diagram 500A may then conclude at block 509 with the one or more processing devices providing the training data set, which includes the first set of mass spectra data and the second set of mass spectra data.

FIG. 5B illustrate a running example 500B for generating training data for a bidirectional transformer-based machine-learning model trained to generate predictions of the chemical structure of a compound based on MS data, in accordance with the presently disclosed embodiments. In certain embodiments, the running example 500B may be illustrated with respect to a generative adversarial network (GAN), which may include a generator model 510 (e.g., a first neural network (NN)) and discriminator model 512 (e.g., a second neural network (NN)) that may be trained and executed concurrently. In one embodiment, based on random noise data 514, the generator model 510 (e.g., a first neural network (NN)) may generate “fake” MS data 516. For example, in one embodiment, the “fake” MS data 516 may include synthetic data, or otherwise MS data corresponding to one or more non-naturally-occurring molecules, compounds, or small molecules (e.g., metabolites). In another embodiment, based on random noise data 514 and at least partially on “real” MS data 518, the generator model 510 (e.g., a first neural network (NN)) may generate “fake” MS data 516.

In certain embodiments, the discriminator model 512 (e.g., a second neural network (NN)) may access “real” MS data 518, which may include MS data obtained experimentally from a compound. For example, in one embodiment, the “real” MS data 518 may include MS data corresponding to one or more naturally-occurring molecules, compounds, or small molecules (e.g., metabolites). In certain embodiments, the discriminator model 512 (e.g., a second neural network (NN)) may receive the “fake” MS data 516 and the “real” MS data 518 and attempt to classify the “fake” MS data 516 and the “real” MS data 518 as being “Real” or “Fake”. In accordance with the presently disclosed embodiments, the generator model 510 (e.g., a first neural network (NN)) and the discriminator model 512 (e.g., a second neural network (NN)) may be iteratively updated until the discriminator model 512 (e.g., a second neural network (NN)) is no longer correctly classifying the “fake” MS data 516 as being “Fake”, and is instead classifying the “fake” MS data 516 as being “Real” (e.g., thus indicating that predictions from any machine-learning model to be trained based on the “fake” MS data 516 can be “trusted” and relied upon because the “fake” MS data 516 is being interpreted by the model as being indistinguishable from the “real” MS data 518).

In certain embodiments, the “fake” MS data 516 may be then stored together with the “real” MS data 518 as training data, and may be utilized to train, for example, one or more bidirectional transformer-based machine-learning models to predict the chemical structure or chemical properties of molecules, compounds, or small molecules (e.g., metabolites), particularly in the case in which “real” MS data 518 is available in insufficient quantity to accurately train the one or more bidirectional transformer-based machine-learning models. Thus, by training and utilizing the generator model 510 (e.g., a first neural network (NN)) and the discriminator model 512 (e.g., a second neural network (NN)) to generate and infer the “fake” MS data 516, large training data sets for training the one or more bidirectional transformer-based machine-learning models may be produced. In this way, the training data sets based on the “fake” MS data 516 and the “real” MS data 518 may include MS data for molecules or compounds having a wide array of diversity, as oppose to training data sets based on only the “real” MS data 518 (e.g., which may have limited availability since it can come from only naturally-occurring chemical or biochemical samples and that which exist at a reasonable level of purity).

### 15. Computing and Artificial Intelligence (AI) Systems Suitable for Predicting Chemical Structures and Chemical Properties

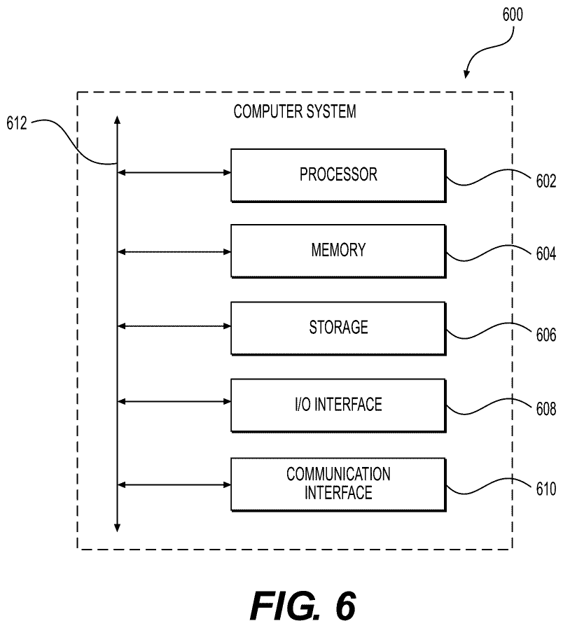

FIG. 6 illustrates an example computational metabolomics computing system 600 that may be utilized to generate predictions of the chemical structure or chemical properties of molecules, compounds, and small molecules (e.g., metabolites) based on MS data, in accordance with the presently disclosed embodiments. In certain embodiments, one or more computational metabolomics computing systems 600 perform one or more steps of one or more methods described or illustrated herein. In certain embodiments, one or more computational metabolomics computing system 600 provide functionality described or illustrated herein. In certain embodiments, software running on one or more computational metabolomics computing system 600 performs one or more steps of one or more methods described or illustrated herein or provides functionality described or illustrated herein. Certain embodiments include one or more portions of one or more computational metabolomics computing systems 600. Herein, reference to a computer system may encompass a computing device, and vice versa, where appropriate. Moreover, reference to a computer system may encompass one or more computer systems, where appropriate.

This disclosure contemplates any suitable number of computational metabolomics computing systems 600. This disclosure contemplates computational metabolomics computing system 600 taking any suitable physical form. As example and not by way of limitation, computational metabolomics computing system 600 may be an embedded computer system, a system-on-chip (SOC), a single-board computer system (SBC) (e.g., a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, a tablet computer system, an augmented/virtual reality device, or a combination of two or more of these. Where appropriate, computational metabolomics computing system 600 may include one or more computational metabolomics computing systems 600; be unitary or distributed; span multiple locations; span multiple machines; span multiple data centers; or reside in a cloud, which may include one or more cloud components in one or more networks.

Where appropriate, one or more computational metabolomics computing system 600 may perform without substantial spatial or temporal limitation one or more steps of one or more methods described or illustrated herein. As an example, and not by way of limitation, one or more computational metabolomics computing system 600 may perform in real time or in batch mode one or more steps of one or more methods described or illustrated herein. One or more computational metabolomics computing system 600 may perform at different times or at different locations one or more steps of one or more methods described or illustrated herein, where appropriate.

In certain embodiments, computational metabolomics computing system 600 includes a processor 602, memory 604, storage 606, an input/output (I/O) interface 608, a communication interface 610, and a bus 512. Although this disclosure describes and illustrates a particular computer system having a particular number of particular components in a particular arrangement, this disclosure contemplates any suitable computer system having any suitable number of any suitable components in any suitable arrangement. In certain embodiments, processor 602 includes hardware for executing instructions, such as those making up a computer program. As an example, and not by way of limitation, to execute instructions, processor 602 may retrieve (or fetch) the instructions from an internal register, an internal cache, memory 604, or storage 606; decode and execute them; and then write one or more results to an internal register, an internal cache, memory 604, or storage 606. In certain embodiments, processor 602 may include one or more internal caches for data, instructions, or addresses. This disclosure contemplates processor 602 including any suitable number of any suitable internal caches, where appropriate. As an example, and not by way of limitation, processor 602 may include one or more instruction caches, one or more data caches, and one or more translation lookaside buffers (TLBs). Instructions in the instruction caches may be copies of instructions in memory 604 or storage 606, and the instruction caches may speed up retrieval of those instructions by processor 602.

Data in the data caches may be copies of data in memory 604 or storage 606 for instructions executing at processor 602 to operate on; the results of previous instructions executed at processor 602 for access by subsequent instructions executing at processor 602 or for writing to memory 604 or storage 606; or other suitable data. The data caches may speed up read or write operations by processor 602. The TLBs may speed up virtual-address translation for processor 602. In certain embodiments, processor 602 may include one or more internal registers for data, instructions, or addresses. This disclosure contemplates processor 602 including any suitable number of any suitable internal registers, where appropriate. Where appropriate, processor 602 may include one or more arithmetic logic units (ALUs); be a multi-core processor; or include one or more processors 602. Although this disclosure describes and illustrates a particular processor, this disclosure contemplates any suitable processor.

In certain embodiments, memory 604 includes main memory for storing instructions for processor 602 to execute or data for processor 602 to operate on. As an example, and not by way of limitation, computational metabolomics computing system 600 may load instructions from storage 606 or another source (such as, for example, another computational metabolomics computing system 600) to memory 604. Processor 602 may then load the instructions from memory 604 to an internal register or internal cache. To execute the instructions, processor 602 may retrieve the instructions from the internal register or internal cache and decode them. During or after execution of the instructions, processor 602 may write one or more results (which may be intermediate or final results) to the internal register or internal cache. Processor 602 may then write one or more of those results to memory 604. In certain embodiments, processor 602 executes only instructions in one or more internal registers or internal caches or in memory 604 (as opposed to storage 606 or elsewhere) and operates only on data in one or more internal registers or internal caches or in memory 604 (as opposed to storage 606 or elsewhere).

One or more memory buses (which may each include an address bus and a data bus) may couple processor 602 to memory 604. Bus 512 may include one or more memory buses, as described below. In certain embodiments, one or more memory management units (MMUs) reside between processor 602 and memory 604 and facilitate accesses to memory 604 requested by processor 602. In certain embodiments, memory 604 includes random access memory (RAM). This RAM may be volatile memory, where appropriate. Where appropriate, this RAM may be dynamic RAM (DRAM) or static RAM (SRAM). Moreover, where appropriate, this RAM may be single-ported or multi-ported RAM. This disclosure contemplates any suitable RAM. Memory 604 may include one or more memory devices 604, where appropriate. Although this disclosure describes and illustrates particular memory, this disclosure contemplates any suitable memory.

In certain embodiments, storage 606 includes mass storage for data or instructions. As an example, and not by way of limitation, storage 606 may include a hard disk drive (HDD), a floppy disk drive, flash memory, an optical disc, a magneto-optical disc, magnetic tape, or a Universal Serial Bus (USB) drive or a combination of two or more of these. Storage 606 may include removable or non-removable (or fixed) media, where appropriate. Storage 606 may be internal or external to computational metabolomics computing system 600, where appropriate. In certain embodiments, storage 606 is non-volatile, solid-state memory. In certain embodiments, storage 606 includes read-only memory (ROM). Where appropriate, this ROM may be mask-programmed ROM, programmable ROM (PROM), erasable PROM (EPROM), electrically erasable PROM (EEPROM), electrically alterable ROM (EAROM), or flash memory or a combination of two or more of these. This disclosure contemplates mass storage 606 taking any suitable physical form. Storage 606 may include one or more storage control units facilitating communication between processor 602 and storage 606, where appropriate. Where appropriate, storage 606 may include one or more storages 606. Although this disclosure describes and illustrates particular storage, this disclosure contemplates any suitable storage.

In certain embodiments, I/O interface 608 includes hardware, software, or both, providing one or more interfaces for communication between computational metabolomics computing system 600 and one or more I/O devices. Computational metabolomics computing system 600 may include one or more of these I/O devices, where appropriate. One or more of these I/O devices may enable communication between a person and computational metabolomics computing system 600. As an example, and not by way of limitation, an I/O device may include a keyboard, keypad, microphone, monitor, mouse, printer, scanner, speaker, still camera, stylus, tablet, touch screen, trackball, video camera, another suitable I/O device or a combination of two or more of these. An I/O device may include one or more sensors. This disclosure contemplates any suitable I/O devices and any suitable I/O interfaces 606 for them. Where appropriate, I/O interface 608 may include one or more device or software drivers enabling processor 602 to drive one or more of these I/O devices. I/O interface 608 may include one or more I/O interfaces 606, where appropriate. Although this disclosure describes and illustrates a particular I/O interface, this disclosure contemplates any suitable I/O interface.

In certain embodiments, communication interface 610 includes hardware, software, or both providing one or more interfaces for communication (such as, for example, packet-based communication) between computational metabolomics computing system 600 and one or more other computer systems 600 or one or more networks. As an example, and not by way of limitation, communication interface 610 may include a network interface controller (NIC) or network adapter for communicating with an Ethernet or other wire-based network or a wireless NIC (WNIC) or wireless adapter for communicating with a wireless network, such as a WI-FI network. This disclosure contemplates any suitable network and any suitable communication interface 610 for it.

As an example, and not by way of limitation, computational metabolomics computing system 600 may communicate with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, computational metabolomics computing system 600 may communicate with a wireless PAN (WPAN) (such as, for example, a BLUETOOTH WPAN), a WI-FI network, a WI-MAX network, a cellular telephone network (such as, for example, a Global System for Mobile Communications (GSM) network), or other suitable wireless network or a combination of two or more of these. Computational metabolomics computing system 600 may include any suitable communication interface 610 for any of these networks, where appropriate. Communication interface 610 may include one or more communication interfaces 610, where appropriate. Although this disclosure describes and illustrates a particular communication interface, this disclosure contemplates any suitable communication interface.

In certain embodiments, bus 612 includes hardware, software, or both coupling components of computational metabolomics computing system 600 to each other. As an example, and not by way of limitation, bus 612 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or another suitable bus or a combination of two or more of these. Bus 612 may include one or more buses 612, where appropriate. Although this disclosure describes and illustrates a particular bus, this disclosure contemplates any suitable bus or interconnect.

Herein, a computer-readable non-transitory storage medium or media may include one or more semiconductor-based or other integrated circuits (ICs) (such, as for example, field-programmable gate arrays (FPGAs) or application-specific ICs (ASICs)), hard disk drives (HDDs), hybrid hard drives (HHDs), optical discs, optical disc drives (ODDs), magneto-optical discs, magneto-optical drives, floppy diskettes, floppy disk drives (FDDs), magnetic tapes, solid-state drives (SSDs), RAM-drives, SECURE DIGITAL cards or drives, any other suitable computer-readable non-transitory storage media, or any suitable combination of two or more of these, where appropriate. A computer-readable non-transitory storage medium may be volatile, non-volatile, or a combination of volatile and non-volatile, where appropriate.

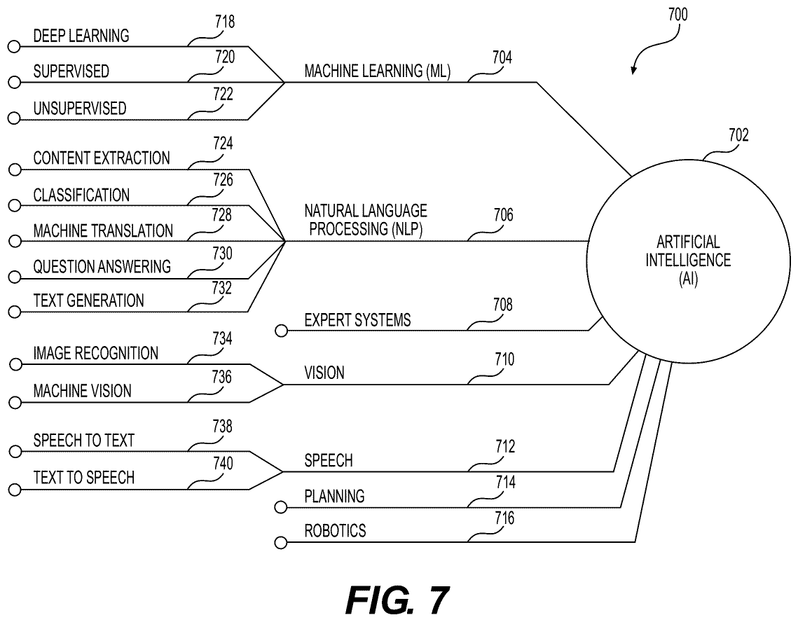

FIG. 7 illustrates a diagram 700 of an example artificial intelligence (AI) architecture 702 (e.g., which may be included as part of the computational metabolomics computing system 600) that may be utilized to generate predictions of the chemical structure or chemical properties of molecules, compounds, and small molecules (e.g., metabolites) based on MS data, in accordance with the presently disclosed embodiments. In certain embodiments, the AI architecture 702 may be implemented utilizing, for example, one or more processing devices that may include hardware (e.g., a general purpose processor, a graphic processing unit (GPU), an application-specific integrated circuit (ASIC), a system-on-chip (SoC), a microcontroller, a field-programmable gate array (FPGA), a central processing unit (CPU), an application processor (AP), a visual processing unit (VPU), a neural processing unit (NPU), a neural decision processor (NDP), a deep learning processor (DLP), a tensor processing unit (TPU), or any other processing device(s) that may be suitable for processing genomics data, metabolomics data, proteomics data, metagenomics data, transcriptomics data, and/or various other omics data), software (e.g., instructions running/executing on one or more processing devices), firmware (e.g., microcode), or some combination thereof.

In certain embodiments, as depicted by FIG. 6, the AI architecture 702 may include machine learning (ML) algorithms and functions 704, natural language processing (NLP) algorithms and functions 706, expert systems 708, computer-based vision algorithms and functions 710, speech recognition algorithms and functions 712, planning algorithms and functions 714, and robotics algorithms and functions 716. In certain embodiments, the ML algorithms and functions 704 may include any statistics-based algorithms that may be suitable for finding patterns across large amounts of data (e.g., “Big Data” such as genomics data, proteomics data, metabolomics data, metagenomics data, transcriptomics data, and/or various other omics data). For example, in certain embodiments, the ML algorithms and functions 704 may include deep learning algorithms 718, supervised learning algorithms 720, and unsupervised learning algorithms 722.

In certain embodiments, the deep learning algorithms 718 may include any artificial neural networks (ANNs) that may be utilized to learn deep levels of representations and abstractions from large amounts of data. For example, the deep learning algorithms 718 may include ANNs, such as a multilayer perceptron (MLP), an autoencoder (AE), a convolution neural network (CNN), a recurrent neural network (RNN), long short term memory (LSTM), a grated recurrent unit (GRU), a restricted Boltzmann Machine (RBM), a deep belief network (DBN), a bidirectional recurrent deep neural network (BRDNN), a generative adversarial network (GAN), and deep Q-networks, a neural autoregressive distribution estimation (NADE), an adversarial network (AN), attentional models (AM), a spiking neural network (SNN), deep reinforcement learning, and so forth.

In certain embodiments, the supervised learning algorithms 720 may include any algorithms that may be utilized to apply, for example, what has been learned in the past to new data using labeled examples for predicting future events. For example, starting from the analysis of a known training data set, the supervised learning algorithms 720 may produce an inferred function to make predictions about the output values. The supervised learning algorithms 720 may also compare its output with the correct and intended output and find errors in order to modify the supervised learning algorithms 720 accordingly. On the other hand, the unsupervised learning algorithms 722 may include any algorithms that may applied, for example, when the data used to train the unsupervised learning algorithms 722 are neither classified nor labeled. For example, the unsupervised learning algorithms 722 may study and analyze how systems may infer a function to describe a hidden structure from unlabeled data.

In certain embodiments, the NLP algorithms and functions 706 may include any algorithms or functions that may be suitable for automatically manipulating natural language, such as speech and/or text. For example, in some embodiments, the NLP algorithms and functions 706 may include content extraction algorithms or functions 724, classification algorithms or functions 726, machine translation algorithms or functions 728, question answering (QA) algorithms or functions 730, and text generation algorithms or functions 732. In certain embodiments, the content extraction algorithms or functions 724 may include a means for extracting text or images from electronic documents (e.g., webpages, text editor documents, and so forth) to be utilized, for example, in other applications.

In certain embodiments, the classification algorithms or functions 726 may include any algorithms that may utilize a supervised learning model (e.g., logistic regression, naïve Bayes, stochastic gradient descent (SGD), k-nearest neighbors, decision trees, random forests, support vector machine (SVM), and so forth) to learn from the data input to the supervised learning model and to make new observations or classifications based thereon. The machine translation algorithms or functions 728 may include any algorithms or functions that may be suitable for automatically converting source text in one language, for example, into text in another language. Indeed, in certain embodiments, the machine translation algorithms or functions 728 may be suitable for performing any of various language translation, text string based translation, or textual representation translation applications. The QA algorithms or functions 730 may include any algorithms or functions that may be suitable for automatically answering questions posed by humans in, for example, a natural language, such as that performed by voice-controlled personal assistant devices. The text generation algorithms or functions 732 may include any algorithms or functions that may be suitable for automatically generating natural language texts.

In certain embodiments, the expert systems 708 may include any algorithms or functions that may be suitable for simulating the judgment and behavior of a human or an organization that has expert knowledge and experience in a particular field (e.g., stock trading, medicine, sports statistics, and so forth). The computer-based vision algorithms and functions 710 may include any algorithms or functions that may be suitable for automatically extracting information from images (e.g., photo images, video images). For example, the computer-based vision algorithms and functions 710 may include image recognition algorithms 734 and machine vision algorithms 736. The image recognition algorithms 734 may include any algorithms that may be suitable for automatically identifying and/or classifying objects, places, people, and so forth that may be included in, for example, one or more image frames or other displayed data. The machine vision algorithms 736 may include any algorithms that may be suitable for allowing computers to “see”, or, for example, to rely on image sensors or cameras with specialized optics to acquire images for processing, analyzing, and/or measuring various data characteristics for decision making purposes.

In certain embodiments, the speech recognition algorithms and functions 712 may include any algorithms or functions that may be suitable for recognizing and translating spoken language into text, such as through automatic speech recognition (ASR), computer speech recognition, speech-to-text (STT) 738, or text-to-speech (TTS) 740 in order for the computing to communicate via speech with one or more users, for example. In certain embodiments, the planning algorithms and functions 714 may include any algorithms or functions that may be suitable for generating a sequence of actions, in which each action may include its own set of preconditions to be satisfied before performing the action. Examples of AI planning may include classical planning, reduction to other problems, temporal planning, probabilistic planning, preference-based planning, conditional planning, and so forth. Lastly, the robotics algorithms and functions 716 may include any algorithms, functions, or systems that may enable one or more devices to replicate human behavior through, for example, motions, gestures, performance tasks, decision-making, emotions, and so forth.

Herein, “or” is inclusive and not exclusive, unless expressly indicated otherwise or indicated otherwise by context. Therefore, herein, “A or B” means “A, B, or both,” unless expressly indicated otherwise or indicated otherwise by context. Moreover, “and” is both joint and several, unless expressly indicated otherwise or indicated otherwise by context. Therefore, herein. “A and B” means “A and B, jointly or severally,” unless expressly indicated otherwise or indicated otherwise by context.

Herein, “automatically” and its derivatives means “without human intervention,” unless expressly indicated otherwise or indicated otherwise by context.

### Embodiments

Among the provided embodiments are:

1. A method for identifying a chemical structure of a compound based on mass spectrometry (MS) data, the method comprising, by one or more computing devices:
receiving mass spectrometry (MS) data, wherein the MS data comprises a plurality of mass-to-charge values associated with fragments obtained from mass spectrometry performed on the compound;
inputting the plurality of mass-to-charge values into a tokenizer trained to generate a plurality of tokens based on the plurality of mass-to-charge values, wherein each of the plurality of tokens comprises a subset of the plurality of mass-to-charge values; and
determining one or more chemical structures of the compound based at least in part on the plurality of tokens.

2. The method of Embodiment 1 or Embodiment 2, wherein the MS data comprises a plurality of mass-to-charge values obtained from tandem mass spectrometry (MS2) performed on the compound.

3. The method of any one of Embodiments 1-2, wherein the MS data comprises a plurality of mass-to-charge values obtained from ion mobility mass spectrometry (IM-MS) performed on the compound.

4. The method of any one of Embodiments 1-3, wherein the plurality of mass-to-charge values comprises a sequence of spectral peaks corresponding to the plurality of mass-to-charge values.

5. The method of any one of Embodiments 1-4, wherein determining the one or more chemical structures of the compound comprises generating a deep simplified molecular-input line-entry system (DeepSMILES) strings based on the plurality of tokens.

6. The method of any one of Embodiments 1-5, wherein determining the one or more chemical structures of the compound comprises generating one or more self-referencing embedded strings (SELFIES).

7. The method of any one of Embodiments 1-6, wherein determining the one or more chemical structures of the compound comprises generating a simplified molecular-input line-entry system (SMILES) string.

8. The method of any one of Embodiments 1-7, further comprising:
generating a text string based on the plurality of mass-to-charge values, wherein the text string comprises a textual representation of the plurality of mass-to-charge values; and
inputting the text string into a tokenizer trained to generate a plurality of tokens based on the text string, wherein each of the plurality of tokens comprises a substring of data included in the text string.

9. The method of Embodiment 8, wherein the tokenizer comprises a subword tokenizer trained to generate the plurality of tokens based on a frequency of occurrence of one or more of the plurality of mass-to-charge values.

10. The method of Embodiment 9, wherein the subword tokenizer comprises a byte pair encoding (BPE) tokenizer trained to:
tokenize the plurality of mass-to-charge values into individual base vocabulary characters; and
iteratively determine a highest frequency of occurrence of pairs of the individual base vocabulary characters to be stored as respective tokens in a first vocabulary together with the individual base vocabulary characters until a predetermined vocabulary size is reached.

11. The method of Embodiment 10, wherein the first vocabulary is associated with the BPE tokenizer.

12. The method of any one of Embodiments 10-11, wherein the BPE tokenizer was trained by:
accessing a dataset of mass-to-charge values;
inputting the dataset of mass-to-charge values into the BPE tokenizer to identify a frequent occurrence of one or more subsets of sequential characters included in the dataset of mass-to-charge values;
generating, utilizing the BPE tokenizer, a second plurality of tokens based on the identified frequent occurrence of the one or more subsets of sequential characters included in the dataset of mass-to-charge values, wherein each of the second plurality of tokens corresponds to a respective one of the identified frequent occurrence of the one or more subsets of sequential characters; and
storing the second plurality of tokens to the first vocabulary.

13. The method of Embodiment 9, wherein the subword tokenizer comprises a WordPiece tokenizer trained to:
tokenize the plurality of mass-to-charge values string into individual base vocabulary characters; and
iteratively determine a most probable pair of the individual base vocabulary characters to be stored as respective tokens in a second vocabulary together with the individual base vocabulary characters until a predetermined vocabulary size is reached.

14. The method of Embodiment 13, wherein the second vocabulary is associated with the WordPiece tokenizer.

15. The method of any one of Embodiments 13-14, wherein the WordPiece tokenizer was trained by:
accessing a dataset of mass-to-charge values;
inputting the dataset of mass-to-charge values into the WordPiece tokenizer to identify one or more probable pairs of sequential characters included in the dataset of mass-to-charge values;
generating, utilizing the WordPiece tokenizer, a third plurality of tokens based on the identified one or more probable pairs of sequential characters, wherein each of the third plurality of tokens corresponds to a respective one of the identified one or more probable pairs of sequential characters; and
storing the third plurality of tokens to the second vocabulary.

16. The method of any one of Embodiments 9-15, wherein the subword tokenizer comprises a Unigram tokenizer trained to:
tokenize the plurality of mass-to-charge values into individual base vocabulary characters; and
iteratively determine a highest frequency of occurrence of pairs of the individual base vocabulary characters to be stored as respective tokens in a fifth vocabulary together with the individual base vocabulary characters; and
iteratively removing from the fifth vocabulary one or more of a pair of the individual base vocabulary characters based on a calculated loss associated therewith.

17. The method of Embodiment 16, wherein the Unigram tokenizer was trained by:
accessing a dataset of mass-to-charge values;
inputting the dataset of mass-to-charge values into the Unigram tokenizer to identify individual base vocabulary characters or one or more sequential characters included in the dataset of mass-to-charge values;
generating, utilizing the Unigram tokenizer, a fourth plurality of tokens based on the identified individual base vocabulary characters, wherein each of the fourth plurality of tokens corresponds to a respective one of the identified individual base vocabulary characters or the or one or more sequential characters; and
storing the fourth plurality of tokens to the third vocabulary.

18. The method of Embodiment 8, wherein the subword tokenizer comprises a byte pair encoding (BPE) dropout tokenizer trained to:
tokenize the plurality of mass-to-charge values into one or more subsets of values and individual base vocabulary characters to be stored as respective tokens in a third vocabulary associated with the Unigram tokenizer; and
iteratively removing from the third vocabulary one or more of a pair of the individual base vocabulary characters or one or more of a pair of the individual base vocabulary characters and the one or more subsets of values based on a calculated loss associated therewith

19. The method of any one of Embodiments 1-18, wherein the plurality of mass-to-charge values comprises a binning of the plurality of mass-to-charge values.

20. The method of Embodiment 19, wherein the binning of the plurality of mass-to-charge values comprises binning mass-to-charge (m/z) values of a sequence of spectral peaks corresponding to the plurality of mass-to-charge values.

21. The method of any one of Embodiments 19-20, wherein the binning of the plurality of mass-to-charge values comprises binning a sequence of spectral peaks corresponding to the plurality of mass-to-charge values in accordance with a predetermined precision value.

22. The method of any one of Embodiments 1-21, wherein the plurality of mass-to-charge values comprises a clustering of the plurality of mass-to-charge values.

23. The method of Embodiment 22, wherein the clustering of the plurality of mass-to-charge values comprises a hierarchical clustering.

24. The method of any one of Embodiments 22-23, wherein the clustering of the plurality of mass-to-charge values comprises a k-means clustering.

25. The method of any one of Embodiments 22-24, wherein the clustering of the plurality of mass-to-charge values is performed in one dimension by binning mass-to-charge (m/z) values of a sequence of spectral peaks corresponding to the plurality of mass-to-charge values.

26. The method of Embodiment 25, wherein the clustering of the plurality of mass-to-charge values is performed in two dimensions, wherein, for each of a sequence of spectral peaks corresponding to the plurality of mass-to-charge values, a first dimension of the two dimensions is an integer mass-to-charge (m/z) value and a second dimension of the two dimensions is a fraction m/z value.

27. The method of any one of Embodiments 1-26, further comprising:
prior to determining the one or more chemical structures of the compound, inputting the plurality of tokens into a transformer-based machine-learning model trained to generate a prediction of the one or more chemical structures based on the plurality of tokens.

28. The method of Embodiment 27, wherein determining the one or more chemical structures of the compound comprises outputting, by the transformer-based machine-learning model, one or more simplified molecular-input line-entry system (SMILES) strings representative of the one or more chemical structures.

29. A system including one or more computing devices, comprising:
one or more non-transitory computer-readable storage media including instructions; and
one or more processors coupled to the one or more storage media, the one or more processors configured to execute the instructions to:
   receive mass spectrometry (MS) data, wherein the MS data comprises a plurality of mass-to-charge values associated with fragments obtained from mass spectrometry performed on the compound;
   input the plurality of mass-to-charge values into a tokenizer trained to generate a plurality of tokens based on the plurality of mass-to-charge values, wherein each of the plurality of tokens comprises a subset of the plurality of mass-to-charge values; and
   determine one or more chemical structures of the compound based at least in part on the plurality of tokens.

30. A non-transitory computer-readable medium comprising instructions that, when executed by one or more processors of one or more computing devices, cause the one or more processors to:
receive mass spectrometry (MS) data, wherein the MS data comprises a plurality of mass-to-charge values associated with fragments obtained from mass spectrometry performed on the compound;
input the plurality of mass-to-charge values into a tokenizer trained to generate a plurality of tokens based on the plurality of mass-to-charge values, wherein each of the plurality of tokens comprises a subset of the plurality of mass-to-charge values; and
determine one or more chemical structures of the compound based at least in part on the plurality of tokens.

31. A method for identifying a chemical structure of a compound based on mass spectrometry (MS) data, the method comprising, by one or more computing devices:
receiving mass spectrometry (MS) data, wherein the MS data comprises a plurality of mass-to-charge values associated with fragments obtained from mass spectrometry performed on the compound;
generating a plurality of tokens based on the plurality of mass-to-charge values;
inputting the plurality of tokens into a bidirectional transformer-based machine-learning model trained to generate one or more predictions of a chemical structure of the compound based on the plurality of tokens; and
outputting, by the bidirectional transformer-based machine-learning model, the one or more predictions of the chemical structure of the compound.

32. The method of Embodiment 31, wherein the one or more predictions of the chemical structure of the compound comprises a plurality of candidates of the chemical structure of the compound.

33. The method of Embodiment 31 or Embodiment 32, wherein the bidirectional transformer-based machine-learning model comprises a bidirectional and auto-regressive transformer (BART) model.

34. The method of any one of Embodiments 31-33, wherein the bidirectional transformer-based machine-learning model comprises a bidirectional encoder representations for transformer (BERT) model.

35. The method of any one of Embodiments 31-34, wherein the bidirectional transformer-based machine-learning model comprises a generative pre-trained transformer (GPT) model.

36. The method of any one of Embodiments 31-35, further comprising generating an image of the plurality of candidates of the chemical structure of the compound.

37. The method of any one of Embodiments 31-36, wherein the mass spectrometry comprises a tandem mass spectrometry technique.

38. The method of any one of Embodiments 31-37, wherein the mass spectrometry is an electrospray ionization mass spectrometry technique.

39. The method of Embodiment 38, wherein the electrospray ionization mass spectrometry technique comprises a positive-ion mode mass spectrometry technique.

40. The method of Embodiment 39, wherein the electrospray ionization mass spectrometry technique comprises a negative-ion mode mass spectrometry technique.

41. The method of any one of Embodiments 31-40, wherein the mass spectrometry comprises use of a data-dependent acquisition technique.

42. The method of any one of Embodiments 31-40, wherein the mass spectrometry technique comprises use of a data-independent acquisition technique.

43. The method of any one of Embodiments 31-42, wherein the mass spectrometry comprises use of a mass spectrometer.

44. The method of Embodiment 43, wherein the mass spectrometer has a mass accuracy of 25 ppm or greater.

45. The method of any one of Embodiments 31-44, wherein the mass spectrometry comprises an upstream separation technique.

46. The method of Embodiment 45, wherein the separation technique is a liquid chromatography technique.

47. The method of Embodiment 46, wherein the liquid chromatography technique is an online liquid chromatography technique.

48. The method of any one of Embodiments 31-47, further comprising subjecting a sample comprising the compound to mass spectrometry to generate the MS data.

49. The method of Embodiment 48, further comprising obtaining the sample.

50. The method of Embodiment 48 or 49, wherein the sample is a natural sample or a derivative thereof.

51. The method of any one of Embodiments 31-50, wherein the sample comprises a plant extract or a derivative thereof.

52. The method of any one of Embodiments 31-51, wherein the compound is a small molecule having a molecular weight of less than 2,000 Dalton (da).

53. The method of any one of Embodiments 31-52, wherein the compound is a natural product.

54. A system including one or more computing devices, comprising:
one or more non-transitory computer-readable storage media including instructions; and
one or more processors coupled to the one or more storage media, the one or more processors configured to execute the instructions to:
   receive mass spectrometry (MS) data, wherein the MS data comprises a plurality of mass-to-charge values associated with fragments obtained from mass spectrometry performed on the compound;
   generate a plurality of tokens based on the plurality of mass-to-charge values;
   input the plurality of tokens into a bidirectional transformer-based machine-learning model trained to generate one or more predictions of a chemical structure of the compound based on the plurality of tokens; and
   output, by the bidirectional transformer-based machine-learning model, the one or more predictions of the chemical structure of the compound.

55. A non-transitory computer-readable medium comprising instructions that, when executed by one or more processors of one or more computing devices, cause the one or more processors to:
receive mass spectrometry (MS) data, wherein the MS data comprises a plurality of mass-to-charge values associated with fragments obtained from mass spectrometry performed on the compound;
generate a plurality of tokens based on the plurality of mass-to-charge values;
input the plurality of tokens into a bidirectional transformer-based machine-learning model trained to generate one or more predictions of a chemical structure of the compound based on the plurality of tokens; and
output, by the bidirectional transformer-based machine-learning model, the one or more predictions of the chemical structure of the compound.

56. A method for training a transformer-based machine-learning model to identify a chemical structure of a compound based on a mass spectrometry (MS) data, the method comprising, by one or more computing devices:
accessing a data set of mass spectra data, wherein the data set of mass spectra data comprises a plurality of mass-to-charge values corresponding to a compound;
generating a plurality of tokens based on the plurality of mass-to-charge values, wherein the plurality of tokens comprises a set of one or more corrupted tokens and uncorrupted tokens; and
inputting the plurality of tokens into the transformer-based machine-learning model to generate a prediction of the one or more corrupted tokens based on the uncorrupted tokens, the prediction of the one or more corrupted tokens corresponding to an original sequence of tokens representative of the plurality of mass-to-charge values.

57. The method of Embodiment 56, wherein the transformer-based machine-learning model is further trained by:
computing a cross-entropy loss value based on a comparison of the prediction of the one or more corrupted tokens and the original sequence of tokens representative of the plurality of mass-to-charge values; and
updating the transformer-based machine-learning model based on the cross-entropy loss value.

58. The method of Embodiment 57, wherein training the transformer-based machine-learning model comprises pre-training the transformer-based machine-learning model, the method further comprising:
fine-tuning the pre-trained transformer-based machine-learning model.

59. The method of Embodiment 58, wherein fine-tuning the pre-trained transformer-based machine-learning model comprises:
accessing a second data set of mass spectra data, wherein the second data second set of mass spectra data comprises a second plurality of mass-to-charge values corresponding to a compound;
generating a second plurality of tokens based on the second plurality of mass-to-charge values; and
inputting the second plurality of tokens into the pre-trained transformer-based machine-learning model to generate a prediction of one or more chemical structures of the compound based on the second plurality of tokens.

60. The method of Embodiment 59, wherein the fine-tuned transformer-based machine-learning model is further trained by:
computing a second cross-entropy loss value based on a comparison of the prediction of the one or more chemical structures and a second original sequence of tokens corresponding to the second plurality of mass-to-charge values; and
updating the fine-tuned transformer-based machine-learning model based on the second cross-entropy loss value.

61. The method of any one of Embodiments 59-60, wherein the prediction of the one or more chemical structures comprises one or more deep simplified molecular-input line-entry system (DeepSMILES) strings.

62. The method of any one of Embodiments 59-61, wherein the prediction of the one or more chemical structures comprises one or more simplified molecular-input line-entry system (SMILES) strings.

63. The method of any one of Embodiments 59-62, wherein the prediction of the one or more chemical structures comprises one or more self-referencing embedded strings (SELFIES).

64. The method of any one of Embodiments 56-63, wherein the transformer-based machine-learning model is further trained by:
accessing a dataset of mass spectra data, wherein the dataset of mass spectra data comprises a second plurality of mass-to-charge values each associated with a predetermined chemical data, and wherein the predetermined chemical data comprises a start-of-sequence token for contextualizing one or more tokens to be generated based on the second plurality of mass-to-charge values;
generating a second plurality of tokens based on the second plurality of mass-to-charge values and the associated predetermined chemical data, wherein the second plurality of tokens comprises a set of one or more corrupted tokens and uncorrupted tokens; and
inputting the second plurality of tokens into the transformer-based machine-learning model to generate a prediction of the one or more corrupted tokens based on the uncorrupted tokens and the associated predetermined chemical data, the prediction of the one or more corrupted tokens corresponding to a prediction of a plurality of candidates of the chemical structure of the compound.

65. The method of Embodiment 64, wherein the predetermined chemical data comprises a chemical formula.

66. The method of any one of Embodiments 64-65, wherein the predetermined chemical data comprises a representation of a chemical structural property.

67. The method of any one of Embodiments 56-66, wherein the transformer-based machine-learning model was trained by:
accessing a dataset of mass spectra data, wherein the dataset of mass spectra data comprises a second plurality of mass-to-charge values corresponding to one or more compounds having an undetermined chemical structure;
generating a second plurality of tokens based on the second plurality of mass-to-charge values, wherein the second plurality of tokens comprises a set of one or more corrupted tokens and uncorrupted tokens;
determining a contextual data associated with the set of one or more corrupted tokens and uncorrupted tokens; and
inputting the second plurality of tokens into the transformer-based machine-learning model to generate a prediction of the one or more corrupted tokens based on the uncorrupted tokens and the contextual data, the prediction of the one or more corrupted tokens corresponding to a prediction of a plurality of candidates a chemical structure of the one or more compounds and a chemical formula associated with the one or more compounds.

68. The method of any one of Embodiments 56-67, wherein each of the plurality of mass-to-charge values includes a respective intensity value, the method further comprising:
prior to generating the plurality of tokens, ordering the plurality of mass-to-charge values into a sequence of least to greatest based on the respective intensity value.

69. The method of any one of Embodiments 56-68, wherein the MS data comprises a sequence of charged fragments ordered from least intensity to greatest intensity;
generating a second plurality of tokens based on the ordered sequence of charged fragments, wherein a position encoding of each token of the second plurality of tokens is representative of an intensity of a charged fragment corresponding to the token; and
inputting the second plurality of tokens into a transformer-based machine-learning model trained to generate a prediction of one or more chemical structures of the compound based at least in part on the second plurality of tokens and the position encoding.

70. The method of any one of Embodiments 56-69, wherein inputting the plurality of tokens into the transformer-based machine-learning model further comprises:
inputting the plurality of tokens into an embedding layer configured to encode the plurality of tokens into a vector representation, wherein the vector representation is utilized to contextualize each of the plurality of tokens; and
modifying at least a subset of the vector representation to include an intensity value for each charged fragment corresponding to the plurality of tokens.

71. The method of any one of Embodiments 56-70, wherein the MS data comprises a plurality of mass-to-charge values obtained from tandem mass spectrometry (MS2) performed on the compound.

72. The method of any one of Embodiments 56-71, wherein the MS data comprises a plurality of mass-to-charge values obtained from ion mobility mass spectrometry (IM-MS) performed on the compound.

73. The method of any one of Embodiments 56-72, wherein the plurality of tokens comprises one or more masked tokens and unmasked tokens, the method further comprising:
inputting the second plurality of tokens into the transformer-based machine-learning model to generate a prediction of the one or more masked tokens based on the unmasked tokens, the prediction of the one or more masked tokens corresponding to the prediction of the plurality of candidates of the chemical structure of the compound.

74. The method of any one of Embodiments 56-73, further comprising performing a process to corrupt the one or more corrupted tokens included in the set of one or more corrupted tokens and uncorrupted tokens.

75. The method of Embodiment 74, wherein the process to corrupt the one or more corrupted tokens comprises a process to corrupt one or more random spectral peaks in a sequence of spectral peaks corresponding to the plurality of mass-to-charge values.

76. The method of any one of Embodiments 74-75, wherein the process to corrupt the one or more corrupted tokens comprises a process to corrupt one or more high-intensity spectral peaks in a sequence of spectral peaks corresponding to the plurality of mass-to-charge values.

77. The method of any one of Embodiments 74-76, wherein the process to corrupt the one or more corrupted tokens comprises a process to corrupt one or more subsequences of spectral peaks in a sequence of spectral peaks corresponding to the plurality of mass-to-charge values.

78. The method of any one of Embodiments 74-77, wherein the process to corrupt the one or more corrupted tokens comprises a process to reshuffle spectral peaks in a sequence of spectral peaks corresponding to the plurality of mass-to-charge values.

79. The method of any one of Embodiments 74-78, wherein the process to corrupt the one or more corrupted tokens comprises a token deletion process, a token masking process, a text infilling process, a text string permutation process, or a sequence rotation process.

80. The method of any one of Embodiments 56-79, wherein the transformer-based machine-learning model comprises a bidirectional transformer-based machine-learning model.

81. The method of any one of Embodiments 56-80, wherein the transformer-based machine-learning model comprises a bidirectional and auto-regressive transformer (BART) model.

82. The method of any one of Embodiments 56-81, wherein the transformer-based machine-learning model comprises a bidirectional encoder representations for transformer (BERT) model.

83. The method of any one of Embodiments 56-82, wherein the transformer-based machine-learning model comprises a generative pre-trained transformer (GPT) model.

84. The method of any one of Embodiments 56-83, wherein the transformer-based machine-learning model is further trained by:
accessing a dataset of small molecule data, wherein the dataset of small molecule data is not associated with MS data;
generating a set of text strings representative of the dataset of small molecule data; and
inputting the set of text strings into the transformer-based machine-learning model to generate a prediction of one or more chemical structures corresponding to the dataset of small molecule data.

85. The method of Embodiment 84, wherein the small molecule data comprises a molecule having a mass of 900 Dalton (da) or less.

86. The method of Embodiment 84 or Embodiment 85, wherein the small molecule data comprises a molecule having a mass of 700 Dalton (da) or less.

87. The method of any one of Embodiments 84-86, wherein the small molecule data comprises a molecule having a mass of 500 Dalton (da) or less.

88. The method of any one of Embodiments 84-87, wherein the small molecule data comprises a molecule having a mass of 300 Dalton (da) or less.

89. A system including one or more computing devices, comprising:
one or more non-transitory computer-readable storage media including instructions; and
one or more processors coupled to the one or more storage media, the one or more processors configured to execute the instructions to:
   access a data set of mass spectra data, wherein the data set of mass spectra data comprises a plurality of mass-to-charge values corresponding to a compound;
   generate a plurality of tokens based on the plurality of mass-to-charge values, wherein the plurality of tokens comprises a set of one or more corrupted tokens and uncorrupted tokens; and
   input the plurality of tokens into the transformer-based machine-learning model to generate a prediction of the one or more corrupted tokens based on the uncorrupted tokens, the prediction of the one or more corrupted tokens corresponding to an original sequence of tokens representative of the plurality of mass-to-charge values.

90. A non-transitory computer-readable medium comprising instructions that, when executed by one or more processors of one or more computing devices, cause the one or more processors to:
access a data set of mass spectra data, wherein the data set of mass spectra data comprises a plurality of mass-to-charge values corresponding to a compound;
generate a plurality of tokens based on the plurality of mass-to-charge values, wherein the plurality of tokens comprises a set of one or more corrupted tokens and uncorrupted tokens; and
input the plurality of tokens into the transformer-based machine-learning model to generate a prediction of the one or more corrupted tokens based on the uncorrupted tokens, the prediction of the one or more corrupted tokens corresponding to an original sequence of tokens representative of the plurality of mass-to-charge values.

91. A method for training a transformer-based machine-learning model to identify a chemical property of a compound based on a mass spectrometry (MS) data, the method comprising, by one or more computing devices:
receiving mass spectrometry (MS) data, wherein the MS data comprises a plurality of mass-to-charge values obtained from mass spectrometry performed on a compound;
generating a plurality of tokens based on the plurality of mass-to-charge values, wherein the plurality of tokens comprises a set of one or more masked tokens and unmasked tokens;
inputting the plurality of tokens into a transformer-based machine-learning model to generate a prediction of the one or more masked tokens based on the unmasked tokens; and
generating, by the transformer-based machine-learning model, the prediction of the one or more masked tokens, the prediction of the one or more masked tokens corresponding at least in part to a prediction of one or more chemical properties of the compound.

92. The method of Embodiment 91, wherein inputting the plurality of tokens into the transformer-based machine-learning model further comprises:
inputting the plurality of tokens into the transformer-based machine-learning model to generate a vector representation of the one or more masked tokens based on the unmasked tokens; and
inputting the vector representation of the one or more masked tokens into a feed forward neural network trained to generate a prediction of a subset of data corresponding to the one or more masked tokens.

93. The method of Embodiment 91 or Embodiment 92, wherein the transformer-based machine-learning model comprises a bidirectional encoder representations for transformer (BERT) model.

94. The method of any one of Embodiments 91-93, wherein the MS data comprises a plurality of mass-to-charge values obtained from tandem mass spectrometry (MS2) performed on the compound.

95. The method of any one of Embodiments 91-94, wherein the MS data comprises a plurality of mass-to-charge values obtained from ion mobility mass spectrometry (IM-MS) performed on the compound.

96. The method of any one of Embodiments 91-95, wherein training the transformer-based machine-learning model comprises pre-training the transformer-based machine-learning model, the method further comprising:

fine-tuning the pre-trained transformer-based machine-learning model to identify the chemical property of the compound.

97. The method of Embodiment 96, wherein the transformer-based machine-learning model is further trained by:
computing a loss value based on a comparison of the prediction of the one or more masked tokens and an input sequence of tokens corresponding to the plurality of mass-to-charge values; and
updating the transformer-based machine-learning model based on the computed loss value.

98. The method of Embodiment 97, wherein the transformer-based machine-learning model is associated with a predetermined vocabulary, and wherein the predetermined vocabulary comprises one or more sets of tokens corresponding to a curated dataset of experimental simplified molecular-input line-entry system (SMILES) strings.

99. The method of any one of Embodiments 91-98, wherein the set of one or more masked tokens comprises at least 15% of a total number of the plurality of tokens.

100. The method of any one of Embodiments 91-99, the prediction of the one or more chemical properties comprises a prediction of a natural product class of the compound.

101. The method of any one of Embodiments 91-100, the prediction of the one or more chemical properties comprises a prediction of a LogP value associated with the compound.

102. The method of any one of Embodiments 91-101, the prediction of the one or more chemical properties comprises a prediction of a number of hemoglobin receptors of the compound.

103. The method of any one of Embodiments 91-102, the prediction of the one or more chemical properties comprises a prediction of a number of hemoglobin donors of the compound.

104. The method of any one of Embodiments 91-103, the prediction of the one or more chemical properties comprises a prediction of a polar surface area of the compound.

105. The method of any one of Embodiments 91-104, the prediction of the one or more chemical properties comprises a prediction of a number of rotatable bonds of the compound.

106. The method of any one of Embodiments 91-105, the prediction of the one or more chemical properties comprises a prediction of a number of aromatic rings of the compound.

107. The method of any one of Embodiments 91-106, the prediction of the one or more chemical properties comprises a prediction of a number of aliphatic rings of the compound.

108. The method of any one of Embodiments 91-107, the prediction of the one or more chemical properties comprises a prediction of a number of heteroatoms of the compound.

109. The method of any one of Embodiments 91-108, the prediction of the one or more chemical properties comprises a prediction of a fraction of sp³ carbon atoms (Fsp³) of the compound.

110. The method of any one of Embodiments 91-109, the prediction of the one or more chemical properties comprises a prediction of a molecular weight of the compound.

111. The method of any one of Embodiments 91-110, the prediction of the one or more chemical properties comprises a prediction of an adduct or fragment associated with the compound.

112. A system including one or more computing devices, comprising:
one or more non-transitory computer-readable storage media including instructions; and
one or more processors coupled to the one or more storage media, the one or more processors configured to execute the instructions to:
   receive mass spectrometry (MS) data, wherein the MS data comprises a plurality of mass-to-charge values obtained from mass spectrometry performed on a compound;
   generate a plurality of tokens based on the plurality of mass-to-charge values, wherein the plurality of tokens comprises a set of one or more masked tokens and unmasked tokens;
   input the plurality of tokens into a transformer-based machine-learning model to generate a prediction of the one or more masked tokens based on the unmasked tokens; and
   generate, by the transformer-based machine-learning model, the prediction of the one or more masked tokens, the prediction of the one or more masked tokens corresponding at least in part to a prediction of one or more chemical properties of the compound.

113. A non-transitory computer-readable medium comprising instructions that, when executed by one or more processors of one or more computing devices, cause the one or more processors to:
receive mass spectrometry (MS) data, wherein the MS data comprises a plurality of mass-to-charge values obtained from mass spectrometry performed on a compound;
generate a plurality of tokens based on the plurality of mass-to-charge values, wherein the plurality of tokens comprises a set of one or more masked tokens and unmasked tokens;
input the plurality of tokens into a transformer-based machine-learning model to generate a prediction of the one or more masked tokens based on the unmasked tokens; and
generate, by the transformer-based machine-learning model, the prediction of the one or more masked tokens, the prediction of the one or more masked tokens corresponding at least in part to a prediction of one or more chemical properties of the compound.

114. A method for generating training data for a machine-learning model trained to identify of a chemical structure of a compound, the method comprising, by one or more computing devices:
accessing a first set of mass spectra data, wherein the first set of mass spectra data was obtained experimentally from a compound;
generating, by a first neural network of a generative adversarial network (GAN) model, a second set of mass spectra data;
inputting the first set of mass spectra data and the second set of mass spectra data into a second neural network of the GAN model, wherein the second neural network is trained to classify the first set of mass spectra data and the second set of mass spectra; and
generating a training data set based on the classification of the first set of mass spectra data and the second set of mass spectra.

115. The method of Embodiment 114, wherein the first neural network comprises a generator of the GAN model.

116. The method of Embodiment 114 or Embodiment 115, wherein the second neural network comprises a discriminator of the GAN model.

117. The method of any one of Embodiments 114-116, wherein the first neural network is trained to generate the second set of mass spectra data based on a random noise data.

118. The method of any one of Embodiments 114-117, further comprising generating a training data set based on the first set of mass spectra data and a third set of mass spectra data, wherein the third set of mass spectra data comprises padding data values configured to augment the first set of mass spectra data.

119. The method of Embodiment 118, wherein the third set of mass spectra data was obtained from a blank chemical sample compound.

120. The method of any one of Embodiments 114-119, further comprising:
calculating one or more loss functions based on the classification of the first set of mass spectra data and the second set of mass spectra; and
generating the training data set based on the first set of mass spectra data and the second set of mass spectra data when the one or more loss functions satisfies a predetermined criterion.

121. The method of any one of Embodiments 114-120, wherein the second set of mass spectra data comprises synthetic data.

122. The method of any one of Embodiments 114-121, wherein the first set of mass spectra data corresponds to a set of naturally-occurring molecules and the second set of mass spectra data corresponds to a set of non-naturally-occurring molecules, and wherein a number of the set of non-naturally-occurring molecules is greater than a number of the set of naturally-occurring molecules.

123. A system including one or more computing devices, comprising:
one or more non-transitory computer-readable storage media including instructions; and
one or more processors coupled to the one or more storage media, the one or more processors configured to execute the instructions to:
   access a first set of mass spectra data, wherein the first set of mass spectra data was obtained experimentally from a compound;
   generate, by a first neural network of a generative adversarial network (GAN) model, a second set of mass spectra data;
   input the first set of mass spectra data and the second set of mass spectra data into a second neural network of the GAN model, wherein the second neural network is trained to classify the first set of mass spectra data and the second set of mass spectra; and
   generate a training data set based on the classification of the first set of mass spectra data and the second set of mass spectra.

124. A non-transitory computer-readable medium comprising instructions that, when executed by one or more processors of one or more computing devices, cause the one or more processors to:
access a first set of mass spectra data, wherein the first set of mass spectra data was obtained experimentally from a compound;
generate, by a first neural network of a generative adversarial network (GAN) model, a second set of mass spectra data;
input the first set of mass spectra data and the second set of mass spectra data into a second neural network of the GAN model, wherein the second neural network is trained to classify the first set of mass spectra data and the second set of mass spectra; and
generate a training data set based on the classification of the first set of mass spectra data and the second set of mass spectra.

125. A method for training a byte pair encoding (BPE) tokenizer associated with identifying a chemical structure of a compound based on mass spectrometry (MS) data, the method comprising, by one or more computing devices:
accessing a data set of one or more simplified molecular-input line-entry system (SMILES) strings corresponding to a compound;
inputting the one or more SMILES strings into a byte pair encoding (BPE) tokenizer trained to 1) tokenize the one or more SMILES string into individual base characters, and 2) determine a highest frequency of occurrence of pairs of the individual base characters to be stored as respective tokens in a vocabulary together with the individual base characters; and
utilizing one or more of the respective tokens to determine one or more candidates of a chemical structure of the compound.

126. The method of Embodiment 125, wherein the BPE tokenizer is trained to iteratively determine the highest frequency of occurrence of pairs of the individual base characters to be stored as respective tokens in the vocabulary together with the individual base characters until a predetermined vocabulary size is reached.

127. The method of Embodiment 125 or Embodiment 126, wherein the vocabulary is associated with the BPE tokenizer.

128. The method of any one of Embodiments 125-127, wherein utilizing the one or more of the respective tokens to determine the one or more candidates of the chemical structure comprises:
inputting the plurality of tokens into a transformer-based machine-learning model trained to generate a prediction of the one or more chemical structures based on the one or more of the respective tokens.

129. The method of any one of Embodiments 125-128, wherein the one or more SMILES strings comprises one or more deep simplified molecular-input line-entry system (DeepSMILES) strings.

130. The method of any one of Embodiments 125-129, wherein the one or more SMILES strings comprises one or more self-referencing embedded strings (SELFIES).

131. A system including one or more computing devices, comprising:
one or more non-transitory computer-readable storage media including instructions; and
one or more processors coupled to the one or more storage media, the one or more processors configured to execute the instructions to:
   access a data set of one or more simplified molecular-input line-entry system (SMILES) strings corresponding to a compound;
   input the one or more SMILES strings into a byte pair encoding (BPE) tokenizer trained to 1) tokenize the one or more SMILES string into individual base characters, and 2) determine a highest frequency of occurrence of pairs of the individual base characters to be stored as respective tokens in a vocabulary together with the individual base characters; and
   utilize one or more of the respective tokens to determine one or more candidates of a chemical structure of the compound.

132. A non-transitory computer-readable medium comprising instructions that, when executed by one or more processors of one or more computing devices, cause the one or more processors to:
access a data set of one or more simplified molecular-input line-entry system (SMILES) strings corresponding to a compound;
input the one or more SMILES strings into a byte pair encoding (BPE) tokenizer trained to 1) tokenize the one or more SMILES string into individual base characters, and 2) determine a highest frequency of occurrence of pairs of the individual base characters to be stored as respective tokens in a vocabulary together with the individual base characters; and
utilize one or more of the respective tokens to determine one or more candidates of a chemical structure of the compound.

133. A method for training a transformer-based machine-learning model to identify a chemical structure of a compound based on a mass spectrometry (MS) data, the method comprising, by one or more computing devices:
accessing a data set of one or more simplified molecular-input line-entry system (SMILES) strings corresponding to a compound;
generating a plurality of tokens based on the one or more SMILES strings, wherein the plurality of tokens comprises a set of one or more corrupted tokens and uncorrupted tokens; and
inputting the plurality of tokens into the transformer-based machine-learning model to generate a prediction of the one or more corrupted tokens based on the uncorrupted tokens, the prediction of the one or more corrupted tokens corresponding to an original sequence of tokens representative of the one or more SMILES strings.

134. The method of Embodiment 133, wherein the transformer-based machine-learning model is further trained by:
computing a cross-entropy loss value based on a comparison of the prediction of the one or more corrupted tokens and the original sequence of tokens representative of the one or more SMILES strings; and
updating the transformer-based machine-learning model based on the cross-entropy loss value.

135. The method of Embodiment 134, wherein training the transformer-based machine-learning model comprises pre-training the transformer-based machine-learning model, the method further comprising:
fine-tuning the pre-trained transformer-based machine-learning model.

136. The method of Embodiment 135, wherein fine-tuning the pre-trained transformer-based machine-learning model comprises:
accessing a data set of mass spectra data, wherein the data second set of mass spectra data comprises a plurality of mass-to-charge values corresponding to a compound;
generating a second plurality of tokens based on the plurality of mass-to-charge values; and
inputting the second plurality of tokens into the pre-trained transformer-based machine-learning model to generate a prediction of one or more chemical structures of the compound based on the second plurality of tokens.

137. The method of Embodiment 136, wherein the fine-tuned transformer-based machine-learning model is further trained by:
computing a second cross-entropy loss value based on a comparison of the prediction of the one or more chemical structures and a second original sequence of tokens corresponding to the plurality of mass-to-charge values; and
updating the fine-tuned transformer-based machine-learning model based on the second cross-entropy loss value.

138. The method of Embodiment 136 or 137, wherein the prediction of the one or more chemical structures comprise one or more simplified molecular-input line-entry system (SMILES) strings.

139. The method of any one of Embodiments 136-138, wherein the prediction of the one or more chemical structures comprise one or more deep simplified molecular-input line-entry system (DeepSMILES) strings.

140. The method of any one of Embodiments 136-139, wherein the prediction of the one or more chemical structures comprise one or more self-referencing embedded strings (SELFIES).

141. A system including one or more computing devices, comprising:
one or more non-transitory computer-readable storage media including instructions; and
one or more processors coupled to the one or more storage media, the one or more processors configured to execute the instructions to:
   access a data set of one or more simplified molecular-input line-entry system (SMILES) strings corresponding to a compound;
   generate a plurality of tokens based on the one or more SMILES strings, wherein the plurality of tokens comprises a set of one or more corrupted tokens and uncorrupted tokens; and
   input the plurality of tokens into the transformer-based machine-learning model to generate a prediction of the one or more corrupted tokens based on the uncorrupted tokens, the prediction of the one or more corrupted tokens corresponding to an original sequence of tokens representative of the one or more SMILES strings.

142. A non-transitory computer-readable medium comprising instructions that, when executed by one or more processors of one or more computing devices, cause the one or more processors to:
access a data set of one or more simplified molecular-input line-entry system (SMILES) strings corresponding to a compound;
generate a plurality of tokens based on the one or more SMILES strings, wherein the plurality of tokens comprises a set of one or more corrupted tokens and uncorrupted tokens; and
input the plurality of tokens into the transformer-based machine-learning model to generate a prediction of the one or more corrupted tokens based on the uncorrupted tokens, the prediction of the one or more corrupted tokens corresponding to an original sequence of tokens representative of the one or more SMILES strings.

143. A method for identifying a chemical structure of a compound based on mass spectrometry (MS) data, the method comprising, by one or more computing devices:
receiving mass spectrometry (MS) data, wherein the MS data comprises a plurality of mass-to-charge values associated with fragments obtained from mass spectrometry performed on the compound;
generating a plurality of encodings based on the plurality of mass-to-charge values;
inputting the plurality of encodings into a bidirectional transformer-based machine-learning model trained to generate one or more predictions of a chemical structure of the compound based on the plurality of encodings; and
outputting, by the bidirectional transformer-based machine-learning model, the one or more predictions of the chemical structure of the compound.

144. A method for identifying a chemical structure of a compound based on mass spectrometry (MS) data, the method comprising, by one or more computing devices:
accessing a data set of mass spectra data, wherein the data set of mass spectra data comprises a plurality of mass-to-charge values corresponding to a compound;
generating a plurality of sinusoidal embeddings based on the plurality of mass-to-charge values;
inputting the plurality of sinusoidal embeddings into a transformer-based machine-learning model trained to generate a prediction of the chemical structure of a compound based at least in part on the plurality of sinusoidal embeddings; and
generating the prediction of the chemical structure of the compound based at least in part on the plurality of sinusoidal embeddings.

145. The method of Embodiment 144, wherein generating the plurality of sinusoidal embeddings comprises encoding the plurality of mass-to-charge values into one or more fixed vector representations.

146. The method of Embodiment 144 or Embodiment 145, wherein generating the plurality of sinusoidal embeddings comprises encoding the plurality of mass-to-charge values based on one or more sinusoidal functions.

147. The method of Embodiment 146, wherein the one or more sinusoidal functions comprise a sine function, a cosine function, or a combination thereof.

148. The method of Embodiment 146 or Embodiment 147, wherein the one or more sinusoidal functions is expressed as:

$$SE ( m / z , 2 i ; d ) = sin ( 2 {π [ {{λ}_{min} ( \frac{{λ}_{max}}{{λ}_{min}} )}^{2 i / ( d - 2 )} ]}^{- 1} m / z ) \\ SE ( m / z , 2 i + 1 ; d ) = cos ( 2 {π [ {{λ}_{min} ( \frac{{λ}_{max}}{{λ}_{min}} )}^{2 i / ( d - 2 )} ]}^{- 1} m / z ) .$$

149. A method for identifying a chemical structure of a compound based on mass spectrometry (MS) data, the method comprising, by one or more computing devices:
receiving mass spectrometry (MS) data, wherein the MS data comprises a plurality of mass-to-charge values and a precursor mass associated with a compound;
generating a plurality of tokens based at least in part on the plurality of mass-to-charge values and precursor mass;
inputting the plurality of tokens into a bidirectional transformer-based machine-learning model trained to generate one or more predictions of a chemical structure of the compound based on the plurality of tokens; and
outputting, by the bidirectional transformer-based machine-learning model, the one or more predictions of the chemical structure of the compound.

150. The method of Embodiment 149, wherein the one or more predictions of the chemical structure of the compound comprises a plurality of candidates of the chemical structure of the compound.

151. The method of Embodiment 149 or Embodiment 150, wherein the bidirectional transformer-based machine-learning model comprises a bidirectional and auto-regressive transformer (BART) model.

152. The method of Embodiment 149, wherein the bidirectional transformer-based machine-learning model comprises a bidirectional encoder representations for transformer (BERT) model.

153. The method of Embodiment 149, wherein the bidirectional transformer-based machine-learning model comprises a generative pre-trained transformer (GPT) model.

154. The method of Embodiment 1149-153, further comprising generating an image of the plurality of candidates of the chemical structure of the compound.

155. The method of any one of Embodiments 149-154, wherein the mass spectrometry comprises a tandem mass spectrometry technique.

156. The method of any one of Embodiments 149-155, wherein the mass spectrometry is an electrospray ionization mass spectrometry technique.

157. The method of Embodiment 156, wherein the electrospray ionization mass spectrometry technique comprises a positive-ion mode mass spectrometry technique.

158. The method of Embodiment 157, wherein the electrospray ionization mass spectrometry technique comprises a negative-ion mode mass spectrometry technique.

159. The method of any one of Embodiments 149-158, wherein the mass spectrometry comprises use of a data-dependent acquisition technique.

160. The method of any one of Embodiments 1149-159, wherein the mass spectrometry technique comprises use of a data-independent acquisition technique.

161. The method of any one of Embodiments 149-160, wherein the mass spectrometry comprises use of a mass spectrometer.

162. The method of Embodiment 161, wherein the mass spectrometer has a mass accuracy of 25 ppm or greater.

163. The method of any one of Embodiments 149-162, wherein the mass spectrometry comprises an upstream separation technique.

164. The method of Embodiment 163, wherein the separation technique is a liquid chromatography technique.

165. The method of Embodiment 164, wherein the liquid chromatography technique is an online liquid chromatography technique.

166. The method of any one of Embodiments 149-165, further comprising subjecting a sample comprising the compound to mass spectrometry to generate the MS data.

167. The method of Embodiment 166, further comprising obtaining the sample.

168. The method of Embodiment 166 or 167, wherein the sample is a natural sample or a derivative thereof.

169. The method of any one of Embodiments 149-168, wherein the sample comprises a plant extract or a derivative thereof.

170. The method of any one of Embodiments 149-169, wherein the compound is a small molecule having a molecular weight of less than 2,000 Dalton (da).

171. The method of any one of Embodiments 149-170, wherein the compound is a natural product.

172. A system including one or more computing devices, comprising:
one or more non-transitory computer-readable storage media including instructions; and
one or more processors coupled to the one or more storage media, the one or more processors configured to execute the instructions to:
   receive mass spectrometry (MS) data, wherein the MS data comprises a plurality of mass-to-charge values and a precursor mass associated with a compound;
   generate a plurality of tokens based at least in part on the plurality of mass-to-charge values and precursor mass;
   input the plurality of tokens into a bidirectional transformer-based machine-learning model trained to generate one or more predictions of a chemical structure of the compound based on the plurality of tokens; and
   output, by the bidirectional transformer-based machine-learning model, the one or more predictions of the chemical structure of the compound.

173. A non-transitory computer-readable medium comprising instructions that, when executed by one or more processors of one or more computing devices, cause the one or more processors to:
receive mass spectrometry (MS) data, wherein the MS data comprises a plurality of mass-to-charge values and a precursor mass value associated with a compound;
generate a plurality of tokens based at least in part on the plurality of mass-to-charge values and precursor mass;
input the plurality of tokens into a bidirectional transformer-based machine-learning model trained to generate one or more predictions of a chemical structure of the compound based on the plurality of tokens; and
output, by the bidirectional transformer-based machine-learning model, the one or more predictions of the chemical structure of the compound.

174. A method for training a transformer-based machine-learning model to identify a chemical structure of a compound based on a mass spectrometry (MS) data, the method comprising, by one or more computing devices:
accessing a data set of mass spectra data, wherein the data set of mass spectra data comprises a plurality of mass-to-charge values and a precursor mass associated with a compound;
generating a plurality of tokens based on the plurality of mass-to-charge values and the precursor mass, wherein the plurality of tokens comprises a set of one or more corrupted tokens and uncorrupted tokens, and wherein the one or more corrupted tokens are predetermined to selectively correspond to the precursor mass; and
inputting the plurality of tokens into the transformer-based machine-learning model to generate a prediction of the one or more corrupted tokens based on the uncorrupted tokens, the prediction of the one or more corrupted tokens corresponding to an original sequence of tokens representative of the plurality of mass-to-charge values and the precursor mass.

175. The method of Embodiment 174, wherein the transformer-based machine-learning model is further trained by:
computing a cross-entropy loss value based on a comparison of the prediction of the one or more corrupted tokens and the original sequence of tokens representative of the plurality of mass-to-charge values and the precursor mass; and
updating the transformer-based machine-learning model based on the cross-entropy loss value.

176. The method of Embodiment 175, wherein training the transformer-based machine-learning model comprises pre-training the transformer-based machine-learning model, the method further comprising:
fine-tuning the pre-trained transformer-based machine-learning model.

177. The method of any one of Embodiments 175 or 176, wherein the cross-entropy loss value comprises a weighted cross-entropy loss value.

178. The method of Embodiment any one of Embodiments 175-177, wherein the weighted cross-entropy loss value is expressed as:

$$ℒ = {\sum }_{S ∈ Structures} \frac{1}{\vert MS ( S ) + κ \vert} {\sum }_{t ∈ MS ( S )} log ( p ( S \vert t ) ) ) .$$

179. The method of Embodiment 176, wherein fine-tuning the pre-trained transformer-based machine-learning model comprises:
accessing a second data set of mass spectra data, wherein the second data second set of mass spectra data comprises a second plurality of mass-to-charge values and a second precursor mass associated with a compound;
generating a second plurality of tokens based on the second plurality of mass-to-charge values and the second precursor mass; and
inputting the second plurality of tokens into the pre-trained transformer-based machine-learning model to generate a prediction of one or more chemical structures of the compound based on the second plurality of tokens.

180. The method of Embodiment 179, wherein the fine-tuned transformer-based machine-learning model is further trained by:
computing a second cross-entropy loss value based on a comparison of the prediction of the one or more chemical structures and a second original sequence of tokens corresponding to the second plurality of mass-to-charge values and the second precursor mass; and
updating the fine-tuned transformer-based machine-learning model based on the second cross-entropy loss value.

181. The method of Embodiment 179 or 180, wherein the prediction of the one or more chemical structures comprises one or more deep simplified molecular-input line-entry system (DeepSMILES) strings.

182. The method of any one of Embodiments 179-181, wherein the prediction of the one or more chemical structures comprises one or more simplified molecular-input line-entry system (SMILES) strings.

183. The method of Embodiment any one of Embodiments 179-182, wherein the prediction of the one or more chemical structures comprises one or more self-referencing embedded strings (SELFIES).

184. The method of Embodiment 174-183, wherein the one or more corrupted tokens are predetermined to selectively correspond to the precursor mass in 50% of training iterations of the transformer-based machine-learning model.

185. The method of Embodiment any one of Embodiments 174-184, wherein the one or more corrupted tokens are predetermined to selectively correspond to the precursor mass in a heuristically-determined number of training iterations of the transformer-based machine-learning model.

186. The method of Embodiment any one of Embodiments 174-185, wherein the MS data comprises a plurality of mass-to-charge values and the precursor mass obtained from tandem mass spectrometry (MS2) performed on the compound.

187. The method of Embodiment any one of Embodiments 174-186, wherein the MS data comprises a plurality of mass-to-charge values and the precursor mass obtained from ion mobility mass spectrometry (IM-MS) performed on the compound.

188. The method of Embodiment any one of Embodiments 174-187, wherein the plurality of tokens comprises one or more masked tokens and unmasked tokens, the method further comprising:
inputting the second plurality of tokens into the transformer-based machine-learning model to generate a prediction of the one or more masked tokens based on the unmasked tokens, the prediction of the one or more masked tokens corresponding to the prediction of the plurality of candidates of the chemical structure of the compound.

189. The method of any one of Embodiments 174-188, further comprising performing a process to corrupt the one or more corrupted tokens included in the set of one or more corrupted tokens and uncorrupted tokens.

190. The method of Embodiment 188, wherein the process to corrupt the one or more corrupted tokens comprises a process to corrupt the precursor mass.

191. The method of any one of Embodiments 188-190, wherein the process to corrupt the one or more corrupted tokens comprises a token deletion process, a token masking process, a text infilling process, a text string permutation process, or a sequence rotation process.

192. The method of any one of Embodiments 174-191, wherein the transformer-based machine-learning model comprises a bidirectional and auto-regressive transformer (BART) model.

193. The method of any one of Embodiments 174-192, wherein the transformer-based machine-learning model comprises a bidirectional encoder representations for transformer (BERT) model.

194. The method of any one of Embodiments 174-193, wherein the transformer-based machine-learning model comprises a generative pre-trained transformer (GPT) model.

195. The method of any one of Embodiments 174-194, wherein the transformer-based machine-learning model is further trained by:
accessing a dataset of small molecule data, wherein the dataset of small molecule data is not associated with MS data;
generating a set of text strings representative of the dataset of small molecule data; and
inputting the set of text strings into the transformer-based machine-learning model to generate a prediction of one or more chemical structures corresponding to the dataset of small molecule data.

196. The method of Embodiment 195, wherein the small molecule data comprises a molecule having a mass of 900 Dalton (da) or less.

197. The method of Embodiment 195 or Embodiment 196, wherein the small molecule data comprises a molecule having a mass of 600 Dalton (da) or less.

198. The method of any one of Embodiments 195-197, wherein the small molecule data comprises a molecule having a mass of 500 Dalton (da) or less.

199. The method of Embodiment any one of Embodiments 195-198, wherein the small molecule data comprises a molecule having a mass of 300 Dalton (da) or less.

200. A system including one or more computing devices, comprising:
one or more non-transitory computer-readable storage media including instructions; and
one or more processors coupled to the one or more storage media, the one or more processors configured to execute the instructions to:
   access a data set of mass spectra data, wherein the data set of mass spectra data comprises a plurality of mass-to-charge values and a precursor mass corresponding to a compound;
   generate a plurality of tokens based on the plurality of mass-to-charge values and the precursor mass, wherein the plurality of tokens comprises a set of one or more corrupted tokens and uncorrupted tokens, and wherein the one or more corrupted tokens are predetermined to selectively correspond to the precursor mass; and
   input the plurality of tokens into the transformer-based machine-learning model to generate a prediction of the one or more corrupted tokens based on the uncorrupted tokens, the prediction of the one or more corrupted tokens corresponding to an original sequence of tokens representative of the plurality of mass-to-charge values and the precursor mass.

201. A non-transitory computer-readable medium comprising instructions that, when executed by one or more processors of one or more computing devices, cause the one or more processors to:
access a data set of mass spectra data, wherein the data set of mass spectra data comprises a plurality of mass-to-charge values and a precursor mass corresponding to a compound;
generate a plurality of tokens based on the plurality of mass-to-charge values and the precursor mass, wherein the plurality of tokens comprises a set of one or more corrupted tokens and uncorrupted tokens, and wherein the one or more corrupted tokens are predetermined to selectively correspond to the precursor mass; and
input the plurality of tokens into the transformer-based machine-learning model to generate a prediction of the one or more corrupted tokens based on the uncorrupted tokens, the prediction of the one or more corrupted tokens corresponding to an original sequence of tokens representative of the plurality of mass-to-charge values and the precursor mass.

202. A method for training a transformer-based machine-learning model to identify a chemical property of a compound based on a mass spectrometry (MS) data, the method comprising, by one or more computing devices:
receiving mass spectrometry (MS) data, wherein the MS data comprises a plurality of mass-to-charge values and a precursor mass associated with a compound;
generating a plurality of tokens based on the plurality of mass-to-charge values and the precursor mass, wherein the plurality of tokens comprises a set of one or more masked tokens and unmasked tokens, and wherein the one or more masked tokens are predetermined to selectively correspond to the precursor mass;
inputting the plurality of tokens into a transformer-based machine-learning model to generate a prediction of the one or more masked tokens based on the unmasked tokens; and
generating, by the transformer-based machine-learning model, the prediction of the one or more masked tokens, the prediction of the one or more masked tokens corresponding at least in part to a prediction of one or more chemical properties of the compound.

203. The method of Embodiment 202, wherein the transformer-based machine-learning model comprises a bidirectional encoder representations for transformer (BERT) model.

204. The method of Embodiment 202 or 203, wherein the MS data comprises a plurality of mass-to-charge values and precursor mass obtained from tandem mass spectrometry (MS2) performed on the compound.

205. The method of any one of Embodiments 202-204, wherein the MS data comprises a plurality of mass-to-charge values and precursor mass obtained from ion mobility mass spectrometry (IM-MS) performed on the compound.

206. The method of Embodiment any one of Embodiments 202-205, wherein training the transformer-based machine-learning model comprises pre-training the transformer-based machine-learning model, the method further comprising:
fine-tuning the pre-trained transformer-based machine-learning model to identify the chemical property of the compound.

207. The method of any one of Embodiments 202-206, wherein the transformer-based machine-learning model is further trained by:
computing a loss value based on a comparison of the prediction of the one or more masked tokens and an input sequence of tokens corresponding to the plurality of mass-to-charge values and the precursor mass; and
updating the transformer-based machine-learning model based on the computed loss value.

208. The method of Embodiment 207, wherein the loss value comprises a weighted cross-entropy loss value.

209. The method of Embodiment 208, wherein the loss value is expressed as:

$$ℒ = {\sum }_{S ∈ Structures} \frac{1}{\vert MS ( S ) + κ \vert} {\sum }_{t ∈ MS ( S )} log ( p ( S \vert t ) ) ) .$$

210. The method of any one of Embodiments 202-209, wherein the transformer-based machine-learning model is associated with a predetermined vocabulary, and wherein the predetermined vocabulary comprises one or more sets of tokens corresponding to a curated dataset of experimental simplified molecular-input line-entry system (SMILES) strings.

211. The method of any one of Embodiments 202-210, wherein the set of one or more masked tokens comprises at least 15% of a total number of the plurality of tokens.

212. The method of any one of Embodiments 202-211, the prediction of the one or more chemical properties comprises a prediction of a natural product class of the compound.

213. The method of any one of Embodiments 202-212, the prediction of the one or more chemical properties comprises a prediction of a LogP value associated with the compound.

214. The method of any one of Embodiments 202-213, the prediction of the one or more chemical properties comprises a prediction of a number of hemoglobin receptors of the compound.

215. The method of any one of Embodiments 202-214, the prediction of the one or more chemical properties comprises a prediction of a number of hemoglobin donors of the compound.

216. The method of Embodiment any one of Embodiments 202-215, the prediction of the one or more chemical properties comprises a prediction of a polar surface area of the compound.

217. The method of any one of Embodiments 202-216, the prediction of the one or more chemical properties comprises a prediction of a number of rotatable bonds of the compound.

218. The method of any one of Embodiments 202-217, the prediction of the one or more chemical properties comprises a prediction of a number of aromatic rings of the compound.

219. The method of any one of Embodiments 202-218, the prediction of the one or more chemical properties comprises a prediction of a number of aliphatic rings of the compound.

220. The method of any one of Embodiments 202-219, the prediction of the one or more chemical properties comprises a prediction of a number of heteroatoms of the compound.

221. The method of any one of Embodiments 202-220, the prediction of the one or more chemical properties comprises a prediction of a fraction of sp³ carbon atoms (Fsp³) of the compound.

222. The method of any one of Embodiments 202-221, the prediction of the one or more chemical properties comprises a prediction of a molecular weight of the compound.

223. The method of any one of Embodiments 202-222, the prediction of the one or more chemical properties comprises a prediction of an adduct or fragment associated with the compound.

224. The method of any one of Embodiments 202-223, wherein the one or more masked tokens are predetermined to selectively correspond to the precursor mass in 50% of training iterations of the transformer-based machine-learning model.

225. The method of any one of Embodiments 202-224, wherein the one or more masked tokens are predetermined to selectively correspond to the precursor mass in a heuristically-determined number of training iterations of the transformer-based machine-learning model.

226. A system including one or more computing devices, comprising:
one or more non-transitory computer-readable storage media including instructions; and
one or more processors coupled to the one or more storage media, the one or more processors configured to execute the instructions to:
   receive mass spectrometry (MS) data, wherein the MS data comprises a plurality of mass-to-charge values and a precursor mass associated with a compound;
   generate a plurality of tokens based on the plurality of mass-to-charge values and the precursor mass, wherein the plurality of tokens comprises a set of one or more masked tokens and unmasked tokens, and wherein the one or more masked tokens are predetermined to selectively correspond to the precursor mass;
   input the plurality of tokens into a transformer-based machine-learning model to generate a prediction of the one or more masked tokens based on the unmasked tokens; and
   generate, by the transformer-based machine-learning model, the prediction of the one or more masked tokens, the prediction of the one or more masked tokens corresponding at least in part to a prediction of one or more chemical properties of the compound.

227. A non-transitory computer-readable medium comprising instructions that, when executed by one or more processors of one or more computing devices, cause the one or more processors to:
receive mass spectrometry (MS) data, wherein the MS data comprises a plurality of mass-to-charge values and a precursor mass associated with a compound;
generate a plurality of tokens based on the plurality of mass-to-charge values and the precursor mass, wherein the plurality of tokens comprises a set of one or more masked tokens and unmasked tokens, and wherein the one or more masked tokens are predetermined to selectively correspond to the precursor mass;
input the plurality of tokens into a transformer-based machine-learning model to generate a prediction of the one or more masked tokens based on the unmasked tokens; and
generate, by the transformer-based machine-learning model, the prediction of the one or more masked tokens, the prediction of the one or more masked tokens corresponding at least in part to a prediction of one or more chemical properties of the compound.

The embodiments disclosed herein are only examples, and the scope of this disclosure is not limited to them. Embodiments according to this disclosure are in particular disclosed in the attached claims directed to a method, a storage medium, a system and a computer program product, wherein any feature mentioned in one claim category, e.g. method, can be claimed in another claim category, e.g. system, as well. The dependencies or references back in the attached claims are chosen for formal reasons only. However, any subject matter resulting from a deliberate reference back to any previous claims (in particular multiple dependencies) can be claimed as well, so that any combination of claims and the features thereof are disclosed and can be claimed regardless of the dependencies chosen in the attached claims. The subject-matter which can be claimed comprises not only the combinations of features as set out in the attached claims but also any other combination of features in the claims, wherein each feature mentioned in the claims can be combined with any other feature or combination of other features in the claims. Furthermore, any of the embodiments and features described or depicted herein can be claimed in a separate claim and/or in any combination with any embodiment or feature described or depicted herein or with any of the features of the attached claims.

The scope of this disclosure encompasses all changes, substitutions, variations, alterations, and modifications to the example embodiments described or illustrated herein that a person having ordinary skill in the art would comprehend. The scope of this disclosure is not limited to the example embodiments described or illustrated herein. Moreover, although this disclosure describes and illustrates respective embodiments herein as including particular components, elements, feature, functions, operations, or steps, any of these embodiments may include any combination or permutation of any of the components, elements, features, functions, operations, or steps described or illustrated anywhere herein that a person having ordinary skill in the art would comprehend. Furthermore, reference in the appended claims to an apparatus or system or a component of an apparatus or system being adapted to, arranged to, capable of, configured to, enabled to, operable to, or operative to perform a particular function encompasses that apparatus, system, component, whether or not it or that particular function is activated, turned on, or unlocked, as long as that apparatus, system, or component is so adapted, arranged, capable, configured, enabled, operable, or operative. Additionally, although this disclosure describes or illustrates certain embodiments as providing particular advantages, certain embodiments may provide none, some, or all of these advantages.

1-30. (canceled)

31. A method for identifying a chemical structure of a compound based on mass spectrometry (MS) data, the method comprising, by one or more computing devices:
receiving mass spectrometry (MS) data, wherein the MS data comprises a plurality of mass-to-charge values associated with fragments obtained from mass spectrometry performed on the compound;
generating a plurality of tokens based on the plurality of mass-to-charge values;
inputting the plurality of tokens into a bidirectional transformer-based machine-learning model trained to generate one or more predictions of a chemical structure of the compound based on the plurality of tokens; and
outputting, by the bidirectional transformer-based machine-learning model, the one or more predictions of the chemical structure of the compound.

32. The method of claim 31, wherein the one or more predictions of the chemical structure of the compound comprises a plurality of candidates of the chemical structure of the compound.

33. The method of claim 31, wherein the bidirectional transformer-based machine-learning model comprises a bidirectional and auto-regressive transformer (BART) model.

34. The method of claim 31, wherein the bidirectional transformer-based machine-learning model comprises a bidirectional encoder representations for transformer (BERT) model.

35. The method of claim 31, wherein the bidirectional transformer-based machine-learning model comprises a generative pre-trained transformer (GPT) model.

36. The method of claim 31, further comprising generating an image of the plurality of candidates of the chemical structure of the compound.

37. The method of claim 31, wherein the mass spectrometry comprises a tandem mass spectrometry technique.

38. The method of claim 31, wherein the mass spectrometry is an electrospray ionization mass spectrometry technique.

39. The method of claim 38, wherein the electrospray ionization mass spectrometry technique comprises a positive-ion mode mass spectrometry technique.

40. The method of claim 39, wherein the electrospray ionization mass spectrometry technique comprises a negative-ion mode mass spectrometry technique.

41. The method of claim 31, wherein the mass spectrometry comprises use of a data-dependent acquisition technique.

42. The method of claim 31, wherein the mass spectrometry technique comprises use of a data-independent acquisition technique.

43. The method of claim 31, wherein the mass spectrometry comprises use of a mass spectrometer.

44. The method of claim 43, wherein the mass spectrometer has a mass accuracy of 25 ppm or greater.

45. The method of claim 31, wherein the mass spectrometry comprises an upstream separation technique.

46. The method of claim 45, wherein the separation technique is a liquid chromatography technique.

47. The method of claim 46, wherein the liquid chromatography technique is an online liquid chromatography technique.

48. The method of claim 31, further comprising subjecting a sample comprising the compound to mass spectrometry to generate the MS data.

49. The method of claim 48, further comprising obtaining the sample.

50. The method of claim 48, wherein the sample is a natural sample or a derivative thereof.

51. The method of claim 31, wherein the sample comprises a plant extract or a derivative thereof.

52. The method of claim 31, wherein the compound is a small molecule having a molecular weight of less than 2,000 Dalton (da).

53. The method of claim 31, wherein the compound is a natural product.

54. A system including one or more computing devices, comprising:
one or more non-transitory computer-readable storage media including instructions; and
one or more processors coupled to the one or more storage media, the one or more processors configured to execute the instructions to:
receive mass spectrometry (MS) data, wherein the MS data comprises a plurality of mass-to-charge values associated with fragments obtained from mass spectrometry performed on the compound;
generate a plurality of tokens based on the plurality of mass-to-charge values;
input the plurality of tokens into a bidirectional transformer-based machine-learning model trained to generate one or more predictions of a chemical structure of the compound based on the plurality of tokens; and
output, by the bidirectional transformer-based machine-learning model, the one or more predictions of the chemical structure of the compound.

55. A non-transitory computer-readable medium comprising instructions that, when executed by one or more processors of one or more computing devices, cause the one or more processors to:
receive mass spectrometry (MS) data, wherein the MS data comprises a plurality of mass-to-charge values associated with fragments obtained from mass spectrometry performed on the compound;
generate a plurality of tokens based on the plurality of mass-to-charge values;
input the plurality of tokens into a bidirectional transformer-based machine-learning model trained to generate one or more predictions of a chemical structure of the compound based on the plurality of tokens; and
output, by the bidirectional transformer-based machine-learning model, the one or more predictions of the chemical structure of the compound.

56-90. (canceled)

91. A method for training a transformer-based machine-learning model to identify a chemical property of a compound based on a mass spectrometry (MS) data, the method comprising, by one or more computing devices:
receiving mass spectrometry (MS) data, wherein the MS data comprises a plurality of mass-to-charge values obtained from mass spectrometry performed on a compound;
generating a plurality of tokens based on the plurality of mass-to-charge values, wherein the plurality of tokens comprises a set of one or more masked tokens and unmasked tokens;
inputting the plurality of tokens into a transformer-based machine-learning model to generate a prediction of the one or more masked tokens based on the unmasked tokens; and
generating, by the transformer-based machine-learning model, the prediction of the one or more masked tokens, the prediction of the one or more masked tokens corresponding at least in part to a prediction of one or more chemical properties of the compound.

92-132. (canceled)

133. A method for training a transformer-based machine-learning model to identify a chemical structure of a compound based on a mass spectrometry (MS) data, the method comprising, by one or more computing devices:
accessing a data set of one or more simplified molecular-input line-entry system (SMILES) strings corresponding to a compound;
generating a plurality of tokens based on the one or more SMILES strings, wherein the plurality of tokens comprises a set of one or more corrupted tokens and uncorrupted tokens; and
inputting the plurality of tokens into the transformer-based machine-learning model to generate a prediction of the one or more corrupted tokens based on the uncorrupted tokens, the prediction of the one or more corrupted tokens corresponding to an original sequence of tokens representative of the one or more SMILES strings.

134-227. (canceled)
